(19) United States
(12) Patent Application Publication
(10) Pub. No.: US 2025/0339555 A1
(43) Pub. Date: Nov. 6, 2025

(54) FORMULATIONS FOR SUPRACHOROIDAL ADMINISTRATION SUCH AS FORMULATIONS WITH AGGREGATE FORMATION

(71) Applicant: REGENXBIO INC., Rockville, MD (US)

(72) Inventors: Jared BEE, Gaithersburg, MD (US); Tristan James MARSHALL, Potomac, MD (US); Sherri VAN EVEREN, Menlo Park, CA (US); Stephen Joseph PAKOLA, Irvington, NY (US); Ewa BUDZYNSKI, Deforest, WI (US); Nicholas Alexander Piers Sasha BUSS, Rockville, MD (US)

(21) Appl. No.: 18/854,315

(22) PCT Filed: Apr. 5, 2023
(86) PCT No.: PCT/US2023/065365

§ 371 Date: Oct. 4, 2024

Related U.S. Application Data
(60) Provisional application No. 63/328,252, filed on Apr. 6, 2022.

(51) Int. Cl. A61K 48/00; A61K 9/00; A61K 47/02; A61K 47/26; A61K 47/34; A61P 27/02; C12N 15/86

(52) U.S. Cl. CPC A61K 48/0041; A61K 9/0048; A61K 47/02; A61K 47/26; A61K 47/34; A61K 48/0075; A61P 27/02; C12N 15/86; C12N 2750/14143

(57) ABSTRACT

Provided herein are pharmaceutical compositions for administration to a suprachoroidal space of an eye of a subject. The pharmaceutical compositions can include a recombinant adeno-associated virus (AAV) encoding a transgene. Also provided herein are methods for treating or preventing a disease in a subject by administering a therapeutically effective amount of the pharmaceutical compositions to the subject in need.

### PRIORITY

This application claims the benefit of priority to U.S. Ser. No. 63/328,252 filed Apr. 6, 2022, which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “12656-163-228_SEQLISTING.xml”, was created on Mar. 30, 2023 and is 49,621 bytes in size.

### 1. BACKGROUND OF THE INVENTION

The human eye is a highly intricate and highly developed sensory organ, which is prone to a host of diseases and disorders. About 285 million people in the world are visually impaired, of whom 39 million are blind and 246 million have moderate to severe visual impairment (World Health Organization, 2012, “Global Data On Visual Impairments 2010,” Geneva: World Health Organization). Some of the leading causes of blindness are cataract (47%), glaucoma (12%), age-related macular degeneration (AMD) (9%), and diabetic retinopathy (5%) (World Health Organization, 2007, “Global Initiative For The Elimination Of Avoidable Blindness: Action Plan 2006-2011,” Geneva: World Health Organization).

Gene therapy has been employed in treating certain eye diseases (see, e.g. International Patent Application No. PCT/US2017/027650 (International Publication No. WO 2017/181021 A1)). Adeno-associated viruses (AAV) are an attractive tool for gene therapy due to properties of non-pathogenicity, broad host and cell type tropism range of infectivity, including both dividing and non-dividing cells, and ability to establish long-term transgene expression (e.g., Gonçalves, 2005, Virology Journal, 2:43).

Current methods used for ocular gene therapy (e.g., by intravitreous or subretinal administrations) are invasive and have serious setbacks, such as, increased risk of cataract, retinal detachment, and separation of photoreceptors from the retinal pigment epithelium (RPE) in the fovea. There is a significant unmet medical need for therapies that improve or eliminate the setbacks from current ocular gene therapy.

Adeno-associated virus (AAV), a member of the Parvoviridae family designated Dependovirus, is a small nonenveloped, icosahedral virus with single-stranded linear DNA genomes of approximately 4.7-kilobases (kb) to 6 kb. The properties of non-pathogenicity, broad host and cell type tropism range of infectivity, including both dividing and non-dividing cells, and ability to establish long-term transgene expression make AAV an attractive tool for gene therapy (e.g., Gonçalves, 2005, Virology Journal, 2.43).

The suprachoroidal space (SCS) is a region between the sclera and the choroid that expands upon injection of the drug solution (Habot-Wilner, 2019). The SCS space recovers to its pre-injection size as the injected solution is cleared by physiologic processes. The drug solution diffuses within SCS and is absorbed into adjacent tissues. Capillaries in the choroid are permeable to low molecular weight osmolytes. The present disclosure addresses an unmet need of providing pharmaceutical compositions that lead to longer residence time in the suprachoroidal space, and consequently improved efficacy.

### 2. SUMMARY OF THE INVENTION

In one aspect, provided herein is a pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical composition has an amount of viral vector aggregation such that when administered to an eye of a pig: a. the clearance time of the pharmaceutical composition is between about 5 days and about 15 days; and b. the thickness of the SCS at the site of injection is between about 400 μm and about 800 μm at a time within one hour of administration; and c. the circumferential spread of the pharmaceutical composition from the site of injection is about one-eighth or less of a surface of the choroid at a time within about one hour of administration.

In some embodiments, the pharmaceutical composition comprises a recombinant adeno-associated virus (AAV) vector comprising an expression cassette encoding a transgene, and wherein the pharmaceutical composition comprises an ionic strength of at most about 200 mM prior to suprachoroidal administration.

In some embodiments, the pharmaceutical composition comprises a recombinant adeno-associated virus (AAV) vector comprising an expression cassette encoding a transgene, and wherein the pharmaceutical composition comprises at least about 3% aggregated recombinant AAV prior to suprachoroidal administration.

In some embodiments, the clearance time after suprachoroidal administration of the pharmaceutical composition is equal to or greater than the clearance time after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition. In some embodiments, a circumferential spread after suprachoroidal administration of the pharmaceutical composition is smaller as compared to a circumferential spread after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition. In some embodiments, a thickness at a site of injection after suprachoroidal administration of the pharmaceutical composition is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition. In some embodiments, an expression level of the transgene is detected in the eye for a longer period of time after suprachoroidal administration of the pharmaceutical composition as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition. In some embodiments, the concentration of the transgene product in the eye after suprachoroidal administration of the pharmaceutical composition is equal to or higher as compared to the concentration of the transgene product in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition. In some embodiments, the concentration of the transgene product in the back of the eye (e.g., retina) after suprachoroidal administration of the pharmaceutical composition is equal to or higher as compared to the concentration of the transgene product in the back of the eye after suprachoroidal administration of a reference pharmaceutical composition, and/or the concentration of the transgene product in the outer layer of the eye (e.g., sclera) after suprachoroidal administration of the pharmaceutical composition is lower than the concentration of the transgene product in the outer layer of the eye after suprachoroidal administration of a reference pharmaceutical composition disclosed herein.

In some embodiments, the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

In some embodiments, the clearance time is from the SCS or from the eye. In some embodiments, the clearance time is the time required for the pharmaceutical composition and/or the recombinant adeno-associated virus (AAV) vector to not be detectable in the SCS by any standard method. In some embodiments, the clearance time when the pharmaceutical composition and/or the recombinant adeno-associated virus (AAV) vector is present in the SCS in an amount that is at most about 2% or at most about 5% of the amount detectable by any standard method. In some embodiments, the clearance time is the amount of time required following injection for the thickness at the site of injection to decrease to about 1 nm or less, about 2 nm or less, about 5 nm or less, about 10 nm or less, about 25 nm or less, about 50 nm or less, about 100 nm or less, about 200 nm or less, or about 500 nm or less. In some embodiments, the clearance time is the amount of time required following injection for the thickness at the site of injection to decrease to about 500 nm or less, about 200 nm or less, about 100 nm or less, about 50 nm or less, about 25 nm or less, about 10 nm or less, or is undetectable. In some embodiments, the clearance time is the amount of time required following injection for the pharmaceutical composition to spread circumferentially from the site of injection to cover about one-sixteenth or more, about one-eighth or more, about one-fourth or more, about one-half or more, about three-fourths or more, or all of the circumference of the choroid of the eye.

In some embodiments, the transgene is not an anti-human vascular endothelial growth factor (anti-VEGF) antibody. In some embodiments, the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the recombinant AAV is AAV8. In some embodiments, wherein the recombinant AAV is AAV9. In some embodiments, the pharmaceutical composition has an ionic strength of about or at most about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM. In some embodiments, the pharmaceutical composition comprises at least about or about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% aggregated recombinant AAV.

In some embodiments, the pharmaceutical composition has an ionic strength of about or of at most about 40 mM. In some embodiments, the pharmaceutical composition has an ionic strength of about or of at most about 135 mM. In some embodiments, the pharmaceutical composition has an ionic strength of about or of at most about 20 mM. In some embodiments, the pharmaceutical composition has an average recombinant AAV diameter of about or at least about: 28 nm, 29 nm, 30 nm, 31 nm, 32 nm, 33 nm, 34 nm, 35 nm, 36 nm, 37 nm, 38 nm, 39 nm, 40 nm, 45 nm, 50 nm, 55 nm, 60 nm, 65 nm, 70 nm, 75 nm, 80 nm, 85 nm, 90 nm, 95 nm, or about or at least about 100 nm (e.g., as measured with dynamic light scattering).

In some embodiments, the pharmaceutical composition has an average recombinant AAV diameter that is at least 2 times higher, at least 3 times higher, at least 4 times higher, at least 5 times higher, at least 6 times higher, at least 7 times higher, at least 8 times higher, at least 9 times higher, at least 10 times higher, at least 15 times higher, at least 20 times higher, at least 50 times higher, at least 100 times higher, at least 5% higher, at least 10% higher, at least 15% higher, at least 20% higher, at least 25% higher, at least 30% higher, at least 35% higher, at least 40%, at least 45% higher, at least 50% higher, at least 55% higher, at least 60% higher, at least 65% higher, at least 70% higher, at least 75% higher, at least 80% higher, at least 85% higher, at least 90% higher, at least 95% higher, at least 100% higher, at least 150% higher, or at least 200% higher, at least 250% higher, or at least 300%, at least 400% higher, or at least 500% higher than an average recombinant AAV diameter in the reference pharmaceutical composition. In some embodiments, the circumferential spread after suprachoroidal administration of the pharmaceutical composition is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%. In some embodiments, the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%.

In some embodiments, the clearance time after suprachoroidal administration of the pharmaceutical composition is of about 6 days to about 15 days, about 7 days to about 15 days, about 8 days to about 15 days, about 9 days to about 15 days, about 10 days to about 15 days, about 11 days to about 15 days, about 12 days to about 15 days, about 13 days to about 15 days, about 14 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, or about 5 days to about 6 days. In some embodiments, the clearance time after suprachoroidal administration of the pharmaceutical composition is not prior to about 5 days, 6 days, 7 days, 8 days, 9 days, 10 days, 11 days, 12 days, 13 days, 14 days, or 15 days. In some embodiments, the clearance time after suprachoroidal administration of the reference pharmaceutical composition is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.

In some embodiments, the clearance time after suprachoroidal administration of the pharmaceutical composition is of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days.

In some embodiments, the clearance time after suprachoroidal administration of the pharmaceutical composition is not prior to about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the clearance time after suprachoroidal administration of the reference pharmaceutical composition is of at most about 1 day, 2 days, 3 days, 4 days, 5 days, 6 days, 7 days, 8 days, 9 days, 10 days, 11 days, 12 days, 13 days, or 14 days. In some embodiments, the clearance time is from the SCS or from the eye. In some embodiments, the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm. In some embodiments, the thickness at the site of injection after suprachoroidal administration of the pharmaceutical composition is of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm. In some embodiments, the thickness at the site of injection after suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 μm.

In some embodiments, the thickness of the SCS at the site of injection is about 400 μm to about 700 μm, about 400 μm to about 600 μm, about 400 μm to about 500 μm, about 500 μm to about 800 μm, about 600 μm to about 800 μm, 700 μm to about 800 μm at a time within one hour of administration. In some embodiments, the time within one hour of administration is within about 5 minutes of administration, within about 10 minutes of administration, within about 15 minutes of administration, within about 20 minutes of administration, within about 30 minutes of administration, within about 45 minutes of administration, or within about 60 minutes of administration.

In some embodiments, the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

In some embodiments, the circumferential spread of the pharmaceutical composition from the site of injection is about one-eighth or less of a surface of the choroid at a time within about 5 minutes of administration, within about 10 minutes of administration, within about 15 minutes of administration, within about 20 minutes of administration, within about 30 minutes of administration, within about 45 minutes of administration, or within about 60 minutes of administration. In some embodiments, the circumferential spread from the site of injection is about one-sixteenth or less of a surface of the choroid.

In some embodiments, the concentration of the transgene product in the eye after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the longer period of time after suprachoroidal administration of the pharmaceutical composition is longer by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the transgene is detected in the eye after suprachoroidal administration of the pharmaceutical composition for at least about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days. In some embodiments, the transgene is detected in the eye after suprachoroidal administration of the reference pharmaceutical composition for at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

In some embodiments, the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the recombinant AAV stability in the pharmaceutical composition is at least about 50% of the recombinant AAV stability in the reference pharmaceutical composition. In some embodiments, the recombinant AAV stability is determined by infectivity of the recombinant AAV. In some embodiments, the recombinant AAV stability is determined by a level of free DNA released by the recombinant AAV. In some embodiments, the pharmaceutical composition comprises at least about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less free DNA as compared to a level of free DNA in the reference pharmaceutical composition. In some embodiments, the recombinant AAV in the pharmaceutical composition has an infectivity that is about 50% lower, about the same, or at least about 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition.

In some embodiments, the transgene is a transgene suitable to treat, or otherwise ameliorate, prevent or slow the progression of a disease of interest. In some embodiments, the human subject is diagnosed with nAMD (wet AMD), dry AMD, retinal vein occlusion (RVO), diabetic macular edema (DME), or diabetic retinopathy (DR), or Batten disease. In other embodiments, the human subject is diagnosed with glaucoma or non-infectious uveitis. In some embodiments, the human subject is diagnosed with kallikrein-related disease.

In some embodiments, the AAV encodes Palmitoyl-Protein Thioesterase 1 (PPT1) or Tripeptidyl-Peptidase 1 (TPP1). In other embodiments, the AAV encodes anti-kallikrein antibody or antigen-binding fragment, anti-TNF fusion protein, anti-TNF antibody or antigen-binding fragment, anti-C3 antibody or antigen-binding fragment, or anti-C5 antibody or antigen-binding fragment.

In some embodiments, the amount of the recombinant AAV genome copies is based on a vector genome concentration. In some embodiments, the amount of the recombinant AAV genome copies is based on genome copies per administration. In some embodiments, the amount of the recombinant AAV genome copies is based on total genome copies administered to the human subject. In some embodiments, the genome copies per administration is the genome copies of the recombinant AAV per suprachoroidal administration. In some embodiments, the total genome copies administered is the total genome copies of the recombinant AAV administered suprachoroidally.

In some embodiments, the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 1.5×10¹³ GC/mL, about 2×10¹³ GC/mL, or about 3×10¹³ GC/mL. In some embodiments, the total genome copies administered is about 6.0×10¹⁰ genome copies, about 1.6×10¹¹ genome copies, about 2.5×10¹¹ genome copies, about 5.0×10¹¹ genome copies, about 1.5×10¹² genome copies, about 3×10¹² genome copies, about 1.0×10¹² genome copies, about 2.5×10¹² genome copies, or about 3.0×10¹³ genome copies. In some embodiments, the genome copies per administration is about 6.0×10¹⁰ genome copies, about 1.6×10¹¹ genome copies, about 2.5×10¹¹ genome copies, about 5.0×10¹¹ genome copies, about 3×10¹² genome copies, about 1.0×10¹² genome copies, about 1.5×10¹² genome copies, about 2.5×10¹² genome copies, or about 3.0×10¹³ genome copies.

In some embodiments, the pharmaceutical composition is administered once, twice, three times, four times, five times, six times, seven times, eight times, nine times, ten times, fifteen times, twenty times, twenty-five times, or thirty times. In some embodiments, the reference pharmaceutical composition is administered once, twice, three times, four times, five times, six times, seven times, eight times, nine times, ten times, fifteen times, twenty times, twenty-five times, or thirty times. In some embodiments, the pharmaceutical composition is administered once in one day, twice in one day, three times in one day, four times in one day, five times in one day, six times in one day, or seven times in one day. In some embodiments, the reference pharmaceutical composition is administered once in one day, twice in one day, three times in one day, four times in one day, five times in one day, six times in one day, or seven times in one day. In some embodiments, the reference pharmaceutical composition comprises DPBS and sucrose.

In one aspect, provided herein is a method of preparing a pharmaceutical composition comprising: (i) preparing a composition comprising phosphate-buffered saline, sucrose, and a recombinant adeno-associated virus (AAV) vector comprising an expression cassette encoding a transgene; and (ii) admixing a solution comprising phosphate-buffered saline and sucrose to the composition, wherein the pharmaceutical composition has lower ionic strength and/or a higher level of aggregated recombinant AAV than the composition.

In one aspect, provided herein is a method of preparing a pharmaceutical composition comprising admixing a solution comprising phosphate-buffered saline and sucrose to a composition, wherein the composition comprises a recombinant adeno-associated virus (AAV) vector comprising an expression cassette encoding a transgene, and wherein the pharmaceutical composition has lower ionic strength and/or a higher level of aggregated recombinant AAV than the composition.

In one aspect, provided herein is a kit comprising: (i) a composition comprising a recombinant adeno-associated virus (AAV) vector comprising an expression cassette encoding a transgene; and (ii) a solution comprising phosphate-buffered saline and sucrose. In some embodiments, the kit further comprises instructions for admixing the composition with the solution. In some embodiments, the instructions comprise instructions on admixing the solution with the composition to obtain a pharmaceutical composition.

In some embodiments, the composition comprises a phosphate-buffered saline and sucrose. In some embodiments, the composition comprises 4% sucrose. In some embodiments, the solution comprises 10% sucrose. In some embodiments, the pharmaceutical composition has an ionic strength of about or of at most about 135 mM. In some embodiments, the pharmaceutical composition has an ionic strength of about or of at most about 40 mM. In some embodiments, the pharmaceutical composition has an ionic strength of about or of at most about 20 mM. In some embodiments, the pharmaceutical composition has substantially the same tonicity or osmolality as the composition. In some embodiments, at least some of the aggregated recombinant AAV in the pharmaceutical composition disaggregate after the pharmaceutical composition is administered to the suprachoroidal space of an eye of a human subject. In some embodiments, aggregation of the recombinant AAV is reversed to unaggregated AAV or to monomers upon suprachoroidal administration of the pharmaceutical composition. In some embodiments, the composition comprises potassium chloride, potassium phosphate monobasic, sodium chloride, sodium phosphate dibasic anhydrous, sucrose, and optionally a surfactant. In some embodiments, the composition comprises modified Dulbecco's phosphate-buffered saline solution, and optionally a surfactant. In some embodiments, the composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and a surfactant. In some embodiments, the solution comprises a phosphate-buffered sodium chloride and sucrose. In some embodiments, the solution comprises 10% Sucrose, 2.70 mM potassium chloride, 8.10 mM sodium phosphate dibasic anhydrous, 1.47 mM potassium phosphate monobasic, 292 mM sucrose, 0.001% (0.01 mg/mL) poloxamer 188, pH 7.4, and wherein the composition and solution are admixed at a composition to solution ratio of 1 to 9.

In some embodiments, admixing the solution with the composition dilutes the composition by about or at least about 2-fold, 3-fold, 4-fold, 5-fold, 6-fold, 7-fold, 8-fold, 9-fold or 10-fold. In some embodiments, admixing the solution with the composition occurs on the same day that the pharmaceutical composition is administered to the suprachoroidal space of an eye of a human subject. In some embodiments, admixing the solution with the composition occurs within 24 hours of the pharmaceutical composition being administered to the suprachoroidal space of an eye of a human subject.

In some embodiments, the pharmaceutical composition is stored prior to administration to a human subject. In some embodiments, the pharmaceutical composition is stored at about room temperature, 20° C., 4° C., or −80° C. In some embodiments, the recombinant AAV comprises components from AAV8 and the pharmaceutical composition has an ionic strength between about 30 mM to about 60 mM. In some embodiments, the recombinant AAV comprises components from AAV8 and the pharmaceutical composition has an ionic strength between about 30 mM to about 50 mM. In some embodiments, the recombinant AAV comprises components from AAV8 and the pharmaceutical composition has an ionic strength between about 20 mM to about 60 mM. In some embodiments, the recombinant AAV comprises components from AAV8 and the pharmaceutical composition has an ionic strength of about, at most about, or at least about: 5 mM, 10 mM, 15 mM, 20 mM, 25 mM, 30 mM, 35 mM, 40 mM, 45 mM, 50 mM, 55 mM, 60 mM, 65 mM, 70 mM, or higher than 70 mM. In some embodiments, the average particle diameter of the AAV is between about, at least about, or at most about: 15-70 nm, 20-60 nm, 25 nm-55 nm, 30-50 nm, or 30-70 nm (e.g., as measured by DLS). In some embodiments, the recombinant AAV comprises components from AAV9 and the pharmaceutical composition has an ionic strength between about 15 mM to about 30 mM. In some embodiments, the recombinant AAV comprises components from AAV9 and the pharmaceutical composition has an ionic strength between about 10 mM to about 50 mM. In some embodiments, the recombinant AAV comprises components from AAV9 and the pharmaceutical composition has an ionic strength between about 10 mM to about 35 mM. In some embodiments, the recombinant AAV comprises components from AAV9 and the pharmaceutical composition has an ionic strength of about, at most about, or at least about: 5 mM, 10 mM, 15 mM, 20 mM, 25 mM, 30 mM, 35 mM, 40 mM, 45 mM, 50 mM, 55 mM, 60 mM, 65 mM, 70 mM, or higher than 70 mM. In some embodiments, the recombinant AAV comprises components from AAV2 and the pharmaceutical composition has an ionic strength between about 100 mM to about 200 mM. In some embodiments, the pharmaceutical composition comprises modified Dulbecco's phosphate-buffered saline solution, and optionally a surfactant. In some embodiments, the pharmaceutical composition comprises potassium chloride, potassium phosphate monobasic, sodium chloride, sodium phosphate dibasic anhydrous, sucrose, and optionally a surfactant. In some embodiments, the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and optionally a surfactant. In some embodiments, the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4.

In one aspect provided herein is a pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4. In some embodiments, the pharmaceutical composition comprises a lower amount of AAV empty capsids as compared to a reference pharmaceutical composition. In some embodiments, the amount of the AAV empty capsids in the pharmaceutical composition is lower by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% as compared to the amount of the AAV empty capsids in the reference pharmaceutical composition. In some embodiments, the pharmaceutical composition comprises an ionic strength of about or at most about 5 mM, 10 mM, 15 mM, 20 mM, 25 mM, 30 mM, 35 mM, 40 mM, 45 mM, 50 mM, 55 mM, or 60 mM. In some embodiments, the reference pharmaceutical composition comprises an ionic strength of more than about 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, or more than about 200 mM.

In one aspect provided herein is a method of reducing or eliminating AAV empty capsids in a pharmaceutical composition, the method comprising: a. introducing a solution comprising an ionic strength of at most about 60 mM to a formulation comprising a mixture of a recombinant adeno-associated virus (AAV) vector and AAV empty capsids; and b. removing at least some of the AAV empty capsids from the formulation, wherein the formulation after step b is prepared into a pharmaceutical composition comprising the recombinant adeno-associated virus (AAV) vector, wherein the recombinant AAV vector comprises an expression cassette encoding a transgene, and wherein the pharmaceutical composition is suitable for administration to the suprachoroidal space (SCS) of an eye of a human subject. In some embodiments, the method further comprises introducing another solution comprising an ionic strength of at least about 80 mM to the formulation after step b. In some embodiments, a pharmaceutical composition is produced by the method.

In one aspect provided herein is a method of reducing or eliminating AAV empty particles in a population of AAV particles, wherein the population of AAV particles comprises empty AAV particles and AAV particles comprising an expression cassette encoding a transgene, and wherein the method comprises: a. incubating the population of AAV particles in a solution at an ionic strength of at most about 60 mM, thereby creating aggregates of the AAV particles comprising the expression cassette encoding the transgene; and b. removing at least a portion of the empty AAV particles from the population of AAV particles. In some embodiments, the method further comprises incubating the population of AAV particles after step b with another solution comprising an ionic strength of at least about 80 mM. In some embodiments, a pharmaceutical composition comprising the population of AAV particles is obtained after step b of the method.

In some embodiments, the amount of the AAV empty capsids in the formulation is reduced by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% after step b. as compared to the amount of the AAV empty capsids in the formulation prior to step a. In some embodiments, the amount of the empty AAV particles in the population of AAV particles is reduced by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% after step b. as compared to the amount of the empty AAV particles in the population of AAV particles prior to step a.

In some embodiments, the solution comprises an ionic strength of about 30 to about 50 mM. In some embodiments, the solution comprises an ionic strength of about 15 to 50 mM. In some embodiments, the solution comprises an ionic strength of at most about 50 mM. In some embodiments, the another solution comprises an ionic strength of about or at least about 150 mM.

### 2.1 ILLUSTRATIVE EMBODIMENTS

1. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical composition has an amount of viral vector aggregation such that when administered to an eye of a pig:
a. the clearance time of the pharmaceutical composition is between about 5 days and about 15 days; and
b. the thickness of the SCS at the site of injection is between about 400 μm and about 800 μm at a time within one hour of administration; and
c. the circumferential spread of the pharmaceutical composition from the site of injection is about one-eighth or less of a surface of the choroid at a time within about one hour of administration.

2. The pharmaceutical composition of paragraph 1, wherein the pharmaceutical composition comprises an ionic strength of at most about 200 mM prior to suprachoroidal administration.

3. The pharmaceutical composition of paragraph 1, wherein the pharmaceutical composition comprises at least about 3% aggregated recombinant AAV prior to suprachoroidal administration.

4. The pharmaceutical composition of any one of paragraphs 1-3, wherein the clearance time after suprachoroidal administration of the pharmaceutical composition is equal to or greater than the clearance time after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

5. The pharmaceutical composition of any one of paragraphs 1-3, wherein a circumferential spread after suprachoroidal administration of the pharmaceutical composition is smaller as compared to a circumferential spread after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

6. The pharmaceutical composition of any one of paragraphs 1-3, wherein a thickness at a site of injection after suprachoroidal administration of the pharmaceutical composition is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

7. The pharmaceutical composition of any one of paragraphs 1-3, wherein an expression level of the transgene is detected in the eye for a longer period of time after suprachoroidal administration of the pharmaceutical composition as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

8. The pharmaceutical composition of any one of paragraphs 1-3, wherein the concentration of the transgene product in the eye after suprachoroidal administration of the pharmaceutical composition is equal to or higher as compared to the concentration of the transgene product in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition;
optionally wherein the concentration of the transgene product in the back of the eye (e.g., retina) after suprachoroidal administration of the pharmaceutical composition is equal to or higher as compared to the concentration of the transgene product in the back of the eye after suprachoroidal administration of a reference pharmaceutical composition, and/or the concentration of the transgene product in the outer layer of the eye (e.g., sclera) after suprachoroidal administration of the pharmaceutical composition is lower than the concentration of the transgene product in the outer layer of the eye after suprachoroidal administration of a reference pharmaceutical composition.

9. The pharmaceutical composition of any one of paragraphs 1-3, wherein the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

10. The pharmaceutical composition of any one of paragraphs 2-9, wherein the transgene is not an anti-human vascular endothelial growth factor (anti-VEGF) antibody.

11. The pharmaceutical composition of any one of paragraphs 2-10, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

12. The pharmaceutical composition of any one of paragraphs 1-11, wherein the recombinant AAV is AAV8.

13. The pharmaceutical composition of any one of paragraphs 1-11, wherein the recombinant AAV is AAV9.

14. The pharmaceutical composition of any one of paragraphs 1-13, wherein the pharmaceutical composition has an ionic strength of about or at most about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM.

15. The pharmaceutical composition of any one of paragraphs 1-14, wherein the pharmaceutical composition comprises at least about or about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% aggregated recombinant AAV.

16. The pharmaceutical composition of any one of paragraphs 1-15, wherein the pharmaceutical composition has an ionic strength of about or of at most about 40 mM.

17. The pharmaceutical composition of any one of paragraphs 1-15, wherein the pharmaceutical composition has an ionic strength of about or of at most about 135 mM.

18. The pharmaceutical composition of any one of paragraphs 1-15, wherein the pharmaceutical composition has an ionic strength of about or of at most about 20 mM.

19. The pharmaceutical composition of any one of paragraphs 1-18, wherein the pharmaceutical composition has an average recombinant AAV diameter of about or at least about: 28 nm, 29 nm, 30 nm, 31 nm, 32 nm, 33 nm, 34 nm, 35 nm, 36 nm, 37 nm, 38 nm, 39 nm, 40 nm, 45 nm, 50 nm, 55 nm, 60 nm, 65 nm, 70 nm, 75 nm, 80 nm, 85 nm, 90 nm, 95 nm, or about or at least about 100 nm as measured by dynamic light scattering.

20. The pharmaceutical composition of any one of paragraphs 4-19, wherein the pharmaceutical composition has an average recombinant AAV diameter that is at least 2 times higher, at least 3 times higher, at least 4 times higher, at least 5 times higher, at least 6 times higher, at least 7 times higher, at least 8 times higher, at least 9 times higher, at least 10 times higher, at least 15 times higher, at least 20 times higher, at least 50 times higher, at least 100 times higher, at least 5% higher, at least 10% higher, at least 15% higher, at least 20% higher, at least 25% higher, at least 30% higher, at least 35% higher, at least 40%, at least 45% higher, at least 50% higher, at least 55% higher, at least 60% higher, at least 65% higher, at least 70% higher, at least 75% higher, at least 80% higher, at least 85% higher, at least 90% higher, at least 95% higher, at least 100% higher, at least 150% higher, or at least 200% higher, at least 250% higher, or at least 300%, at least 400% higher, or at least 500% higher than an average recombinant AAV diameter in the reference pharmaceutical composition.

21. The pharmaceutical composition of any one of paragraphs 5 and 10-20, wherein the circumferential spread after suprachoroidal administration of the pharmaceutical composition is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

22. The pharmaceutical composition of any one of paragraphs 4 and 10-21, wherein the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%.

23. The pharmaceutical composition of any one of paragraphs 1-22, wherein the clearance time after suprachoroidal administration of the pharmaceutical composition is of about 6 days to about 15 days, about 7 days to about 15 days, about 8 days to about 15 days, about 9 days to about 15 days, about 10 days to about 15 days, about 11 days to about 15 days, about 12 days to about 15 days, about 13 days to about 15 days, about 14 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, or about 5 days to about 6 days.

24. The pharmaceutical composition of any one of paragraphs 1-23, wherein the clearance time after suprachoroidal administration of the pharmaceutical composition is not prior to about 5 days, 6 days, 7 days, 8 days, 9 days, 10 days, 11 days, 12 days, 13 days, 14 days, or 15 days.

25. The pharmaceutical composition of any one of paragraphs 4-24, wherein the clearance time after suprachoroidal administration of the reference pharmaceutical composition is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.

26. The pharmaceutical composition of any one of paragraphs 1-25, wherein the clearance time is from the SCS or from the eye.

27. The pharmaceutical composition of any one of paragraphs 1-26, wherein the clearance time is the time required for the pharmaceutical composition and/or the recombinant adeno-associated virus (AAV) vector to not be detectable in the SCS by any standard method.

28. The pharmaceutical composition of any one of paragraphs 1-26, wherein the clearance time when the pharmaceutical composition and/or the recombinant adeno-associated virus (AAV) vector is present in the SCS in an amount that is at most about 2% or at most about 5% of the amount detectable by any standard method.

29. The pharmaceutical composition of any one of paragraphs 1-26, wherein the clearance time is the amount of time required following injection for the thickness at the site of injection to decrease to about 500 nm or less, about 200 nm or less, about 100 nm or less, about 50 nm or less, about 25 nm or less, about 10 nm or less, or is undetectable.

30. The pharmaceutical composition of any one of paragraphs 1-26, wherein the clearance time is the amount of time required following injection for the pharmaceutical composition to spread circumferentially from the site of injection to cover about one-sixteenth or more, about one-eighth or more, about one-fourth or more, about one-half or more, about three-fourths or more, or all of the circumference of the choroid of the eye.

31. The pharmaceutical composition of any one of paragraphs 6 and 10-30, wherein the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

32. The pharmaceutical composition of any one of paragraphs 1-31, wherein the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm.

33. The pharmaceutical composition of any one of paragraphs 1-32, wherein the thickness at the site of injection after suprachoroidal administration of the pharmaceutical composition is of at least about 50 μm, 100 μm, 200 μm, 300 μm, 400 μm, 500 μm, 600 μm, or 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

34. The pharmaceutical composition of any one of paragraphs 6 and 10-33, wherein the thickness at the site of injection after suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 μm.

35. The pharmaceutical composition of any one of paragraphs 1-34, wherein the thickness of the SCS at the site of injection is about 400 μm to about 700 μm, about 400 μm to about 600 μm, about 400 μm to about 500 μm, about 500 μm to about 800 μm, about 600 μm to about 800 μm, 700 μm to about 800 μm at a time within one hour of administration.

36. The pharmaceutical composition of paragraph 35, wherein the time within one hour of administration is within about 5 minutes of administration, within about 10 minutes of administration, within about 15 minutes of administration, within about 20 minutes of administration, within about 30 minutes of administration, within about 45 minutes of administration, or within about 60 minutes of administration.

37. The pharmaceutical composition of any one of paragraphs 1-36, wherein the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

38. The pharmaceutical composition of any one of paragraphs 1-36, wherein the circumferential spread of the pharmaceutical composition from the site of injection is about one-eighth or less of a surface of the choroid at a time within about 5 minutes of administration, within about 10 minutes of administration, within about 15 minutes of administration, within about 20 minutes of administration, within about 30 minutes of administration, within about 45 minutes of administration, or within about 60 minutes of administration.

39. The pharmaceutical composition of paragraph 38, wherein the circumferential spread from the site of injection is about one-sixteenth or less of a surface of the choroid.

40. The pharmaceutical composition of any one of paragraphs 8 and 10-39, wherein the concentration of the transgene product in the eye after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

41. The pharmaceutical composition of any one of paragraphs 7 and 10-40, wherein the longer period of time after suprachoroidal administration of the pharmaceutical composition is longer by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

42. The pharmaceutical composition of any one of paragraphs 1-41, wherein the transgene is detected in the eye after suprachoroidal administration of the pharmaceutical composition for at least about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

43. The pharmaceutical composition of any one of paragraphs 4-42, wherein the transgene is detected in the eye after suprachoroidal administration of the reference pharmaceutical composition for at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

44. The pharmaceutical composition of any one of paragraphs 9 and 10-43, wherein the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

45. The pharmaceutical composition of any one of paragraphs 2-44, wherein the recombinant AAV stability in the pharmaceutical composition is at least about 50% of the recombinant AAV stability in the reference pharmaceutical composition.

46. The pharmaceutical composition of paragraph 45, wherein the recombinant AAV stability is determined by infectivity of the recombinant AAV.

47. The pharmaceutical composition of paragraph 45, wherein the recombinant AAV stability is determined by a level of free DNA released by the recombinant AAV.

48. The pharmaceutical composition of paragraph 47, wherein the pharmaceutical composition comprises at least about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less free DNA as compared to a level of free DNA in the reference pharmaceutical composition.

49. The pharmaceutical composition of paragraph 46, wherein the recombinant AAV in the pharmaceutical composition has an infectivity that is about 50% lower, about the same, or at least about 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times higher as compared to the infectivity of the recombinant AAV in the reference pharmaceutical composition.

50. The pharmaceutical composition of any one of paragraphs 1-49, wherein the transgene is a transgene suitable to treat, or otherwise ameliorate, prevent or slow the progression of a disease of interest.

51. The pharmaceutical composition of any one of paragraphs 1-50, wherein the human subject is diagnosed with nAMD (wet AMD), dry AMD, retinal vein occlusion (RVO), diabetic macular edema (DME), or diabetic retinopathy (DR), or Batten disease.

52. The pharmaceutical composition of any one of paragraphs 1-50, wherein the human subject is diagnosed with glaucoma, non-infectious uveitis, or kallikrein-related disease.

53. The pharmaceutical composition of any one of paragraphs 1-3, 4-9, and 10-52, wherein the AAV encodes Palmitoyl-Protein Thioesterase 1 (PPT1), Tripeptidyl-Peptidase 1 (TPP1), anti-TNF fusion protein, anti-kallikrein antibody or antigen-binding fragment, anti-TNF antibody or antigen-binding fragment, anti-C3 antibody or antigen-binding fragment, or anti-C5 antibody or antigen-binding fragment.

54. The pharmaceutical composition of any one of paragraphs 4-53, wherein the amount of the recombinant AAV genome copies is based on a vector genome concentration.

55. The pharmaceutical composition of any one of paragraphs 4-53, wherein the amount of the recombinant AAV genome copies is based on genome copies per administration.

56. The pharmaceutical composition of any one of paragraphs 4-53, wherein the amount of the recombinant AAV genome copies is based on total genome copies administered to the human subject.

57. The pharmaceutical composition of paragraph 55, wherein the genome copies per administration is the genome copies of the recombinant AAV per suprachoroidal administration.

58. The pharmaceutical composition of paragraph 56, wherein the total genome copies administered is the total genome copies of the recombinant AAV administered suprachoroidally.

59. The pharmaceutical composition of paragraph 54, wherein the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 6×10¹² GC/mL, about 1.5×10¹³ GC/mL, about 2×10¹³ GC/mL, or about 3×10¹³ GC/mL.

60. The pharmaceutical composition of any one of paragraphs 56 and 58, wherein the total number of genome copies administered is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

61. The pharmaceutical composition of any one of paragraphs 55 and 57, wherein the total number of genome copies per administration is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

62. The pharmaceutical composition of any one of paragraphs 2-61, wherein the pharmaceutical composition is administered once, twice, three times, four times, five times, six times, seven times, eight times, nine times, ten times, fifteen times, twenty times, twenty five times, or thirty times.

63. The pharmaceutical composition of any one of paragraphs 4-62, wherein the reference pharmaceutical composition is administered once, twice, three times, four times, five times, six times, seven times, eight times, nine times, ten times, fifteen times, twenty times, twenty-five times, or thirty times.

64. The pharmaceutical composition of any one of paragraphs 2-63, wherein the pharmaceutical composition is administered once in one day, twice in one day, three times in one day, four times in one day, five times in one day, six times in one day, or seven times in one day.

65. The pharmaceutical composition of any one of paragraphs 4-63, wherein the reference pharmaceutical composition is administered once in one day, twice in one day, three times in one day, four times in one day, five times in one day, six times in one day, or seven times in one day.

66. The pharmaceutical composition of any one of paragraphs 2-65, wherein the reference pharmaceutical composition comprises DPBS and sucrose.

67. A method of preparing a pharmaceutical composition comprising:
a. preparing a composition comprising phosphate-buffered saline, sucrose, and a recombinant adeno-associated virus (AAV) vector comprising an expression cassette encoding a transgene; and
b. admixing a solution comprising phosphate-buffered saline and sucrose to the composition,
   wherein the pharmaceutical composition has lower ionic strength and/or a higher level of aggregated recombinant AAV than the composition.

68. A method of preparing a pharmaceutical composition comprising admixing a solution comprising phosphate-buffered saline and sucrose to a composition, wherein the composition comprises a recombinant adeno-associated virus (AAV) vector comprising an expression cassette encoding a transgene, and wherein the pharmaceutical composition has lower ionic strength and/or a higher level of aggregated recombinant AAV than the composition.

69. A kit comprising:
a. a composition comprising a recombinant adeno-associated virus (AAV) vector comprising an expression cassette encoding a transgene; and
b. a solution comprising phosphate-buffered saline and sucrose.

70. The kit of paragraph 69, wherein the kit further comprises instructions for admixing the composition with the solution.

71. The method of paragraph 68 or the kit of paragraph 69, wherein the composition comprises a phosphate-buffered saline and sucrose.

72. The method of any one of paragraphs 67 and 71, or the kit of any one of paragraphs 69 and 71, wherein the composition comprises 4% sucrose.

73. The method of any one of paragraphs 67, 68, 71 and 72, or the kit of any one of paragraphs 69-72, wherein the solution comprises 10% sucrose.

74. The method of any one of paragraphs 67, 68, and 71-73, or the pharmaceutical composition of any one of paragraphs 1-66, wherein the pharmaceutical composition has an ionic strength of about or of at most about 135 mM.

75. The method of any one of paragraphs 67, 68, and 71-74, or the pharmaceutical composition of any one of paragraphs 1-66, wherein the pharmaceutical composition has an ionic strength of about or of at most about 40 mM.

76. The method of any one of paragraphs 67, 68, and 71-75, or the pharmaceutical composition of any one of paragraphs 1-66, wherein the pharmaceutical composition has an ionic strength of about or of at most about 20 mM.

77. The method of any one of paragraphs 67, 68, and 71-76, or the pharmaceutical composition of any one of paragraphs 1-66, wherein the pharmaceutical composition has substantially the same tonicity or osmolality as the composition.

78. The method of any one of paragraphs 67, 68, and 71-77, or the pharmaceutical composition of any one of paragraphs 1-66, wherein at least some of the aggregated recombinant AAV in the pharmaceutical composition disaggregate after the pharmaceutical composition is administered to the suprachoroidal space of an eye of a human subject.

79. The method of any one of paragraphs 67, 68, and 71-78, or the pharmaceutical composition of any one of paragraphs 1-66, wherein aggregation of the recombinant AAV is reversed to unaggregated AAV or to monomers upon suprachoroidal administration of the pharmaceutical composition.

80. The method of any one of paragraphs 67, 68, and 71-79, or the kit of any one of paragraphs 69-72, wherein the composition comprises potassium chloride, potassium phosphate monobasic, sodium chloride, sodium phosphate dibasic anhydrous, sucrose, and optionally a surfactant.

81. The method of any one of paragraphs 67, 68, and 71-80, or the kit of any one of paragraphs 69-72 and 80, wherein the composition comprises modified Dulbecco's phosphate-buffered saline solution, and optionally a surfactant.

82. The method of any one of paragraphs 67, 68, and 71-81, or the kit of any one of paragraphs 69-72 and 80-81, wherein the composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and a surfactant.

83. The method of any one of paragraphs 67, 68, and 71-82, or the kit of any one of paragraphs 69-72 and 80-82, wherein the solution comprises a phosphate-buffered sodium chloride and sucrose.

84. The kit of paragraph 70, wherein the instructions comprise instructions on admixing the solution with the composition to obtain a pharmaceutical composition.

85. The method of any one of paragraphs 67, 68, and 71-83, or the kit of paragraph 84, wherein the admixing the solution with the composition dilutes the composition by about or at least about 2-fold, 3-fold, 4-fold, 5-fold, 6-fold, 7-fold, 8-fold, 9-fold or 10-fold.

86. The method of any one of paragraphs 67, 68, 71-83 and 85, or the kit of any one of paragraphs 84-85, wherein the admixing the solution with the composition occurs on the same day that the pharmaceutical composition is administered to the suprachoroidal space of an eye of a human subject.

87. The method of any one of paragraphs 67, 68, 71-83 and 85-86, or the kit of any one of paragraphs 84-86, wherein the admixing the solution with the composition occurs within 24 hours of the pharmaceutical composition being administered to the suprachoroidal space of an eye of a human subject.

88. The method of any one of paragraphs 67, 68, 71-83 and 85-87, or the kit of any one of paragraphs 84-87, or the pharmaceutical composition of any one of paragraphs 2-66 and 74-79, wherein the pharmaceutical composition is stored prior to administration to a human subject.

89. The method of any one of paragraphs 67, 68, 71-83 and 85-88, or the kit of any one of paragraphs 84-88, or the pharmaceutical composition of any one of paragraphs 2-66, 74-79, and 88, wherein the pharmaceutical composition is stored at about room temperature, 20° C., 4° C., or −80° C.

90. The method of any one of paragraphs 67, 68, 71-83 and 85-89, or the kit of any one of paragraphs 84-89, or the pharmaceutical composition of any one of paragraphs 2-66, 74-79, and 88-89, wherein the pharmaceutical composition comprises about 1.0×10¹² to about 3.0×10¹² genome copies of the recombinant AAV.

91. The method of any one of paragraphs 67, 68, 71-83 and 85-90, or the kit of any one of paragraphs 84-90, wherein the recombinant AAV comprises components from one or more adeno-associated virus serotypes selected from AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

92. The method of any one of paragraphs 67, 68, 71-83 and 85-91, or the kit of any one of paragraphs 84-91, or the pharmaceutical composition of any one of paragraphs 2-66, 74-79, and 88-90, wherein the recombinant AAV comprises components from AAV8 and the pharmaceutical composition has an ionic strength between about 30 mM to about 60 mM.

93. The method of any one of paragraphs 67, 68, 71-83 and 85-91, or the kit of any one of paragraphs 84-91, or the pharmaceutical composition of any one of paragraphs 2-66, 74-79, and 88-90, wherein the recombinant AAV comprises components from AAV9 and the pharmaceutical composition has an ionic strength between about 15 mM to about 30 mM.

94. The method of any one of paragraphs 67, 68, 71-83 and 85-91, or the kit of any one of paragraphs 84-91, or the pharmaceutical composition of any one of paragraphs 1-66, 74-79, and 88-90, wherein the recombinant AAV comprises components from AAV2 and the pharmaceutical composition has an ionic strength between about 100 mM to about 200 mM.

95. The pharmaceutical composition of any one of paragraphs 1-66, 74-79, and 88-94, wherein the pharmaceutical composition comprises modified Dulbecco's phosphate-buffered saline solution, and optionally a surfactant.

96. The pharmaceutical composition of any one of paragraphs 1-66, 74-79, and 88-95, wherein the pharmaceutical composition comprises potassium chloride, potassium phosphate monobasic, sodium chloride, sodium phosphate dibasic anhydrous, sucrose, and optionally a surfactant.

97. The pharmaceutical composition of any one of paragraphs 1-66, 74-79, and 88-96, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and optionally a surfactant.

98. The pharmaceutical composition of any one of paragraphs 1-66, 74-79, and 88-96, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4.

99. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4.

100. The pharmaceutical composition of any one of paragraphs 1-66, 74-79, and 88-99, wherein the pharmaceutical composition comprises a lower amount of AAV empty capsids as compared to a reference pharmaceutical composition.

101. The pharmaceutical composition of paragraph 100, wherein the amount of the AAV empty capsids in the pharmaceutical composition is lower by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% as compared to the amount of the AAV empty capsids in the reference pharmaceutical composition.

102. The pharmaceutical composition of any one of paragraphs 1-66, 74-79, and 88-101, wherein the pharmaceutical composition comprises an ionic strength of about or at most about 5 mM, 10 mM, 15 mM, 20 mM, 25 mM, 30 mM, 35 mM, 40 mM, 45 mM, 50 mM, 55 mM, or 60 mM.

103. The pharmaceutical composition of any one of paragraphs 100-102, wherein the reference pharmaceutical composition comprises an ionic strength of more than about 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, or more than about 200 mM.

104. A method of reducing or eliminating AAV empty capsids in a pharmaceutical composition, the method comprising:
a. introducing a solution comprising an ionic strength of at most about 60 mM to a formulation comprising a mixture of a recombinant adeno-associated virus (AAV) vector and AAV empty capsids; and
b. removing at least some of the AAV empty capsids from the formulation, wherein the formulation after step b is prepared into a pharmaceutical composition comprising the recombinant adeno-associated virus (AAV) vector, wherein the recombinant AAV vector comprises an expression cassette encoding a transgene, and wherein the pharmaceutical composition is suitable for administration to the suprachoroidal space (SCS) of an eye of a human subject.

105. A method of reducing or eliminating AAV empty particles in a population of AAV particles, wherein the population of AAV particles comprises empty AAV particles and AAV particles comprising an expression cassette encoding a transgene, and wherein the method comprises:
a. incubating the population of AAV particles in a solution at an ionic strength of at most about 60 mM, thereby creating aggregates of the AAV particles comprising the expression cassette encoding the transgene; and
b. removing at least a portion of the empty AAV particles from the population of AAV particles.

106. The method of paragraph 104, wherein the method further comprises introducing another solution comprising an ionic strength of at least about 80 mM to the formulation after step b.

107. The method of paragraph 105, wherein the method further comprises incubating the population of AAV particles after step b with another solution comprising an ionic strength of at least about 80 mM.

108. The method of paragraph 104 or 106, wherein the amount of the AAV empty capsids in the formulation is reduced by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% after step b. as compared to the amount of the AAV empty capsids in the formulation prior to step a.

109. The method of paragraph 105 or 107, wherein the amount of the empty AAV particles in the population of AAV particles is reduced by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% after step b. as compared to the amount of the empty AAV particles in the population of AAV particles prior to step a.

110. The method of any one of paragraphs 104-109, wherein the solution comprises an ionic strength of about 30 to about 50 mM.

111. The method of any one of paragraphs 104-109, wherein the solution comprises an ionic strength of about 15 to 50 mM.

112. The method of any one of paragraphs 104-109, wherein the solution comprises an ionic strength of at most about 50 mM.

113. The method of any one of paragraphs 104-109, wherein the another solution comprises an ionic strength of about or at least about 150 mM.

114. A pharmaceutical composition produced by the method of any one of paragraphs 104, 106, and 108-113.

115. A pharmaceutical composition comprising the population of AAV particles obtained after step b of the method of any one of paragraphs 105 and 107-113.

116. The method or the kit of paragraph 81 or 82, wherein the solution comprises 10% Sucrose, 2.70 mM potassium chloride, 8.10 mM sodium phosphate dibasic anhydrous, 1.47 mM potassium phosphate monobasic, 292 mM sucrose, 0.001% (0.01 mg/mL) poloxamer 188, pH 7.4, and wherein the composition and solution are admixed at a composition to solution ratio of 1 to 9.

### 3. BRIEF DESCRIPTION OF THE DRAWINGS

FIG. 1. Overview of induced clustering of AAV capsids using a low salt and/or low ionic strength diluent.

FIG. 2. Graph showing impact of ionic strength and salt concentration on diameter of AAV.

FIGS. 3A-3B. Electron microscopy visual representation of the impact of ionic strength and salt concentration on diameter of AAV. FIG. 3A shows AAVs in a control or reference pharmaceutical composition. FIG. 3A shows that AAVs do not aggregate in a reference pharmaceutical composition. FIG. 3B shows AAV aggregation in low ionic strength solutions (pharmaceutical compositions comprising lower ionic strength as compared to the reference pharmaceutical composition).

FIG. 4. Graph showing average apparent diameter of AAV clusters in solutions containing different ionic strengths and salt amounts as measured from the time of dilution to about 21 hours post dilution, at 25° C. The control comprises recombinant AAV in modified DPBS with 4% sucrose. The two-times, four-times, and eight-times dilution comprises recombinant AAV in modified DPBS with 4% sucrose diluted with different amounts of phosphate-buffered 10% sucrose diluent to obtain the two-times, four-times, and eight-times dilutions. Clusters were stable at 25° C. for at least 21 hours. At around 21.8 hours a spike of sodium chloride was added to obtain solutions with at least 75 mM salt level, which reversed the clusters back to monomers.

FIG. 5. Graph showing cumulants dynamic light scattering (DLS) intensity-weighted average apparent diameter for recombinant AAV. FIG. 5 shows the initial induced clustering data from FIG. 4 up to about 21.8 hours of AAV in the control, in the two-times, four-times, and eight-times dilutions into low ionic strength phosphate-buffered 10% sucrose diluent (refer to FIG. 4).

FIG. 6. Graph showing cumulants dynamic light scattering (DLS) intensity-weighted average apparent diameter for recombinant AAV. FIG. 6 shows the reversal of the induced clustering, in the two-times, four-times, and eight-times dilutions into low ionic strength phosphate-buffered 10% sucrose diluent. Reversal of induced clustering was effected with a spike of salt at about 21.8 hours of AAV (refer to FIG. 4).

FIG. 7. Graph showing cumulants diameter of recombinant AAV in modified DPBS with 4% sucrose formulation, in an induced-clustering low-salt solution (a ten-times dilution) prepared by dilution with phosphate-buffered 10% sucrose diluent, and after reversal of the clustering with addition of salt. The figure also shows that clustering remained after the samples were heated to 37° C.

FIGS. 8A-8B. Impact of ionic strength on AAV8 in F3 and AAV9 in F4 colloidal stability. FIG. 8A: AAV8 empty capsids (circle), AAV8 lot 1 (square). FIG. 8B: AAV9 empty capsids (circle), AAV9 lot 1 (square).

FIG. 9. Illustration of a representative purification of AAV (e.g. with HF-TFF) based on the colloidal stability difference between empty and full capsids under low ionic strength conditions.

FIG. 10. UV absorption comparison between DNA, empty capsid, and AAV full capsid, utilized to assess empty vs. full capsids in a composition.

### 4. DETAILED DESCRIPTION OF THE INVENTION

Provided herein are pharmaceutical compositions comprising recombinant adeno-associated virus (AAV) vector comprising an expression cassette encoding a transgene suitable for administration to a suprachoroidal space (SCS) of an eye of a subject. The subject can be a subject diagnosed with one of more diseases described in Section 4.5. The AAV vectors are described in Section 4.4 and dosages of such vectors are described in Section 4.3. In some embodiments, pharmaceutical compositions provided in Section 4.1 are formulated such that they have one or more functional properties described in Section 4.2. In certain embodiments, the pharmaceutical composition provided herein has various advantages, for example, increased or slower clearance time (Section 4.2.1); decreased circumferential spread (Section 4.2.2); increased SCS thickness (Section 4.2.3); increased AAV level and rate of transduction (rate of infection) at the site of injection (Section 4.2.4); and increased concentration of the transgene after the pharmaceutical composition is administered in the SCS. Without being bound by theory, the functional properties can be achieved using compositions comprising aggregated viral vectors formulations as disclosed in Section 4.1. Also provided herein are assays that may be used in related studies (Section 4.6).

### 4.1 Formulation of Pharmaceutical Composition

The disclosure provides a pharmaceutical composition suitable for suprachoroidal administration comprising a recombinant adeno-associated virus (AAV) vector comprising an expression cassette encoding a transgene. In some embodiments, several pharmaceutical compositions (e.g., diluted formulation or lower ionic strength formulation) having different aggregation levels of AAV are used to administer an AAV encoding a transgene.

In some embodiments, the pharmaceutical composition has a higher level of AAV aggregation than a comparable pharmaceutical composition (a reference pharmaceutical composition). In some embodiments, the pharmaceutical composition and the reference pharmaceutical composition comprise a recombinant adeno-associated virus (AAV) vector comprising an expression cassette encoding a transgene. In some embodiments, the pharmaceutical composition and the reference pharmaceutical composition have the same vector genome concentration. In some embodiments, the pharmaceutical composition and a reference pharmaceutical composition have the same amount of genome copies. In some embodiments, the pharmaceutical composition and a reference pharmaceutical composition are each administered to a subject using the same amount of genome copies. In some embodiments, the pharmaceutical composition has a percentage of aggregated viral vectors that is higher than the percentage of viral vector aggregation of a control. In some embodiments, the pharmaceutical composition has a percentage of aggregated viral vectors that is higher than the percentage of aggregated viral vectors of a solution normally used for subretinal injection. In some embodiments, the reference pharmaceutical composition has less viral vector aggregation than the pharmaceutical composition. In some embodiments, the reference pharmaceutical composition has the same or similar percentage of viral vector aggregation than the pharmaceutical composition. In some embodiments, the reference pharmaceutical composition is a control solution (e.g., DPBS, PBS, water, or HBSS). In some embodiments, the reference pharmaceutical composition comprises the AAV in a control solution (e.g., DPBS, PBS, water, or HBSS). In some embodiments, the reference pharmaceutical composition comprises sucrose. In some embodiments, the reference pharmaceutical composition is a pharmaceutical composition commonly used for AAV subretinal injection.

In some embodiments, the pharmaceutical composition comprising the AAV is diluted so that the AAV in the pharmaceutical composition forms clustering or aggregation of AAV. In some embodiments, the pharmaceutical composition is diluted with any solution suitable to provide AAV solutions containing lower ionic strength and salt content. In some embodiments, the pharmaceutical composition is diluted with any solution suitable to reduce the ionic strength of the pharmaceutical composition. In some embodiments, the pharmaceutical composition is diluted with a solution having reduced ionic excipient sodium chloride to induce AAV clustering. In some embodiments, the pharmaceutical composition is diluted with a solution containing reduced ionic excipient and/or increased non-ionic excipient. In some embodiments, the pharmaceutical composition is diluted with a solution containing reduced ionic excipient sodium chloride and increased non-ionic excipient sucrose. In some embodiments, the pharmaceutical composition is diluted with phosphate-buffered 10% sucrose solutions. In some embodiments, the pharmaceutical composition is diluted with solutions comprising varying non-ionic excipient levels (e.g., 4% sucrose, 6% sucrose, 8% sucrose, 10% sucrose, 15% sucrose, or 20% sucrose). In some embodiments, the pharmaceutical composition is diluted with a solution comprising 4% sucrose, 6% sucrose, or 10% sucrose. In some embodiments, the pharmaceutical composition is diluted with solutions comprising varying ionic excipient levels (e.g., a solution containing reduced sodium chloride concentration as compared to the sodium chloride concentration in the pharmaceutical composition). In some embodiments, the pharmaceutical composition has the same tonicity/osmolality before and after dilution. In some embodiments, the pharmaceutical composition has the same tonicity/osmolality as the reference pharmaceutical composition or a control. In some embodiments, the pharmaceutical composition has a higher tonicity/osmolality than a reference pharmaceutical composition or a control. In some embodiments, the pharmaceutical composition has a lower tonicity/osmolality than a reference pharmaceutical composition or a control. In some embodiments, the pharmaceutical composition has a tonicity/osmolality equal to or greater than 240 mOsm/kg. In some embodiments, the pharmaceutical composition or the reference pharmaceutical composition has a tonicity/osmolality that is at least 240 mOsm/kg.

In some embodiments, the pharmaceutical composition (e.g., diluted formulation or lower ionic strength formulation) is diluted before administration (e.g., suprachoroidal administration). In some embodiments, the pharmaceutical composition is diluted on the same day as the administration (e.g., suprachoroidal administration). In some embodiments, the pharmaceutical composition is diluted about 20 hours or about 24 hours before the administration (e.g., suprachoroidal administration). In some embodiments, the pharmaceutical composition is diluted about 5 minutes, 10 minutes, 15 minutes, 30 minutes, 45 minutes, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, two weeks, three weeks, four weeks, two months, three months, four months, five months, six months, seven months, eight months, nine months, ten months, eleven months, twelve months, two years, three years, four years, five years, ten years, fifteen years, twenty years (or longer) before administration. In some embodiments, the pharmaceutical composition is diluted at most about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, two weeks, three weeks, four weeks, two months, three months, four months, five months, six months, seven months, eight months, nine months, ten months, eleven months, twelve months, two years, three years, four years, five years, ten years, fifteen years, twenty years (or longer) before administration.

In some embodiments, the pharmaceutical composition is diluted and kept at room temperature (e.g., after dilution or prior to administration). In some embodiments, the pharmaceutical composition is diluted and kept at about 20° C. after the dilution. In some embodiments, the pharmaceutical composition is diluted and kept at about 25° C. after the dilution. In some embodiments, the pharmaceutical composition is diluted and kept at 4° C. after the dilution. In some embodiments, the pharmaceutical composition is diluted and kept at −20° C. after the dilution. In some embodiments, the pharmaceutical composition is diluted and kept at −80° C. after the dilution. In some embodiments, the pharmaceutical composition is diluted and is flash frozen after the dilution. In some embodiments, the undiluted pharmaceutical composition, the reference pharmaceutical composition, the diluted pharmaceutical composition, and/or the diluted reference pharmaceutical composition are suitable for long-term storage (e.g., at an appropriate temperature). In some embodiments, long-term storage is about or at least about 1 month, 2 months, 3 months, 6 months, 9 months, 1 year, 2 years, 3 years, 4 years, 5 years, or longer than 5 years.

In some embodiments, the pharmaceutical composition (e.g., diluted pharmaceutical composition) or the diluted reference pharmaceutical composition has an ionic strength (or contains an ionic excipient concentration) that is about or at most about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, 300 mM, 350 mM, 400 mM, 450 mM, 500 mM, 550 mM, 600 mM, 650 mM, 700 mM, 800 mM, 900 mM, or at most about 1000 mM. In some embodiments, the pharmaceutical composition (e.g., diluted pharmaceutical composition) or the diluted reference pharmaceutical composition has an ionic strength (or contains an ionic excipient concentration) that is about or at most about 15 mM, 20 mM, 40 mM, 60 mM, 80 mM, 100 mM, 130 mM, 150 mM, 175 mM, 200 mM, or 300 mM. In some embodiments, the pharmaceutical composition (e.g., diluted pharmaceutical composition) or the diluted reference pharmaceutical composition has an ionic strength (or contains an ionic excipient concentration) in the range of about 5 mM to about 140 mM, of about 15 mM to about 150 mM, of about 5 mM to about 65 mM, of about 5 mM to about 200 mM, of about 15 mM to about 134 mM, of about 10 mM to about 200 mM, of about 20 mM to about 45 mM, of about 15 mM to about 300 mM, of about 5 mM to about 600 mM, of about 15 mM to about 600 mM, of about 10 mM to about 550 mM, or of about 15 mM to about 70 mM. In some embodiments, or the diluted reference pharmaceutical composition has an ionic strength (or contains an ionic excipient concentration) that is at most about 40 mM. In some embodiments, the pharmaceutical composition (e.g., diluted pharmaceutical composition) or the diluted reference pharmaceutical composition has an ionic strength (or contains an ionic excipient concentration) that is at most about 20 mM. In some embodiments, the pharmaceutical composition (e.g., diluted pharmaceutical composition) or the diluted reference pharmaceutical composition has an ionic strength (or contains an ionic excipient concentration) that is at most about 100 mM. In some embodiments, the pharmaceutical composition (e.g., diluted pharmaceutical composition) or the diluted reference pharmaceutical composition has an ionic strength (or contains an ionic excipient concentration) that is at most about 200 mM. In some embodiments, the reference pharmaceutical composition or the undiluted pharmaceutical composition has an ionic strength that is at least about or about 80 mM, 100 mM, 120 mM, 150 mM, 200 mM, or higher than 200 mM.

In some embodiments, the pharmaceutical composition (e.g., diluted pharmaceutical composition) has a lower ionic strength than the undiluted pharmaceutical composition prior to dilution. In some embodiments, the pharmaceutical composition is diluted about or at most about two-times, three-times, four-times, five-times, six-times, seven-times, eight-times, nine-times, ten-times, fifteen-times, twenty-times, thirty-times, forty-times, fifty-times, or one hundred-times. In some embodiments, the pharmaceutical composition (e.g., diluted pharmaceutical composition) or the diluted reference pharmaceutical composition has an ionic strength that is below the ionic strength necessary to induce clustering of a viral vector (e.g., clustering threshold). In some embodiments, the pharmaceutical composition (e.g., diluted pharmaceutical composition) or the diluted reference pharmaceutical composition has an ionic strength that is below the ionic strength for AAV clustering. In some embodiments, the pharmaceutical composition (e.g., diluted pharmaceutical composition) or the diluted reference pharmaceutical composition has an ionic strength that is two-times below the ionic strength for AAV clustering. In some embodiments, the clustering threshold for a viral vector is determined. In some embodiments, the clustering threshold is determined by any suitable method or any suitable assay disclosed in Section 4.6. In some embodiments, the clustering threshold for AAV8 correlates an ionic strength of about 60 mM. In some embodiments, the pharmaceutical composition (e.g., diluted pharmaceutical composition) has an ionic strength that is about half the ionic strength for clustering threshold (e.g., 30 mM). In some embodiments, the pharmaceutical composition (e.g., diluted pharmaceutical composition) has an ionic strength that is about or at most about 30 mM. In some embodiments, the pharmaceutical composition (e.g., diluted pharmaceutical composition) has an ionic strength that is about two-thirds the ionic strength for clustering threshold (e.g., 40 mM). In some embodiments, the pharmaceutical composition (e.g., diluted pharmaceutical composition) has an ionic strength that is about or at most about 40 mM. In some embodiments, the clustering threshold for AAV9 correlates to an ionic strength of about 30 mM. In some embodiments, the clustering threshold for AAV2 correlates to an ionic strength of about 200 mM. In some embodiments, the pharmaceutical composition (e.g., diluted pharmaceutical composition) has an ionic strength that is about half the ionic strength for clustering threshold (e.g., 15 mM or 100 mM). In some embodiments, the pharmaceutical composition (e.g., diluted pharmaceutical composition) has an ionic strength that is about two-thirds the ionic strength for clustering threshold (e.g., 20 mM or 134 mM). In some embodiments, the pharmaceutical composition (e.g., diluted pharmaceutical composition) has an ionic strength that is about or at most about 20 mM. In some embodiments, the pharmaceutical composition (e.g., diluted pharmaceutical composition) has an ionic strength that is about or at most about 134 mM. In some embodiments, the pharmaceutical composition (e.g., diluted pharmaceutical composition) has an ionic strength that is about three-fourths the ionic strength for clustering threshold. In some embodiments, the pharmaceutical composition (e.g., diluted pharmaceutical composition) has an ionic strength that is about two-fifths the ionic strength for clustering threshold.

In some embodiments, the pharmaceutical composition (e.g., diluted pharmaceutical composition) or the diluted reference pharmaceutical composition has an ionic strength that is about or at most 2%, 5%, 10%, 15%, 20%, 25%, 30%, 35%, 40%, 45%, 50%, 55%, 60%, 65%, 70%, 75%, 80%, 85%, 90%, or 95% the ionic strength for clustering threshold.

In some embodiments, the pharmaceutical composition (e.g., diluted pharmaceutical composition) or the diluted reference pharmaceutical composition has a salt concentration (e.g., sodium chloride) that is about or at most about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, 300 mM, 350 mM, 400 mM, 450 mM, 500 mM, 550 mM, 600 mM, 650 mM, 700 mM, 800 mM, 900 mM, or at most about 1000 mM. In some embodiments, the pharmaceutical composition (e.g., diluted pharmaceutical composition) or the diluted reference pharmaceutical composition has a salt concentration (e.g., sodium chloride) that is about or at most about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 200 mM, or 300 mM. In some embodiments, the pharmaceutical composition (e.g., diluted pharmaceutical composition) or the diluted reference pharmaceutical composition has a salt concentration (e.g., sodium chloride) that is about or at most about 10 mM, 20 mM, 40 mM, 60 mM, 100 mM, or 150 mM. In some embodiments, the reference pharmaceutical composition or an undiluted pharmaceutical composition comprises a salt (e.g., sodium chloride) concentration of at about or at least about 80 mM, 100 mM, 120 mM 150 mM, 175 mM, 200 mM or higher.

In some embodiments, the pharmaceutical composition (e.g., diluted pharmaceutical composition) or the diluted reference pharmaceutical composition has a tonicity/osmolality that is at least about 240 mOsm/kg. In some embodiments, the pharmaceutical composition (e.g., diluted pharmaceutical composition) or the diluted reference pharmaceutical composition has a tonicity/osmolality that is at least about 100 mOsm/kg, 150 mOsm/kg, 160 mOsm/kg, 170 mOsm/kg, 180 mOsm/kg, 190 mOsm/kg, 200 mOsm/kg, 210 mOsm/kg, 220 mOsm/kg, 230 mOsm/kg, 240 mOsm/kg, 250 mOsm/kg, 260 mOsm/kg, 270 mOsm/kg, 280 mOsm/kg, 290 mOsm/kg, 300 mOsm/kg, 310 mOsm/kg, 320 mOsm/kg, 340 mOsm/kg, 350 mOsm/kg, 360 mOsm/kg, 370 mOsm/kg, 380 mOsm/kg, 390 mOsm/kg, 400 mOsm/kg, 450 mOsm/kg, 500 mOsm/kg, 550 mOsm/kg, 600 mOsm/kg, 650 mOsm/kg, or 700 mOsm/kg. In some embodiments, the pharmaceutical composition (e.g., diluted pharmaceutical composition) or the diluted reference pharmaceutical composition has a tonicity/osmolality that is at least about 240 mOsm/kg to about 600 mOsm/kg, 240 mOsm/kg to about 350 mOsm/kg, at least about 220 mOsm/kg to about 400 mOsm/kg, or at least about 200 mOsm/kg to about 500 mOsm/kg. In some embodiments, the pharmaceutical composition (e.g., diluted pharmaceutical composition) or the diluted reference pharmaceutical composition has a tonicity/osmolality that is at between about 240 mOsm/kg to about 600 mOsm/kg.

In some embodiments, the pharmaceutical composition (e.g., diluted pharmaceutical composition) or the diluted reference pharmaceutical composition comprises at least some aggregated viral vectors. In some embodiments, the pharmaceutical composition (e.g., diluted pharmaceutical composition) or the diluted reference pharmaceutical composition comprises about or at least about 1%, 2%, 3%, 4%, 5%, 6%, 7%, 8%, 9%, 10%, 11%, 12% 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% aggregated viral vectors (e.g., aggregated AAV). In some embodiments, the pharmaceutical composition (e.g., diluted pharmaceutical composition) or the diluted reference pharmaceutical composition comprises from about 1% to about 20%, 1% to about 10%, 1% to about 50%, 3% to about 95%, 3% to about 90%, 3% to about 80%, 3% to about 70%, 3% to about 60%, 3% to about 50%, 3% to about 40%, 3% to about 30%, 3% to about 20%, 3% to about 15%, 3% to about 10%, 5% to about 95%, 5% to about 90%, 5% to about 80%, 5% to about 70%, 5% to about 60%, 5% to about 50%, 5% to about 40%, 5% to about 30%, 5% to about 20%, 5% to about 15%, 5% to about 10%, 10% to about 95%, 10% to about 90%, 10% to about 80%, 10% to about 70%, 10% to about 60%, 10% to about 50%, 10% to about 40%, 10% to about 30%, 10% to about 20%, 10% to about 15%, 15% to about 95%, 15% to about 90%, 15% to about 80%, 15% to about 70%, 15% to about 60%, 15% to about 50%, 15% to about 40%, 15% to about 30%, 15% to about 20%, 20% to about 95%, 20% to about 90%, 20% to about 80%, 20% to about 70%, 20% to about 60%, 20% to about 50%, 20% to about 40%, 20% to about 30%, 30% to about 95%, 30% to about 90%, 30% to about 80%, 30% to about 70%, 30% to about 60%, 30% to about 50%, 30% to about 40%, 40% to about 95%, 40% to about 90%, 40% to about 80%, 40% to about 70%, 40% to about 60%, or 40% to about 50% aggregated viral vectors (e.g., aggregated AAV).

In some embodiments, the pharmaceutical composition (e.g., diluted pharmaceutical composition) or the diluted reference pharmaceutical composition comprises at least 2 times more, at least 3 times more, at least 4 times more, at least 5 times more, at least 6 times more, at least 7 times more, at least 8 times more, at least 9 times more, at least 10 times more, at least 15 times more, at least 20 times more, at least 50 times more, at least 100 times more, at least 5% more, at least 10% more, at least 15% more, at least 20% more, at least 25% more, at least 30% more, at least 35% more, at least 40%, at least 45% more, at least 50% more, at least 55% more, at least 60% more, at least 65% more, at least 70% more, at least 75% more, at least 80% more, at least 85% more, at least 90% more, at least 95% more, at least 100% more, at least 150% more, or at least 200% more, at least 250% more, or at least 300%, at least 400% more, or at least 500% more aggregated viral vectors (e.g., aggregated AAV) as compared to a control solution or to the pharmaceutical composition prior to dilution or to the reference pharmaceutical composition prior to dilution.

In some embodiments, a molecular diameter of viral vectors (e.g., level of viral vector aggregation) is determined by any suitable method or any suitable assay (see Section 4.6). In some embodiments, a molecular diameter of viral vectors is measured to determine the amount of viral vector aggregation (e.g., AAV aggregation). In some embodiments, the molecular diameter of the viral vectors in the pharmaceutical composition (e.g., diluted pharmaceutical composition) or in the diluted reference pharmaceutical composition higher than the molecular diameter of the viral vectors in the pharmaceutical composition prior to dilution, or in a control solution, or in the reference pharmaceutical composition prior to dilution. In some embodiments, the pharmaceutical composition (e.g., diluted pharmaceutical composition) comprises viral vectors that have (e.g., in average) a diameter that is at least 2 times higher, at least 3 times higher, at least 4 times higher, at least 5 times higher, at least 6 times higher, at least 7 times higher, at least 8 times higher, at least 9 times higher, at least 10 times higher, at least 15 times higher, at least 20 times higher, at least 50 times higher, at least 100 times higher, at least 5% higher, at least 10% higher, at least 15% higher, at least 20% higher, at least 25% higher, at least 30% higher, at least 35% higher, at least 40%, at least 45% higher, at least 50% higher, at least 55% higher, at least 60% higher, at least 65% higher, at least 70% higher, at least 75% higher, at least 80% higher, at least 85% higher, at least 90% higher, at least 95% higher, at least 100% higher, at least 150% higher, or at least 200% higher, at least 250% higher, or at least 300%, at least 400% higher, or at least 500% higher than the diameter of viral vectors in a control solution or in the pharmaceutical composition prior to dilution or in the reference pharmaceutical composition. In some embodiments, the pharmaceutical composition (e.g., diluted pharmaceutical composition) or the diluted reference pharmaceutical composition comprises viral vectors that have (e.g., in average) diameter that is about or at least about 10 nm, 15 nm, 20 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 45 nm, 50 nm, 55 nm, 60 nm, 65 nm, 70 nm, 75 nm, 80 nm, 85 nm, 90 nm, 95 nm, 100 nm, 125 nm, 150 nm, 175 nm, 200 nm, 225 nm, 250 nm, 275 nm, 300 nm, 325 nm, 350 nm, 375 nm, 400 nm, 450 nm, 500 nm, or over 500 nm. In some embodiments, the pharmaceutical composition prior to dilution, or the reference pharmaceutical composition prior to dilution, or a control comprises viral vectors that have (e.g., in average) diameter that is about or at most about 5 nm, 10 nm, 15 nm, 20 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 45 nm, 50 nm, 55 nm, 60 nm, 65 nm, 70 nm, 75 nm, 80 nm, 85 nm, 90 nm, 95 nm, or 100 nm. In some embodiments, the pharmaceutical composition prior to dilution, or the reference pharmaceutical composition prior to dilution, or a control comprises viral vectors that have (e.g., in average) diameter that is about or at most 25 nm, 30 nm, 35 nm, or 40 nm.

In some embodiments, molecular diameter or level of viral vector aggregation is measured 30 minutes, 1 hr, 2 hrs, 3 hrs, 4 hrs, 5 hrs, 6 hrs, 7 hrs, 8 hrs, 9 hrs, 10 hrs, 15 hrs, 20 hrs, 24 hrs, one day, 2 days, 3 days, 4 days, 5 days, 6 days, 7 days, two weeks, three weeks, four weeks, 2 months, 3 months, 4 months, 5 months 6 months, 1 year, 2 years 5 years, or more than 5 years after the pharmaceutical composition is diluted. In some embodiments, molecular diameter or level of viral vector aggregation is measured prior to administration (e.g., suprachoroidal administration). In some embodiments, molecular diameter or level of viral vector aggregation is measured right before administration (e.g., suprachoroidal administration). In some embodiments, molecular diameter or level of viral vector aggregation is measured 30 minutes, 1 hr, 2 hrs, 3 hrs, 4 hrs, 5 hrs, 6 hrs, 7 hrs, 8 hrs, 9 hrs, 10 hrs, 15 hrs, 20 hrs, 24 hrs, or 2 days before administration of the pharmaceutical composition (e.g., diluted pharmaceutical composition), the diluted reference pharmaceutical composition, the undiluted pharmaceutical composition, undiluted reference pharmaceutical composition, or of a control (e.g., suprachoroidal administration). In some embodiments, molecular diameter or level of viral vector aggregation is measured after long-term storage (e.g., storage at 25° C., 20° C., 4° C., −80° C.). In some embodiments, molecular diameter or level of viral vector aggregation is measured after a flash frozen diluted pharmaceutical composition is thawed.

In some embodiments, the diluted pharmaceutical composition, the undiluted pharmaceutical composition, a reference pharmaceutical composition, or a control have the same vector genome concentration. In some embodiments, the diluted pharmaceutical composition, the undiluted pharmaceutical composition, a reference pharmaceutical composition, or a control have the same amount of genome copies. In some embodiments, the pharmaceutical composition (e.g., diluted pharmaceutical composition) has at least the same vector genome concentration as the undiluted pharmaceutical composition (or as a control or a reference pharmaceutical composition). In some embodiments, the pharmaceutical composition (e.g., diluted pharmaceutical composition) has at least the same amount of genome copies as the undiluted pharmaceutical composition (or as a control or a reference pharmaceutical composition). In some embodiments, the pharmaceutical composition (e.g., diluted pharmaceutical composition) has at least the same viral vector potency (e.g., AAV in vitro potency) as the undiluted pharmaceutical composition (or as a control or a reference pharmaceutical composition).

In some embodiments, the ionic strength of the pharmaceutical composition (e.g., diluted pharmaceutical composition) is increased after administration (e.g., suprachoroidal administration). In some embodiments, the level of viral vector aggregation (clustering) is reduced after administration. In some embodiments, viral vector aggregation (clustering) in a pharmaceutical composition prior to administration allows for the viral vector or active ingredient to remain at the site of injection longer as compared to a solution comprising the viral vector but with no viral vector aggregation or as compared to a solution comprising the viral vector having a reduced amount of viral vector aggregation. In some embodiments, viral vector aggregation (clustering) in a pharmaceutical composition prior to administration results in increased thickness at the site of injection after administration as compared to the thickness at the site of injection obtained after a solution comprising the viral vector but with no viral vector aggregation or after a solution comprising the viral vector having a reduced amount of viral vector aggregation is administered. In some embodiments, viral vector aggregation (clustering) in a pharmaceutical composition prior to administration results in a smaller circumferential spread at the site of injection after administration as compared to the circumferential spread at the site of injection obtained after a solution comprising the viral vector but with no viral vector aggregation or after a solution comprising the viral vector having a reduced amount of viral vector aggregation is administered. In some embodiments, viral vector aggregation (clustering) in a pharmaceutical composition prior to administration results in a higher clearance time at the site of injection after administration as compared to the clearance time at the site of injection obtained after a solution comprising the viral vector but with no viral vector aggregation or after a solution comprising the viral vector having a reduced amount of viral vector aggregation is administered. In some embodiments, viral vector aggregation (clustering) in a pharmaceutical composition prior to administration results in a reduced level of vasodilation and/or vascular leakage after administration as compared to the level of vasodilation and/or vascular leakage obtained after a solution comprising the viral vector but with no viral vector aggregation or after a solution comprising the viral vector having a reduced amount of viral vector aggregation is administered. In some embodiments, viral vector aggregation (clustering) in a pharmaceutical composition prior to administration results in a higher rate of transduction (rate of infection) at the site of injection after administration as compared to the rate of transduction (rate of infection) at the site of injection obtained after a solution comprising the viral vector but with no viral vector aggregation or after a solution comprising the viral vector having a reduced amount of viral vector aggregation is administered. In some embodiments, viral vector aggregation (clustering) in a pharmaceutical composition prior to administration results in a higher AAV levels at the site of injection after administration as compared to the AAV levels at the site of injection obtained after a solution comprising the viral vector but with no viral vector aggregation or after a solution comprising the viral vector having a reduced amount of viral vector aggregation is administered. In some embodiments, viral vector aggregation (clustering) in a pharmaceutical composition prior to administration results in a higher transgene expression levels in the eye after administration as compared to the transgene expression levels in the eye obtained after a solution comprising the viral vector but with no viral vector aggregation or after a solution comprising the viral vector having a reduced amount of viral vector aggregation is administered. In some embodiments, a diluted pharmaceutical composition refers to a pharmaceutical composition having a lower ionic strength and/or a lower salt concentration as compared to a reference pharmaceutical composition. In some embodiments, a diluted pharmaceutical composition refers to a pharmaceutical composition comprising an ionic strength of at most about 200 mM. In some embodiments, a diluted pharmaceutical composition refers to a pharmaceutical composition comprising at least about 3% viral vector aggregation (e.g., AAV aggregation).

In some embodiments, the pharmaceutical composition (e.g., diluted pharmaceutical composition) has an amount of viral vector aggregation sufficient to expand at least a portion of the site of injection (e.g. SCS) to a thickness of at least 500 μm or about 500 μm to about 3 mm, for at least two hours after administration. In some embodiments, the amount of viral vector aggregation (e.g., AAV aggregation) of the pharmaceutical composition (e.g., diluted pharmaceutical composition) is sufficient to expand the site of injection (e.g. SCS) to a thickness of about 750 μm to about 2.8 mm, about 750 μm to about 2.5 mm, about 750 μm to about 2 mm, or about 1 mm to about 2 mm. In some embodiments, the amount of viral vector aggregation (e.g., AAV aggregation) in the pharmaceutical composition (e.g., diluted pharmaceutical composition) is sufficient to expand the site of injection (e.g. SCS) to a thickness of about 500 μm to about 3.0 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the amount of viral vector aggregation (e.g., AAV aggregation) in the pharmaceutical composition (e.g., diluted pharmaceutical composition) is sufficient to expand the site of injection (e.g. SCS) to a thickness of about 1 mm to about 3 mm for at least two hours, at least three hours, at least four hours, at least five hours, at least six hours, at least seven hours, at least eight hours, at least ten hours, at least twelve hours, at least eighteen hours, or at least twenty-four hours after administration. In some embodiments, the amount of viral vector aggregation (e.g., AAV aggregation) in the pharmaceutical composition (e.g., diluted pharmaceutical composition) is sufficient to expand the site of injection (e.g. SCS) to a thickness of about 1 mm to about 2 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the amount of viral vector aggregation (e.g., AAV aggregation) in the pharmaceutical composition (e.g., diluted pharmaceutical composition) is sufficient to expand the site of injection (e.g. SCS) to a thickness of about 2 mm to about 3 mm for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years after the administration. In some embodiments, the amount of viral vector aggregation (e.g., AAV aggregation) in the pharmaceutical composition (e.g., diluted pharmaceutical composition) is sufficient to expand the site of injection (e.g. SCS) to a thickness of about 750 μm to about 2.8 mm, about 750 μm to about 2.5 mm, about 750 μm to about 2 mm, or about 1 mm to about 2 mm for an indefinite period. An indefinite period may be achieved due, at least in part, to the stability of the pharmaceutical composition (e.g., diluted pharmaceutical composition) in the site of injection (e.g. SCS).

In some embodiments, a pharmaceutical composition (e.g., diluted pharmaceutical composition) has an amount of viral vector aggregation (e.g., AAV aggregation) sufficient to expand the site of injection (e.g. SCS) to a thickness of at least 500 μm, or about 500 μm to about 3 mm. In some embodiments, a pharmaceutical composition (e.g., diluted pharmaceutical composition) has an amount of viral vector aggregation (e.g., AAV aggregation) sufficient to expand the site of injection (e.g. SCS) to a thickness of at least about 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger than 10 mm. In some embodiments, a reference pharmaceutical composition or an undiluted pharmaceutical composition is capable to expand the site of injection to a thickness of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

Also provided herein are methods of treating a disease (e.g., an ocular disease) described in Section 4.5 using the pharmaceutical compositions (e.g., diluted pharmaceutical composition) disclosed herein. In some embodiments, a method of treating an ocular disease includes administering an effective amount of the pharmaceutical composition (e.g., recombinant adeno-associated virus (AAV) vector comprising an expression cassette encoding a transgene) to a subject (e.g., human). In some embodiments, the pharmaceutical composition is administered in the suprachoroidal space (SCS) of an eye of the subject. In some embodiments, the effective amount of the pharmaceutical composition sufficient to elicit a therapeutic response when administered to the SCS is less than the effective amount of the pharmaceutical composition sufficient to elicit a therapeutic response when administered subretinally. In some embodiments, the effective amount of the pharmaceutical composition sufficient to elicit a therapeutic response when administered to the SCS is less than the effective amount of the pharmaceutical composition sufficient to elicit a therapeutic response when administered intravitreously. In some embodiments, the pharmaceutical composition has the same vector genome concentration when administered to the SCS as when administered via subretinal administration or via intravitreous administration. In some embodiments, the pharmaceutical composition has the same amount of genome copies when administered to the SCS as when administered via subretinal administration or via intravitreous administration. In some embodiments, the effective amount of the pharmaceutical composition sufficient to elicit a therapeutic response in a subject is lower as compared to the effective amount of a reference pharmaceutical composition sufficient to elicit a therapeutic response in the subject when administered to the SCS. In some embodiments, a reference pharmaceutical composition is a pharmaceutical composition before it is diluted. In some embodiments, a reference pharmaceutical composition has higher ionic strength as compared to the pharmaceutical composition. In some embodiments, the pharmaceutical composition has a higher level of viral vector aggregation (e.g., AAV aggregation) as compared to a reference pharmaceutical composition. In some embodiments, the effective amount of the pharmaceutical composition sufficient to elicit a therapeutic response when administered to the SCS is less than the effective amount of a reference pharmaceutical composition sufficient to elicit a therapeutic response when administered subretinally. In some embodiments, the effective amount of the pharmaceutical composition sufficient to elicit a therapeutic response when administered to the SCS is less than the effective amount of a reference pharmaceutical composition sufficient to elicit a therapeutic response when administered intravitreously. In some embodiments, the pharmaceutical composition and the reference pharmaceutical composition have the same vector genome concentration. In some embodiments, the pharmaceutical composition and the reference pharmaceutical composition have the same amount of genome copies.

Also provided herein are methods of preparing a pharmaceutical composition. In some embodiments, a method of preparing a pharmaceutical composition includes preparing a composition (e.g., a reference pharmaceutical composition) comprising phosphate-buffered saline, sucrose, and a therapeutically effective amount of a recombinant adeno-associated virus (AAV) vector comprising an expression cassette encoding a transgene. In some embodiments, a method of preparing a pharmaceutical composition includes admixing a solution comprising phosphate-buffered saline and sucrose to a composition comprising AAV. In some embodiments, the pharmaceutical composition has lower ionic strength and/or a higher level of aggregated recombinant AAV than the composition (or reference pharmaceutical composition). In some embodiments, the pharmaceutical composition has an ionic strength of about or of at most about 135 mM. In some embodiments, the pharmaceutical composition has an ionic strength of about or of at most about 40 mM. In some embodiments, the pharmaceutical composition has an ionic strength of about or of at most about 20 mM. In some embodiments, the pharmaceutical composition has substantially the same tonicity or osmolality as the composition.

Provided herein are methods of preparing a pharmaceutical composition including admixing a solution comprising phosphate-buffered saline and sucrose to a composition (e.g., a composition having a recombinant adeno-associated virus (AAV) vector comprising an expression cassette encoding a transgene). In some embodiments, the pharmaceutical composition has lower ionic strength and/or a higher level of aggregated recombinant AAV than the composition. In some embodiments, the pharmaceutical composition has an ionic strength of about or of at most about 135 mM. In some embodiments, the pharmaceutical composition has an ionic strength of about or of at most about 40 mM. In some embodiments, the pharmaceutical composition has an ionic strength of about or of at most about 20 mM. In some embodiments, the pharmaceutical composition has substantially the same tonicity or osmolality as the composition.

Also provided herein are kits for preparing a pharmaceutical composition. In some embodiments, a kit includes (i) a composition comprising a recombinant adeno-associated virus (AAV) vector comprising an expression cassette encoding a transgene; and (ii) a solution comprising phosphate-buffered saline and sucrose. In some embodiments, a kit can include instructions for admixing the composition with the solution. In some embodiments, the instructions includes instructions on admixing the solution with the composition to obtain a pharmaceutical composition. In some embodiments, the composition comprises a phosphate-buffered saline and sucrose. In some embodiments, the composition comprises 4% sucrose. In some embodiments, the solution comprises 10% sucrose. In some embodiments, the pharmaceutical composition has an ionic strength of about or of at most about 135 mM. In some embodiments, the pharmaceutical composition has an ionic strength of about or of at most about 40 mM. In some embodiments, the pharmaceutical composition has an ionic strength of about or of at most about 20 mM. In some embodiments, the pharmaceutical composition has substantially the same tonicity or osmolality as the composition.

In some embodiments, any of the methods, or the pharmaceutical composition, or the kits of the present disclosure result in at least some of the aggregated recombinant AAV in the pharmaceutical composition to disaggregate after the pharmaceutical composition is administered to the suprachoroidal space of an eye of a human subject. In some embodiments, aggregation of the recombinant AAV is capable of being reversed upon suprachoroidal administration of the pharmaceutical composition to a human subject. In some embodiments, the aggregated recombinant AAVs turn to monomers or become less aggregated once injected in the SCS of a subject. In some embodiments, the composition comprises potassium chloride, potassium phosphate monobasic, sodium chloride, sodium phosphate dibasic anhydrous, sucrose, and optionally a surfactant. In some embodiments, the composition comprises modified Dulbecco's phosphate-buffered saline solution, and optionally a surfactant. In some embodiments, the composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and a surfactant. In some embodiments, the solution comprises a phosphate-buffered sodium chloride and sucrose. In some embodiments, admixing the solution with the composition dilutes the composition by about or at least about 2-fold, 3-fold, 4-fold, 5-fold, 6-fold, 7-fold, 8-fold, 9-fold or 10-fold. In some embodiments, admixing the solution with the composition occurs on the same day that the pharmaceutical composition is administered to the suprachoroidal space of an eye of a human subject. In some embodiments, admixing the solution with the composition occurs within 24 hours of the pharmaceutical composition being administered to the suprachoroidal space of an eye of a human subject. In some embodiments, the pharmaceutical composition includes about 1.0×10¹² to about 3.0×10¹² genome copies of the recombinant AAV. In some embodiments, the recombinant AAV includes components from AAV8 and the pharmaceutical composition has an ionic strength between about 30 mM to about 60 mM. In some embodiments, the recombinant AAV includes components from AAV9 and the pharmaceutical composition has an ionic strength between about 15 mM to about 30 mM. In some embodiments, the recombinant AAV includes components from AAV2 and the pharmaceutical composition has an ionic strength between about 100 mM to about 200 mM.

In some embodiments, the pharmaceutical composition is substantially localized near the insertion site (see Section 4.2.1 and Section 4.2.2). In some embodiments, the pharmaceutical composition results in a higher level of transgene expression (concentration) when the pharmaceutical composition is administered in the SCS as compared to when the pharmaceutical composition is administered subretinally or intravitreously (see Section 4.2.5). In some embodiments, the pharmaceutical composition results in a higher level of transgene expression (concentration) when the pharmaceutical composition is administered in the SCS as compared to when a reference pharmaceutical composition is administered subretinally, intravitreously, or in the SCS (see Section 4.2.5). In some embodiments, the pharmaceutical composition results in a higher level of AAV when the pharmaceutical composition is administered in the SCS as compared to when the pharmaceutical composition is administered subretinally or intravitreously (see Section 4.2.4). In some embodiments, the pharmaceutical composition results in a higher level of AAV when the pharmaceutical composition is administered in the SCS as compared to when a reference pharmaceutical composition is administered subretinally, intravitreously, or in the SCS (see Section 4.2.4). In some embodiments, the pharmaceutical composition results in a higher rate of transduction (rate of infection) at a site of injection when the pharmaceutical composition is administered in the SCS as compared to when the pharmaceutical composition is administered subretinally or intravitreously (see Section 4.2.4). In some embodiments, the pharmaceutical composition results in a higher rate of transduction (rate of infection) at a site of injection when the pharmaceutical composition is administered in the SCS as compared to when a reference pharmaceutical composition is administered subretinally, intravitreously, or in the SCS (see Section 4.2.4). In some embodiments, the pharmaceutical composition results in reduced vasodilation and/or vascular leakage when the pharmaceutical composition is administered in the SCS as compared to when the pharmaceutical composition is administered subretinally or intravitreously. In some embodiments, the pharmaceutical composition results in reduced vasodilation and/or vascular leakage when the pharmaceutical composition is administered in the SCS as compared to when a reference pharmaceutical composition is administered subretinally, intravitreously, or in the SCS. In some embodiments, the reference pharmaceutical composition includes the recombinant adeno-associated virus (AAV) vector comprising the expression cassette encoding the transgene. In some embodiments, the pharmaceutical composition has a higher level of AAV aggregation than the reference pharmaceutical composition. In some embodiments, the pharmaceutical composition and the reference pharmaceutical composition have the same vector genome concentration. In some embodiments, the pharmaceutical composition and the reference pharmaceutical composition have the same amount of genome copies.

### 4.1.1 Dilution of Ionic Strength

In some embodiments, a pharmaceutical composition is diluted with a solution containing a lower ionic strength to reduce the ionic strength of the pharmaceutical composition. In some embodiments, the pharmaceutical composition is diluted prior to administration. In some embodiments, the pharmaceutical composition is diluted and stored. In some embodiments, a solution containing lower ionic strength as compared to the pharmaceutical composition is added to the pharmaceutical composition to provide pharmaceutical compositions comprising 5%, 10%, 15%, 20%, 25%, 30%, 50%, 55%, 60%, 65%, 70%, 75%, 80%, 85%, 90%, or 95% the ionic strength of the undiluted pharmaceutical composition (e.g., diluted two-times, three-times, four-times, eight-times, or ten-times). In some embodiments, the pharmaceutical composition is diluted with a phosphate-buffered 10% sucrose solution. In some embodiments, the pharmaceutical composition comprises modified DPBS with 4% sucrose. In some embodiments, the pharmaceutical composition comprises a poloxamer (e.g., P188).

In some embodiments, the solutions containing lower ionic strength and that are used to dilute the pharmaceutical composition comprises a lower amount or concentration of a salt as compared to the undiluted pharmaceutical composition. Examples of salts include, but are not limited to, sodium chloride, sodium sulfate, and ammonium sulfate.

In some embodiments, the solutions containing lower ionic strength and used for dilutions comprise about or at most about 1%, 5%, 10%, 15%, 20%, 25%, 30%, 50%, 55%, 60%, 65%, 70%, 75%, 80%, 85%, 90%, or 95% of the salt concentration in the undiluted pharmaceutical composition. In some embodiments, the solutions containing lower ionic strength and used for dilutions does not comprise a salt. In some embodiments, the solution (e.g., solutions containing lower ionic strength) used for dilutions comprise about or at most about 0 mM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM 75 mM, 80 mM, 85 mM, 90 mM, 95 mM, 100 mM, 110 mM, 115 mM, 120 mM, 125 mM, 130 mM, 135 mM, 140 mM, 145 mM, 150 mM, 155 mM, 160 mM, 165 mM, 170 mM 175 mM, 180 mM, 185 mM, 190 mM, 195 mM, 200 mM, 250 mM, 300 mM, 350 mM, 400 mM, 450 mM, 500 mM, 550 mM, or 600 mM of a salt (e.g., NaCl). In some embodiments, the solution (e.g., solutions containing lower ionic strength) used for dilutions does not comprise salt. In some embodiments, the solution (e.g., solutions containing lower ionic strength) used for dilutions has a salt concentration of about 0 mM to about 30 mM, 0 mM to about 25 mM, 0 mM to about 100 mM, 0 mM to about 50 mM, 0 mM to about 200 mM, 5 mM to about 100 mM, 10 mM to about 30 mM, 10 mM to about 40 mM, 10 mM to about 50 mM, 10 mM to about 60 mM, 10 mM to about 100 mM, 5 mM to about 50 mM, 5 mM to about 30 mM, 1 mM to about 100 mM, 1 mM to about 40 mM, or 1 mM to about 30 mM, 1 mM to about 200 mM, 1 mM to about 600 mM, or 1 mM to about 300 mM. In some embodiments, the solution (e.g., solutions containing lower ionic strength) used for dilutions comprise about or at most about 10 mM of salt. In some embodiments, the solution (e.g., solutions containing lower ionic strength) used for dilutions comprise about or at most about 100 mM of salt. In some embodiments, the solution (e.g., solutions containing lower ionic strength) used for dilutions comprise about or at most about 200 mM of salt. In some embodiments, the solutions containing lower ionic strength and used for dilutions comprises sucrose. In some embodiments, the solutions containing lower ionic strength and used for dilutions comprises 4%, 6%, 8%, 10%, 15%, 20%, 25%, 30% (or higher) sucrose. In some embodiments, the solution (e.g., solutions containing lower ionic strength) used for dilutions comprises 4% sucrose. In some embodiments, the solution used for dilutions comprises 6% sucrose. In some embodiments, the solution used for dilutions comprises 10% sucrose. In some embodiments, the pharmaceutical composition comprises about, at least about, or at most about: 0.5% (w/v), 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, or higher than 40% sucrose.

In some embodiments, the solution (e.g., solutions containing lower ionic strength) used for dilutions has an ionic strength of about or at most about 0 mM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM 75 mM, 80 mM, 85 mM, 90 mM, 95 mM, 100 mM, 110 mM, 115 mM, 120 mM, 125 mM, 130 mM, 135 mM, 140 mM, 145 mM, 150 mM, 155 mM, 160 mM, 165 mM, 170 mM 175 mM, 180 mM, 185 mM, 190 mM, 195 mM, 200 mM, 250 mM, 300 mM, 350 mM, 400 mM, 450 mM, 500 mM, 550 mM, or 600 mM. In some embodiments, the solution (e.g., solutions containing lower ionic strength) used for dilutions has an ionic strength of about or at most about 25 mM. In some embodiments, the solution (e.g., solutions containing lower ionic strength) used for dilutions has an ionic strength of about or at most about 50 mM. In some embodiments, the solution (e.g., solutions containing lower ionic strength) used for dilutions has an ionic strength of about or at most about 15 mM. In some embodiments, the solution (e.g., solutions containing lower ionic strength) used for dilutions has an ionic strength of about or at most about 80 mM. In some embodiments, the solution (e.g., solutions containing lower ionic strength) used for dilutions has an ionic strength of about or at most about 100 mM. In some embodiments, the solution (e.g., solutions containing lower ionic strength) used for dilutions has an ionic strength of about 5 mM to about 100 mM, 10 mM to about 30 mM, 10 mM to about 40 mM, 10 mM to about 50 mM, 10 mM to about 60 mM, 10 mM to about 100 mM, 5 mM to about 50 mM, 5 mM to about 30 mM, 1 mM to about 100 mM, 1 mM to about 40 mM, or 1 mM to about 30 mM, 1 mM to about 200 mM, 1 mM to about 600 mM, or 1 mM to about 300 mM.

In some embodiments, a low ionic strength pharmaceutical composition is used to administer an AAV encoding a transgene. In some embodiments, a pharmaceutical composition (e.g., diluted liquid formulation) having a reduced ionic strength as compared to a reference is used to administer an AAV encoding a transgene. In some embodiments, a low salt pharmaceutical composition (e.g., diluted liquid formulation) is used to administer an AAV encoding a transgene. In some embodiments, a pharmaceutical composition having a lower salt concentration as compared to a control solution, or as compared to a reference pharmaceutical composition, or as compared to PBS, or as compared to a commonly used pharmaceutical composition for subretinal injection, is used to administer an AAV encoding a transgene.

In some embodiments, examples of compounds that can be used to prepare a salt includes (but not limited to) aluminum, acetate, glutamate, mucate, arginine, aspartate, glycolate, napsylate, benzathine, benzenesulfonate, glycollylarsanilate, nitrate, calcium, benzoate, hexanoate, octanoate, chloroprocaine, besylate, hexylresorcinate, oleate, choline, bicarbonate, hydrabamine, pamoate, diethanolamine, bitartrate, hydroxynaphthoate, pantothenate, ethanolamine, bromide, iodide, phosphate, ethylenediamine, camsylate, isethionate, polygalacturonate, histidine, carbonate, isethionate, propionate, lithium, chloride, lactate, salicylate, lysine, citrate, lactobionate, stearate, magnesium, decanoate, malate, subacetate, meglumine, edetate, maleate, succinate, potassium, estolate, mandelate, sulfate, procaine, esylate, mesylate, tartrate, sodium, fumarate, methylbromide, teoclate, trimethylamine, gluceptate, methylnitrate, tosylate, zinc, gluconate, methylsulfate, and triethiodide.

In some embodiments, a salt in a solution or to be used in a pharmaceutical composition includes, but it is not limited to, sodium fluoride, sodium chloride, sodium bromide, sodium iodide, sodium sulfate, sodium bicarbonate, sodium carbonate, sodium amide (NaNH₂), or any salt suitable or pharmaceutical formulation.

### 4.1.2 Other Components of the Formulation

In some embodiments, the disclosure provides a pharmaceutical composition (e.g., diluted formulation or lower ionic strength formulation) comprising a recombinant adeno-associated virus (AAV) and at least one of potassium phosphate monobasic, sodium chloride, sodium phosphate dibasic anhydrous, sucrose, and surfactant. In some embodiments, the pharmaceutical composition (e.g., diluted formulation or lower ionic strength formulation) does not comprise sucrose.

In some embodiments, the disclosure provides a pharmaceutical composition comprising a recombinant adeno-associated virus (AAV) and at least one of an ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, and calcium citrate. In some embodiments, the surfactant can be one or more components from the group consisting of poloxamer 188, polysorbate 20, and polysorbate 80.

In some embodiments, the pharmaceutical composition comprises about, at least about, or at most about: 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, 1 mg/mL, 1.05 mg/mL, 1.10 mg/mL, 1.15 mg/mL, 1.20 mg/mL, 1.25 mg/mL, 1.30 mg/mL, 1.35 mg/mL, 1.40 mg/mL, 1.45 mg/mL, 1.50 mg/mL, or more than 1.50 mg/mL of sodium phosphate dibasic anhydrous (or an equivalent). In some embodiments, the pharmaceutical composition comprises about, at least about, or at most about: 4.5 mg/mL, 4.55 mg/mL, 4.6 mg/mL, 4.65 mg/mL, 4.7 mg/mL, 4.75 mg/mL, 4.8 mg/mL, 4.85 mg/mL, 4.9 mg/mL, 4.95 mg/mL, 5 mg/mL, 5.05 mg/mL, 5.10 mg/mL, 5.15 mg/mL, 5.20 mg/mL, 5.25 mg/mL, 5.30 mg/mL, 5.35 mg/mL, 5.40 mg/mL, 5.45 mg/mL, 5.50 mg/mL, 5.55 mg/mL, 5.60 mg/mL, 5.65 mg/mL, 5.70 mg/mL, 5.75 mg/mL, 5.80 mg/mL, 5.81 mg/mL, 5.82 mg/mL, 5.83 mg/mL, 5.84 mg/mL, 5.85 mg/mL, 5.86 mg/mL, 5.87 mg/mL, 5.88 mg/mL, 5.89 mg/mL, 5.90 mg/mL, 5.95 mg/mL, 6 mg/mL, or more than 6 mg/mL of sodium chloride (or an equivalent). In some embodiments, the pharmaceutical composition comprises about, at least about, or at most about: 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, or more than 1 mg/mL of potassium chloride and/or potassium phosphate monobasic (or equivalents thereof).

In certain embodiments, the pharmaceutical composition has an ionic strength of about 60 mM to about 115 mM. In certain embodiments, the pharmaceutical composition has an ionic strength of about 60 mM to about 100 mM. In certain embodiments, the pharmaceutical composition has an ionic strength of about 65 mM to about 95 mM. In certain embodiments, the pharmaceutical composition has an ionic strength of about 70 mM to about 90 mM. In certain embodiments, the pharmaceutical composition has an ionic strength of about 75 mM to about 85 mM.

In certain embodiments, the pharmaceutical composition has an ionic strength of about 30 mM to about 100 mM. In certain embodiments, the pharmaceutical composition has an ionic strength of about 35 mM to about 95 mM. In certain embodiments, the pharmaceutical composition has an ionic strength of about 40 mM to about 90 mM. In certain embodiments, the pharmaceutical composition has an ionic strength of about 45 mM to about 85 mM. In certain embodiments, the pharmaceutical composition has an ionic strength of about 50 mM to about 80 mM. In certain embodiments, the pharmaceutical composition has an ionic strength of about 55 mM to about 75 mM. In certain embodiments, the pharmaceutical composition has an ionic strength of about 60 mM to about 70 mM.

In certain embodiments, the pharmaceutical composition comprises potassium chloride (e.g., at a concentration of 0.2 g/L). In certain embodiments, the pharmaceutical composition comprises potassium phosphate monobasic (e.g., at a concentration of 0.2 g/L). In certain embodiments, the pharmaceutical composition comprises sodium chloride (e.g., at a concentration of 5.84 g/L). In certain embodiments, the pharmaceutical composition comprises sodium phosphate dibasic anhydrous (e.g., at a concentration of 1.15 g/L). In certain embodiments, the pharmaceutical composition comprises potassium chloride, potassium phosphate monobasic, sodium chloride, and sodium phosphate dibasic anhydrous.

In some embodiments, the reference pharmaceutical composition comprises the same components as the pharmaceutical composition. In some embodiments, the reference pharmaceutical composition comprises the same components as the pharmaceutical composition but has lower ionic strength than the pharmaceutical composition. In some embodiments, the reference pharmaceutical composition comprises the same components as the pharmaceutical composition with the exception of one or more components that affect or increase ionic strength of a composition or solution.

In certain embodiments, the pharmaceutical composition comprises sucrose at a concentration of 10% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L). In certain embodiments, the pharmaceutical composition comprises polysorbate 20 at a concentration of 0.0005% (weight/volume, 0.05 g/L) to 0.05% (weight/volume, 0.5 g/L). In certain embodiments, the pharmaceutical composition comprises polysorbate 80 at a concentration of 0.0005% (weight/volume, 0.05 g/L) to 0.05% (weight/volume, 0.5 g/L). In some embodiments, the pharmaceutical composition comprises a surfactant (e.g., poloxamer 188, polysorbate 20, and/or polysorbate 80) at a concentration of about, at least about, or at most about: 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 0.1%, or more than 0.1%.

In certain embodiments, the pH of the pharmaceutical composition is about 7.4. In certain embodiments, the pH of the pharmaceutical composition is about 6.0 to 9.0. In certain embodiments, the pH of the pharmaceutical composition is 7.4. In certain embodiments, the pH of the pharmaceutical composition is 6.0 to 9.0.

In certain embodiments, the pharmaceutical composition is in a hydrophobically-coated glass vial. In certain embodiments, the pharmaceutical composition is in a Cyclo Olefin Polymer (COP) vial. In certain embodiments, the pharmaceutical composition is in a Daikyo Crystal Zenith® (CZ) vial. In certain embodiments, the pharmaceutical composition is in a TopLyo coated vial.

In certain embodiments, disclosed herein is a pharmaceutical composition comprising a recombinant AAV and at least one of: (a) potassium chloride at a concentration of 0.2 g/L, (b) potassium phosphate monobasic at a concentration of 0.2 g/L, (c) sodium chloride at a concentration of 5.84 g/L, (d) sodium phosphate dibasic anhydrous at a concentration of 1.15 g/L, (e) sucrose at a concentration of 4% weight/volume (40 g/L), (f) poloxamer 188 at a concentration of 0.001% weight/volume (0.01 g/L), and (g) water, and wherein the recombinant AAV is AAV8. In some embodiments, the pharmaceutical composition does not comprise sucrose. In certain embodiments, disclosed herein is a pharmaceutical composition comprising a composition comprising recombinant AAV and a solution comprising 10% Sucrose, 2.70 mM potassium chloride, 8.10 mM sodium phosphate dibasic anhydrous, 1.47 mM potassium phosphate monobasic, 292 mM sucrose, 0.001% (0.01 mg/mL) poloxamer 188, pH 7.4, wherein, for example, the composition and solution are admixed at a composition to solution ratio of 1 to 9. In some embodiments, disclosed herein is a method for preparing a pharmaceutical composition by admixing the composition and solution at a composition to solution ratio of 1 to 9. In some embodiments, disclosed herein is a kit used for preparing a pharmaceutical composition by admixing the composition and solution at a composition to solution ratio of 1 to 9. In some embodiments, the composition and solution are admixed at a composition to solution ratio of 1 to 1, 1 to 2, 1 to 3, 1 to 4, 1 to 5, 1 to 6, 1 to 7, 1 to 8, 1 to 9, 1 to 10, 1 to 12, 1 to 15, 1 to 17, 1 to 20, 1 to 25, 1 to 30, 1 to 35, 1 to 40, or more than 1 to 40.

In some embodiments, the pharmaceutical composition comprises (a) an AAV8 or AAV9 that encodes Tripeptidyl-Peptidase 1 and at least one of: (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water. In some embodiments, the pharmaceutical composition does not comprise sucrose. In some embodiments, the level of AAV aggregation in the pharmaceutical composition impacts Batten-CLN2-associated vision loss.

In some embodiments, the pharmaceutical composition has desired viscosity, density, and/or osmolality that is suitable for suprachoroidal injection (for example, via a suprachoroidal drug delivery device such as a microinjector with a microneedle). In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%. In some embodiments, the pharmaceutical composition is a lyophilized composition comprising a residual moisture content between about 2% and about 6%. In some embodiments, the pharmaceutical composition is a lyophilized composition comprising a residual moisture content between about 10% and about 4%. In some embodiments, the pharmaceutical composition is a lyophilized composition comprising a residual moisture content of about 5%.

In certain embodiments, the pharmaceutical composition has a osmolality range of 200 mOsm/L to 660 mOsm/L. In certain embodiments, the pharmaceutical composition has a osmolality of about, of at least about, or of at most about: 200 mOsm/L, 250 mOsm/L, 300 mOsm/L, 350 mOsm/L, 400 mOsm/L, 450 mOsm/L, 500 mOsm/L, 550 mOsm/L, 600 mOsm/L, 650 mOsm/L, or 660 mOsm/L.

In certain embodiments, gene therapy constructs are supplied as a frozen sterile, single use solution of the AAV vector active ingredient in a formulation buffer. In a specific embodiment, the pharmaceutical compositions suitable for suprachoroidal administration comprise a suspension of the recombinant vector in a formulation buffer comprising a physiologically compatible aqueous buffer, a surfactant and optional excipients. In some embodiments, the construct is formulated in Dulbecco's phosphate buffered saline and 0.001% poloxamer 188, pH=7.4.

### 4.2 Functional Properties

### 4.2.1 Clearance Time

The disclosure provides a pharmaceutical composition (e.g., diluted formulation or lower ionic strength formulation comprising an AAV comprising an expression cassette encoding a transgene) resulting in a delayed clearance time from the SCS. In some embodiments, a pharmaceutical composition comprising aggregated AAV or comprising more levels of aggregated AAV results in delayed clearance time from the SCS as compared to a pharmaceutical composition comprising lower levels of aggregated AAV, or comprising substantially no aggregated AAV, or comprising no aggregation. In some embodiments, a pharmaceutical composition comprising aggregated AAV or comprising more levels of aggregated AAV results in delayed clearance time from the eye as compared to a pharmaceutical composition comprising lower levels of aggregated AAV, or comprising substantially no aggregated AAV, or comprising no aggregation. In some embodiments, a pharmaceutical composition comprises more aggregated AAV than a reference composition normally used for subretinal injection. In some embodiments, the clearance time of the pharmaceutical composition after the pharmaceutical composition is administered to the SCS is equal to or higher than the clearance time of a reference pharmaceutical composition after the reference pharmaceutical composition is administered subretinally or intravitreously. In some embodiments, the clearance time of the pharmaceutical composition after the pharmaceutical composition is administered to the SCS is equal to or higher than the clearance time of a reference pharmaceutical composition after the reference pharmaceutical composition is administered to the SCS. In some embodiments, the pharmaceutical composition has more aggregated AAV than the level of aggregated AAV in the reference pharmaceutical composition.

In some embodiments, a pharmaceutical composition (e.g., diluted formulation or low ionic strength formulation comprising an AAV comprising an expression cassette encoding a transgene) results in a clearance time from the SCS of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days. In some embodiments, the clearance time from the SCS is of about 3 days to about 365 days, about 3 days to about 300 days, about 3 days to about 200 days, about 3 days to about 150 days, about 3 days to about 125 days, about 7 days to about 365 days, about 7 days to about 300 days, about 7 days to about 200 days, about 7 days to about 150 days, about 7 days to about 125 days. The “clearance time from the SCS” is the time required for substantially all of the pharmaceutical composition, the pharmaceutical agent, or the AAV to escape the SCS. In some embodiments, the “clearance time from the SCS” is the time required for the pharmaceutical composition, the pharmaceutical agent, or the AAV to not be detected in the SCS by any standard method (such as those described in Section 4.6 and Section 5). In some embodiments, the “clearance time from the SCS” is when the pharmaceutical composition, the pharmaceutical agent, or the AAV is present in the SCS in an amount that is at most about 2% or at most about 5% as detected by any standard method (such as those described in Section 4.6 and Section 5).

In some embodiments, the pharmaceutical composition (e.g., diluted formulation or low ionic strength formulation comprising an AAV comprising an expression cassette encoding a transgene) results in a clearance time from the eye of about 30 minutes to about 20 hours, about 2 hours to about 20 hours, about 30 minutes to about 24 hours, about 1 hour to about 2 hours, about 30 minutes to about 90 days, about 30 minutes to about 60 days, about 30 minutes to about 30 days, about 30 minutes to about 21 days, about 30 minutes to about 14 days, about 30 minutes to about 7 days, about 30 minutes to about 3 days, about 30 minutes to about 2 days, about 30 minutes to about 1 day, about 4 hours to about 90 days, about 4 hours to about 60 days, about 4 hours to about 30 days, about 4 hours to about 21 days, about 4 hours to about 14 days, about 4 hours to about 7 days, about 4 hours to about 3 days, about 4 hours to about 2 days, about 4 hours to about 1 day, about 4 hours to about 8 hours, about 4 hours to about 16 hours, about 4 hours to about 20 hours, about 1 day to about 90 days, about 1 day to about 60 days, about 1 day to about 30 days, about 1 day to about 21 days, about 1 day to about 14 days, about 1 day to about 7 days, about 1 day to about 3 days, about 2 days to about 90 days, about 3 days to about 90 days, about 3 days to about 60 days, about 3 days to about 30 days, about 3 days to about 21 days, about 3 days to about 14 days, or about 3 days to about 7 days. In some embodiments, the clearance time from the eye is of about 3 days to about 365 days, about 3 days to about 300 days, about 3 days to about 200 days, about 3 days to about 150 days, about 3 days to about 125 days, about 7 days to about 365 days, about 7 days to about 300 days, about 7 days to about 200 days, about 7 days to about 150 days, about 7 days to about 125 days. The “clearance time from the eye” is the time required for substantially all of the pharmaceutical composition, the pharmaceutical agent, or the AAV to escape the eye. In some embodiments, the “clearance time from the eye” is the time required for the pharmaceutical composition, the pharmaceutical agent, or the AAV to not be detected in the eye by any method (such as those described in Section 4.6 and Section 5). In some embodiments, the “clearance time from the eye” is when the pharmaceutical composition, the pharmaceutical agent, or the AAV is present in the eye in an amount that is at most about 2% or at most about 5% as detected by any standard method (such as those described in Section 4.6 and Section 5).

In some embodiments, the clearance time is not prior to (e.g., the clearance time from the SCS or the eye does not occur before) about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after administration of the pharmaceutical composition (e.g., a liquid formulation). In some embodiments, the clearance time is about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after administration of the pharmaceutical composition (e.g., diluted formulation or low ionic strength formulation).

In some embodiments, a pharmaceutical composition comprising AAV aggregation or a higher level of AAV aggregation (e.g., diluted formulation or low ionic strength formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition (reference pharmaceutical composition) that does not comprise AAV aggregation or comprises a lower level of AAV aggregation is used to administer the AAV comprising the expression cassette encoding the transgene (e.g., via a subretinal administration, intravitreous administration, or to the SCS).

In some embodiments, a suprachoroidal administration of a pharmaceutical composition comprising AAV aggregation or a higher level of AAV aggregation (e.g., diluted formulation or low ionic strength formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition (reference pharmaceutical composition) that does not comprise AAV aggregation or comprises a lower level of AAV aggregation is used, for example, to administer the AAV comprising the expression cassette encoding the transgene by suprachoroidal administration.

In some embodiments, a suprachoroidal administration of a pharmaceutical composition comprising AAV aggregation or a higher level of AAV aggregation (e.g., diluted formulation or low ionic strength formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a pharmaceutical composition (reference pharmaceutical composition) that does not comprise AAV aggregation or comprises a lower level of AAV aggregation is used, for example, to administer the AAV comprising the expression cassette encoding the transgene by subretinal administration or by intravitreous administration.

In some embodiments, a suprachoroidal administration a pharmaceutical composition comprising AAV aggregation or a higher level of AAV aggregation as compared to a reference (e.g., diluted formulation or low ionic strength formulation comprising an AAV comprising an expression cassette encoding a transgene) results in a clearance time that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is used, for example, to administer the AAV comprising the expression cassette encoding the transgene via subretinal administration or via intravitreous administration.

In some embodiments, the clearance time of a pharmaceutical composition comprising aggregated AAV (e.g., a pharmaceutical composition comprising an AAV comprising an expression cassette encoding a transgene) administered by suprachoroidal injection is greater than the clearance time of the same pharmaceutical composition administered via subretinal administration or via intravitreous administration. In some embodiments, the clearance time after a pharmaceutical composition comprising aggregated AAV (e.g., a pharmaceutical composition comprising an AAV comprising an expression cassette encoding a transgene) is administered by suprachoroidal injection is greater than the clearance time after a comparable pharmaceutical composition comprising no AAV aggregation or lower levels of AAV aggregation (e.g., a reference pharmaceutical composition) is administered by suprachoroidal injection. In some embodiments, the clearance time of a pharmaceutical composition comprising aggregated AAV (e.g., a pharmaceutical composition comprising an AAV comprising an expression cassette encoding a transgene) after suprachoroidal injection is greater than the clearance time of a comparable pharmaceutical composition comprising lower levels of AAV aggregation (or no detectable AAV aggregation) after subretinal administration or via intravitreous administration. In some embodiments, the clearance time of a pharmaceutical composition comprising aggregated AAV (e.g., a pharmaceutical composition comprising an AAV comprising an expression cassette encoding a transgene) administered by suprachoroidal injection is greater than a comparable pharmaceutical composition comprising lower levels of AAV aggregation (or no detectable AAV aggregation) administered via subretinal administration or via intravitreous administration.

In some embodiments, the clearance time of a pharmaceutical composition comprising aggregated AAV (e.g., a pharmaceutical composition comprising an AAV comprising an expression cassette encoding a transgene) after suprachoroidal administration is greater than the clearance time of the same pharmaceutical composition after subretinal administration or intravitreous administration by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of a pharmaceutical composition comprising aggregated AAV (e.g., a pharmaceutical composition comprising an AAV comprising an expression cassette encoding a transgene) after suprachoroidal administration is greater than the clearance time after a comparable pharmaceutical composition comprising less aggregated AAV or no detectable aggregated AAV is administered by suprachoroidal injection by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of a pharmaceutical composition comprising aggregated AAV (e.g., a pharmaceutical composition comprising an AAV comprising an expression cassette encoding a transgene) after suprachoroidal administration is greater than the clearance time after a comparable pharmaceutical composition comprising less aggregated AAV or no detectable aggregated AAV is administered by subretinal administration or intravitreous administration by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the clearance time of the pharmaceutical composition administered via intravitreous injection or via subretinal injection is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

In some embodiments, the clearance time of a reference pharmaceutical composition administered by intravitreous injection, subretinal injection, or to the SCS is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

In some embodiments, the clearance time is the clearance time from the eye. In some embodiments, the clearance time is the clearance time from the SCS. In some embodiments, the clearance time is the clearance time from the site of injection.

In some embodiments, the “clearance time from the SCS” is the amount of time required following injection of the pharmaceutical composition, the pharmaceutical agent, or the AAV required for the SCS thickness at or near the site of injection to decrease to about 1 nm or less, about 2 nm or less, about 5 nm or less, about 10 nm or less, about 25 nm or less, about 50 nm or less, about 100 nm or less, about 200 nm or less, or about 500 nm or less, as measured by standard techniques (e.g. in-vivo imaging techniques such as OCT imaging, ultra-high resolution OCT (UHR-OCT), ultrasound and three-dimensional (3D) cryo-reconstruction). In some embodiments, the “clearance time from the SCS” is the amount of time required following injection of the pharmaceutical composition, the pharmaceutical agent, or the AAV required for the SCS thickness at or near the site of injection to decrease to 500 nm or less, about 200 nm or less, about 100 nm or less, about 50 nm or less, about 25 nm or less, about 10 nm or less, or is undetectable, as measured by standard techniques (e.g. in-vivo imaging techniques such as OCT imaging, UHR-OCT, ultrasound and three-dimensional (3D) cryo-reconstruction). In some embodiments, SCS thickness is measured using Heidelberg Optical Coherence Tomography (OCT). In some embodiments, the pharmaceutical composition (e.g., diluted pharmaceutical composition) has an amount of viral vector aggregation sufficient to make the clearance time at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the “clearance time from the SCS” is the amount of time required following injection for the pharmaceutical composition, the pharmaceutical agent, or the AAV required to spread circumferentially from the site of injection to cover about one-sixteenth or more, about one-eighth or more, about one-fourth or more, about one-half or more, about three-fourths or more, or all of the circumference of the choroid, as measured by standard techniques (e.g. in-vivo imaging techniques such as OCT imaging). In some embodiments, the pharmaceutical composition (e.g., diluted pharmaceutical composition) has an amount of viral vector aggregation sufficient to make the clearance time at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

In some embodiments, the pharmaceutical composition (e.g., diluted pharmaceutical composition) is administered to the suprachroidal space of the eye of a pig (e.g., minipig, such as Yucatan minipig). In some embodiments, the pig is a minipig. In some embodiments, the minipig can be Goettingen, Yucatan, Bama Xiang Zhu, Wuzhishan, and/or Xi Shuang Banna. In some embodiments, the minipig is Yucatan. In some embodiments, the pharmaceutical composition (e.g., diluted pharmaceutical composition) has an amount of viral vector aggregation such that, when administered to the SCS of the eye of a pig, the clearance time of the pharmaceutical composition is between about 5 days and about 15 days, about 6 days to about 15 days days, about 7 days to about 15 days, about 8 days to about 15 days, about 9 days to about 15 days, about 10 days to about 15 days, about 11 days to about 15 days, about 12 days to about 15 days, about 13 days to about 15 days, about 14 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, or about 5 days to about 6 days. In some embodiments, the pharmaceutical composition (e.g., diluted pharmaceutical composition) is administered to the suprachroidal space of the eye of a pig. In some embodiments, the pharmaceutical composition (e.g., diluted pharmaceutical composition) has an amount of viral vector aggregation such that, when administered to the SCS of the eye of a pig, the clearance time of the pharmaceutical composition is between about 5 days and about 15 days.

### 4.2.2 Circumferential Spread

In some embodiments, a pharmaceutical composition (e.g., diluted formulation or lower ionic strength formulation) localizes at the site of injection. In some embodiments, a pharmaceutical composition (e.g., diluted formulation or lower ionic strength formulation) localizes at the site of injection for a longer period of time than a reference pharmaceutical composition comprising less AAV aggregation or no detectable AAV aggregation. In some embodiments, a pharmaceutical composition (e.g., diluted formulation or lower ionic strength formulation) localizes at the site of injection for a longer period of time when injected in the SCS as compared to when the pharmaceutical composition is administered by subretinal injection or intravitreous injection. The pharmaceutical composition can have different levels of viral vector aggregation. In some embodiments, a pharmaceutical composition comprising aggregated AAV remains localized in the SCS for a longer period of time as compared to a pharmaceutical composition comprising less AAV aggregation or no detectable AAV aggregation (e.g., a reference pharmaceutical composition).

In some embodiments, localization can be determined by evaluating circumferential spread (e.g., 2D circumferential spread). In some embodiments, circumferential spread is determined by analyzing SCS expansion or opening in the quadrant where the injection was made (in some cases, in 2D this space adjacent to the choroid appears linear). In some embodiments, a pharmaceutical composition comprising aggregated AAV (e.g., diluted formulation or lower ionic strength formulation comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when a reference pharmaceutical composition (e.g., comprising less AAV aggregation or no detectable levels of AAV aggregation) is used to administer the AAV comprising the expression cassette encoding the transgene (e.g., by suprachoroidal injection, by subretinal injection, or by intravitreous injection).

In some embodiments, a suprachoroidal administration of a pharmaceutical composition comprising aggregated AAV (e.g., diluted formulation or lower ionic strength formulation comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when reference pharmaceutical composition (e.g., comprising less AAV aggregation or no detectable levels of AAV aggregation) is used, for example, to administer the AAV comprising the expression cassette encoding the transgene by suprachoroidal administration, by subretinal administration, or by intravitreous administration.

In some embodiments, a suprachoroidal administration of a pharmaceutical composition comprising aggregated AAV (e.g., diluted formulation or lower ionic strength formulation comprising an AAV comprising an expression cassette encoding a transgene) results in a circumferential spread that is at least 2 times less, at least 3 times less, at least 4 times less, at least 5 times less, at least 6 times less, at least 7 times less, at least 8 times less, at least 9 times less, at least 10 times less, at least 15 times less, at least 20 times less, at least 50 times less, at least 100 times less, at least 5% less, at least 10% less, at least 15% less, at least 20% less, at least 25% less, at least 30% less, at least 35% less, at least 40%, at least 45% less, at least 50% less, at least 55% less, at least 60% less, at least 65% less, at least 70% less, at least 75% less, at least 80% less, at least 85% less, at least 90% less, at least 95% less, at least 100% less, at least 150% less, or at least 200% less, at least 250% less, or at least 300%, at least 400% less, or at least 500% less than when the same pharmaceutical composition is used, for example, to administer the AAV comprising the expression cassette encoding the transgene by subretinal administration or by intravitreous administration.

In some embodiments, the circumferential spread can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

In some embodiments, the pharmaceutical composition (e.g., diluted pharmaceutical composition) is administered to the suprachroidal space of the eye of a pig. In some embodiments, the pharmaceutical composition (e.g., diluted pharmaceutical composition) has an amount of viral vector aggregation such that, when administered to the SCS of the eye of a pig, the circumferential spread of the pharmaceutical composition from the site of injection is about one-sixteenth or less, about one-eighth or less, about one-fourth or less, or about one-half or less of a surface of the choroid at a time within about 5 minutes, about 10 minutes, 15 minutes, about 20 minutes, about 30 minutes, about 45 minutes, about one hour, about 2 hours, about 4 hours, about 8 hours, or about 24 hours of administration. In some embodiments, the pharmaceutical composition (e.g., diluted pharmaceutical composition) is administered to the suprachroidal space of the eye of a pig. In some embodiments, the pharmaceutical composition (e.g., diluted pharmaceutical composition) has an amount of viral vector aggregation such that, when administered to the SCS of the eye of a pig, the circumferential spread of the pharmaceutical composition from the site of injection is about about one-eighth or less of a surface of the choroid at a time within about 5 minutes, about 10 minutes, 15 minutes, about 20 minutes, about 30 minutes, about 45 minutes, about one hour, about 2 hours, about 4 hours, about 8 hours, or about 24 hours of administration. In some embodiments, the pharmaceutical composition (e.g., diluted pharmaceutical composition) has an amount of viral vector aggregation such that, when administered to the SCS of the eye of a pig, the circumferential spread of the pharmaceutical composition from the site of injection is about one-eighth or less of a surface of the choroid at a time within about one hour of administration.

### 4.2.3 SCS Thickness

In some embodiments, localization can be determined by evaluating SCS thickness after a pharmaceutical composition (e.g., diluted formulation or lower ionic strength formulation) is administered to a subject. In some embodiments, a pharmaceutical composition (e.g., diluted formulation or lower ionic strength formulation) increases the thickness of the SCS after the pharmaceutical composition (e.g., diluted formulation or lower ionic strength formulation) is injected in the SCS. In some embodiments, the infusion into the SCS of a pharmaceutical composition comprising aggregated AAV (e.g., diluted formulation or lower ionic strength formulation) can expand SCS thickness beyond the SCS thickness achieved when a reference pharmaceutical composition (e.g., comprising lower levels of AAV aggregation or comprising no detectable AAV aggregation) is infused into the SCS. In some embodiments, increasing the SCS thickness with a pharmaceutical composition comprising aggregated AAV (e.g., diluted formulation or lower ionic strength formulation) may ease access to the SCS, thereby easing or permitting the disposal of a device in the SCS. In some embodiments, expanding the SCS thickness allows for the pharmaceutical composition (e.g., diluted formulation or lower ionic strength formulation) and/or the AAV encoded transgene to remain at the site of injection (localized) for a longer period of time. In some embodiments, a pharmaceutical composition comprising aggregated AAV increases the thickness at or near the site of injection for a longer period of time as compared to a reference pharmaceutical composition. In some embodiments, a pharmaceutical composition comprising aggregated AAV increases the thickness at or near the site of injection for a longer period of time as compared to a pharmaceutical composition comprising less AAV aggregation or no detectable level of AAV aggregation. In some embodiments, the thickness at the site of injection after the pharmaceutical composition is administered to the SCS is equal to or higher than the thickness at the site of injection of a reference pharmaceutical composition after the reference pharmaceutical composition is administered subretinally or intravitreously. In some embodiments, the thickness at the site of injection of the pharmaceutical composition after the pharmaceutical composition is administered to the SCS is equal to or higher than the thickness at the site of injection of a reference pharmaceutical composition after the reference pharmaceutical composition is administered to the SCS.

In some embodiments, a suprachoroidal administration of a pharmaceutical composition comprising aggregated AAV (e.g., diluted formulation or lower ionic strength formulation comprising an AAV comprising an expression cassette encoding a transgene) results in an increase in the SCS thickness that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a reference pharmaceutical composition (comprising lower levels of AAV aggregation or no detectable AAV aggregation) is used, for example, to administer the AAV comprising the expression cassette encoding the transgene by suprachoroidal administration.

In some embodiments, a suprachoroidal administration of a pharmaceutical composition comprising AAV aggregation (e.g., diluted formulation or lower ionic strength formulation comprising an AAV comprising an expression cassette encoding a transgene) results in an increase in thickness at or near the site of injection that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a reference pharmaceutical composition (comprising lower levels of AAV aggregation or no detectable AAV aggregation) is used, for example, to administer the AAV comprising the expression cassette encoding the transgene by subretinal administration or by intravitreous administration.

In some embodiments, a suprachoroidal administration of a pharmaceutical composition comprising AAV aggregation (e.g., diluted formulation or lower ionic strength formulation comprising an AAV comprising an expression cassette encoding a transgene) results in an increase in thickness at or near the site of injection that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is used, for example, to administer the AAV comprising the expression cassette encoding the transgene by subretinal administration or by intravitreous administration.

In some embodiments, the thickness obtained at the site of injection after a pharmaceutical composition comprising aggregated AAV (e.g., a pharmaceutical composition comprising an AAV comprising an expression cassette encoding a transgene) is administered by suprachoroidal injection is greater than after a reference pharmaceutical composition is administered by suprachoroidal injection. In some embodiments, the thickness obtained at the site of injection after a pharmaceutical composition comprising aggregated AAV (e.g., a pharmaceutical composition comprising an AAV comprising an expression cassette encoding a transgene) is administered by suprachoroidal injection is greater than after a reference pharmaceutical composition is administered by subretinal injection or by intravitreous injection. In some embodiments, the thickness obtained at the site of injection after a pharmaceutical composition comprising aggregated AAV (e.g., a pharmaceutical composition comprising an AAV comprising an expression cassette encoding a transgene) is administered by suprachoroidal injection is greater than after the same pharmaceutical composition is administered by subretinal administration or by intravitreous administration.

In some embodiments, the thickness at or near the site of injection (e.g., thickness at or near the SCS) can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

In some embodiments, the pharmaceutical composition (e.g., diluted pharmaceutical composition) is administered to the suprachroidal space of the eye of a pig. In some embodiments, the pharmaceutical composition (e.g., diluted pharmaceutical composition) has an amount of viral vector aggregation such that, when administered to the SCS of the eye of a pig, the thickness of the SCS at the site of injection is between about 400 and about 800 μm, about 400 μm to about 700 μm, about 400 μm to about 600 μm, about 400 μm to about 500 μm, about 500 μm to about 800 μm, about 600 μm to about 800 μm, 700 μm to about 800 μm at a time at a time within about 5 minutes, about 10 minutes, 15 minutes, about 20 minutes, about 30 minutes, about 45 minutes, about one hour, about 2 hours, about 4 hours, about 8 hours, or about 24 hours of administration. In some embodiments, the pharmaceutical composition (e.g., diluted pharmaceutical composition) is administered to the suprachroidal space of the eye of a pig. In some embodiments, the pharmaceutical composition (e.g., diluted pharmaceutical composition) has an amount of viral vector aggregation such that, when administered to the SCS of the eye of a pig, the thickness of the SCS at the site of injection is between about 400 and about 800 μm at a time within about 5 minutes, about 10 minutes, 15 minutes, about 20 minutes, about 30 minutes, about 45 minutes, about one hour, about 2 hours, about 4 hours, about 8 hours, or about 24 hours of administration. In some embodiments, the pharmaceutical composition (e.g., diluted pharmaceutical composition) has an amount of viral vector aggregation such that, when administered to the SCS of the eye of a pig, the thickness of the SCS at the site of injection is between about 400 and about 800 μm at a time within about one hour of administration.

### 4.2.4 Rate of Transduction (Rate of Infection) at Site of Injection

In some embodiments, the rate of transduction (rate of infection) at the site of injection after a pharmaceutical composition is administered in the SCS is equal to or higher as compared to the rate of transduction (rate of infection) at a site of injection after the same pharmaceutical composition is administered via a subretinal administration or via an intravenous administration. In some embodiments, the rate of transduction (rate of infection) at the site of injection after a pharmaceutical composition is administered in the SCS is equal to or higher as compared to the rate of transduction (rate of infection) at the site of injection after a reference pharmaceutical composition is administered via a subretinal, or intravenous administration, or to the SCS. In some embodiments, the pharmaceutical composition has higher levels of AAV aggregation than the reference pharmaceutical composition. In some embodiments, the pharmaceutical composition and the reference pharmaceutical composition have the same vector genome concentration. In some embodiments, the pharmaceutical composition and the reference pharmaceutical composition have the same amount of genome copies. In some embodiments, the rate of transduction (rate of infection) at the site of injection after the pharmaceutical composition is administered to the SCS is increased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500% as compared to the rate of transduction (rate of infection) at the site of injection after the same pharmaceutical composition is administered via subretinal or via intravitreous administrations. In some embodiments, the rate of transduction (rate of infection) at the site of injection after the pharmaceutical composition is administered to the SCS is increased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500% as compared to the rate of transduction (rate of infection) at the site of injection after a reference pharmaceutical composition is administered to the SCS, or via subretinal, or via intravitreous administrations. In some embodiments, the pharmaceutical composition has a higher level of AAV aggregation as compared to the reference pharmaceutical composition.

In some embodiments, a level of AAV at the site of injection is equal to or higher after the pharmaceutical composition is administered suprachoroidally as compared to a level of AAV at the site of injection after the same pharmaceutical composition is administered via a subretinal administration or via an intravenous administration. In some embodiments, a level of AAV at the site of injection after the pharmaceutical composition is administered suprachoroidally is equal to or higher as compared to a level of AAV at the site of injection after a reference pharmaceutical composition is administered via a subretinal, or intravenous administration, or to the SCS. In some embodiments, the pharmaceutical composition has a higher level of AAV aggregation than the reference pharmaceutical composition. In some embodiments, the pharmaceutical composition and the reference pharmaceutical composition have the same vector genome concentration. In some embodiments, the pharmaceutical composition and a reference pharmaceutical composition have the same amount of genome copies. In some embodiments, the increase in the level of AAV at the site of injection is an increase of at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

In some embodiments, the level of AAV at the site of injection after the pharmaceutical composition is administered to the SCS is increased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500% as compared to the level of AAV at the site of injection after the same pharmaceutical composition is administered via subretinal or via intravitreous administrations. In some embodiments, the AAV level at the site of injection after the pharmaceutical composition is administered to the SCS is increased by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500% as compared to the AAV level at the site of injection after a reference pharmaceutical composition is administered to the SCS, or via subretinal, or via intravitreous administrations. In some embodiments, the pharmaceutical composition has a higher level of AAV aggregation as compared to the reference pharmaceutical composition.

In some embodiments, the AAV level or the rate of transduction (rate of infection) is determined about 30 minutes, 1 hour, 2 hours, 4 hours, 6 hours, 8 hours, 10 hours, 12 hours, 14 hours, 15 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or at most about 400 days after administration.

### 4.2.5 Transgene Expression

In some embodiments, the concentration of a transgene product is at least equal to or higher after a pharmaceutical composition is injected in the SCS as compared to after a reference pharmaceutical composition is injected in the SCS. In some embodiments, the concentration of a transgene product is at least equal to or higher after a pharmaceutical composition is injected in the SCS as compared to after a reference pharmaceutical composition is injected by subretinal injection or by intravitreous injection. In some embodiments, the concentration of a transgene product is at least equal to or higher after a pharmaceutical composition is injected in the SCS as compared to after the same pharmaceutical composition is injected by subretinal injection or by intravitreous injection.

In some embodiments, a transgene product (e.g., concentration of the transgene product) is detected in an eye (e.g., vitreous humor) for a longer period of time after a pharmaceutical composition is injected in the SCS as compared to after a reference pharmaceutical composition is injected in the SCS. In some embodiments, a transgene product (e.g., concentration of the transgene product) is detected in an eye (e.g., vitreous humor) for a longer period of time after a pharmaceutical composition is injected in the SCS as compared to after a reference pharmaceutical composition is injected by subretinal injection or by intravitreous administration. In some embodiments, a transgene product (e.g., concentration of the transgene product) is detected in an eye (e.g., vitreous humor) for a longer period of time after a pharmaceutical composition is injected in the SCS as compared to after the same pharmaceutical composition is injected by subretinal injection or by intravitreous injection.

In some embodiments, the longer period of time is at least about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days longer. In some embodiments, the longer period of time is about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days longer.

In some embodiments, the transgene is detected in an eye (e.g., vitreous humor) for period of time, after the pharmaceutical composition is administered in the SCS, that is at least about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the administration.

In some embodiments, the transgene is detected in an eye (e.g., vitreous humor) for a period of time (e.g., after the reference pharmaceutical composition is administered via subretinal administration or via intravitreous administration or to the SCS; or after the pharmaceutical composition is administered via subretinal or via intravitreous administration) that is at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days after administration.

In some embodiments, the concentration of a transgene product in an eye (e.g., vitreous humor) can be determined 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days after the pharmaceutical composition or the reference pharmaceutical composition is administered.

In some embodiments, a suprachoroidal administration of a pharmaceutical composition (e.g., diluted formulation or lower ionic strength formulation comprising an AAV comprising an expression cassette encoding a transgene) results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than after a reference pharmaceutical composition is used, for example, to administer the AAV comprising the expression cassette encoding the transgene by suprachoroidal administration.

In some embodiments, a suprachoroidal administration of a pharmaceutical composition (e.g., diluted formulation or lower ionic strength formulation comprising an AAV comprising an expression cassette encoding a transgene) results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when a reference pharmaceutical composition (comprising lower levels of AAV aggregation or no detectable AAV aggregation) is used, for example, to administer the AAV comprising the expression cassette encoding the transgene by subretinal administration or by intravitreous administration.

In some embodiments, a suprachoroidal administration of a pharmaceutical composition (e.g., diluted formulation or lower ionic strength formulation comprising an AAV comprising an expression cassette encoding a transgene) results in a higher concentration of the transgene that is at least 2 times greater, at least 3 times greater, at least 4 times greater, at least 5 times greater, at least 6 times greater, at least 7 times greater, at least 8 times greater, at least 9 times greater, at least 10 times greater, at least 15 times greater, at least 20 times greater, at least 50 times greater, at least 100 times greater, at least 5% greater, at least 10% greater, at least 15% greater, at least 20% greater, at least 25% greater, at least 30% greater, at least 35% greater, at least 40%, at least 45% greater, at least 50% greater, at least 55% greater, at least 60% greater, at least 65% greater, at least 70% greater, at least 75% greater, at least 80% greater, at least 85% greater, at least 90% greater, at least 95% greater, at least 100% greater, at least 150% greater, or at least 200% greater, at least 250% greater, or at least 300%, at least 400% greater, or at least 500% greater than when the same pharmaceutical composition is administered via subretinal administration or via intravitreous administration.

In some embodiments, the concentration of the transgene product after a pharmaceutical composition (e.g., a pharmaceutical composition comprising an AAV comprising an expression cassette encoding a transgene) is administered by suprachoroidal injection is greater than after a reference pharmaceutical composition is administered by suprachoroidal injection. In some embodiments, the concentration of the transgene product after a pharmaceutical composition (e.g., a pharmaceutical composition comprising an AAV comprising an expression cassette encoding a transgene) is administered by suprachoroidal injection is greater than after a reference pharmaceutical composition is administered by subretinal administration or via intravitreous administration,

In some embodiments, the pharmaceutical compositions disclosed herein (e.g., a pharmaceutical composition comprising an AAV comprising an expression cassette encoding a transgene) provide greater transgene expression and/or tissue/cell transduction at the back of the eye (e.g., retina) than in the outer layer of the eye (e.g., sclera) through SCS delivery. Such features of the presently disclosed pharmaceutical compositions are advantageous because subretinal delivery is not required to achieve higher expression of the ocular transgenes at the back of the eye than the outer layer of the eye by using the presently disclosed pharmaceutical compositions subretinally.

In some embodiments, the concentration of a transgene product (TP) is equal to or higher in the retina after a presently disclosed pharmaceutical composition comprising AAV encoding the TP is injected in the SCS than a reference pharmaceutical composition comprising the same AAV is injected in the SCS. In other embodiments, the concentration of a TP is equal to or higher in the retina and the concentration of the TP is lower in the sclera after a pharmaceutical composition comprising AAV encoding the TP is injected in the SCS as compared to a reference pharmaceutical composition comprising the same AAV is injected in the SCS.

### 4.2.6 Other Functional Properties

In some embodiments, the pharmaceutical composition described herein has a desired level of AAV aggregation that is suitable for suprachoroidal injection. In some embodiments, the recombinant AAV in the pharmaceutical composition is at least as stable as the recombinant AAV in a reference pharmaceutical composition (or a comparable pharmaceutical composition). In some embodiments, the recombinant AAV in the pharmaceutical composition is at least 50% as stable as the recombinant AAV in a reference pharmaceutical composition (or a comparable pharmaceutical composition). In some embodiments, the recombinant AAV in the pharmaceutical composition has at least the same or a comparable infectivity level as the recombinant AAV in a reference pharmaceutical composition. In some embodiments, the recombinant AAV in the pharmaceutical composition has at least the same or a comparable free DNA level as the recombinant AAV in a reference pharmaceutical composition. In some embodiments, the recombinant AAV in the pharmaceutical composition has at least the same or a comparable in vitro relative potency (IVRP) as the recombinant AAV in a reference pharmaceutical composition. In some embodiments, the recombinant AAV in the pharmaceutical composition has at least the same or a comparable change in size level as the recombinant AAV in a reference pharmaceutical composition.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable to freeze/thaw cycles than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable to freeze/thaw cycles as the same recombinant AAV in a reference pharmaceutical composition. In certain embodiments, the stability of the recombinant AAV is determined by an assay or assays disclosed in Section 4.6 and Section 5.

In certain embodiments, the recombinant AAV in the pharmaceutical composition exhibits at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more infectivity than the same recombinant AAV in a reference pharmaceutical composition. In some embodiments, the recombinant AAV in the pharmaceutical composition has at least about 50%, 55%, 60%, 65%, 70%, 75%, 80%, 85%, 90%, 95%, or 100% the infectivity of the same recombinant AAV in a reference pharmaceutical composition. In certain embodiments, the virus infectivity of the recombinant AAV is determined by an assay or assays disclosed in the present disclosure. In certain embodiments, the size of the recombinant AAV is determined by an assay or assays disclosed in Section 4.6 and Section 5. In certain embodiments, the size is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable over a period of time (e.g., when stored at −20° C. or at 37° C.), for example, at least about or about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, 12 months, about 15 months, about 18 months, about 24 months, about 2 years, about 3 years, about 4 years than the same recombinant AAV in a reference pharmaceutical composition. In some embodiments, the recombinant AAV in the pharmaceutical composition is at least about 50%, 55%, 60%, 65%, 70%, 75%, 80%, 85%, 90%, 95%, or 100% as stable over a period of time as the same recombinant AAV in a reference pharmaceutical composition. In certain embodiments, the stability over a period of time of the recombinant AAV is determined by an assay or assays disclosed in the present disclosure. In certain embodiments, the stability over a period of time of the recombinant AAV is determined by an assay or assays disclosed in Section 4.6 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition (e.g., when stored at −20° C. or at 37° C.). In some embodiments, the recombinant AAV in the pharmaceutical composition has about the same in vitro relative potency (IVRP) as the same recombinant AAV in a reference pharmaceutical composition. In some embodiments, the recombinant AAV in the pharmaceutical composition has about 50%, 55%, 60%, 65%, 70%, 75%, 80%, 85%, 90%, 95%, or 100% in vitro relative potency (IVRP) as the same recombinant AAV in a reference pharmaceutical composition. In certain embodiments, the in vitro relative potency (IVRP) of the recombinant AAV is determined by an assay or assays disclosed in the present disclosure. In certain embodiments, the in vitro relative potency (IVRP) is measured prior to or after freeze/thaw cycles. In certain embodiments, the in vitro relative potency (IVRP) of the recombinant AAV is determined by an assay or assays disclosed in Section 4.6.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times less free DNA than the same recombinant AAV in a reference pharmaceutical composition. In some embodiments, the recombinant AAV in the pharmaceutical composition has about the same amount of free DNA as the same recombinant AAV in a reference pharmaceutical composition. In some embodiments, the recombinant AAV in the pharmaceutical composition has about not more than two times the amount of free DNA as the same recombinant AAV in a reference pharmaceutical composition. In some embodiments, the recombinant AAV in the pharmaceutical composition has about 50%, 55%, 60%, 65%, 70%, 75%, 80%, 85%, 90%, 95%, or 100% the amount of free DNA as the same recombinant AAV in a reference pharmaceutical composition. In some embodiments, the recombinant AAV in the pharmaceutical composition has at least about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, or about 3 times less free DNA than the same recombinant AAV in a reference pharmaceutical composition. In certain embodiments, the free DNA of the recombinant AAV is determined by an assay or assays disclosed in Section 4.6 and Section 5.

In certain embodiments, the recombinant AAV in the pharmaceutical composition has at most 20%, 15%, 10%, 8%, 5%, 4%, 10%, 2%, or 1% change in size over a period of time (e.g., when stored at −20° C. or at 37° C.), for example, at least about or about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in the present disclosure. In certain embodiments, the size is measured prior to or after freeze/thaw cycles. In certain embodiments, the size of the recombinant AAV is determined by an assay or assays disclosed in Section 4.6.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 times more stable than the same recombinant AAV in a reference pharmaceutical composition (e.g., when stored at −20° C. or at 37° C.). In some embodiments, the recombinant AAV in the pharmaceutical composition is about as stable as the same recombinant AAV in a reference pharmaceutical composition (e.g., when stored at −20° C. or at 37° C.). In some embodiments, the recombinant AAV in the pharmaceutical composition is at least about 50%, 55%, 60%, 65%, 70%, 75%, 80%, 85%, 90%, 95%, or 99% as stable as the same recombinant AAV in a reference pharmaceutical composition (e.g., when stored at −20° C. or at 37° C.). In certain embodiments, the stability of the recombinant AAV is determined by an assay or assays disclosed in Section 4.6.

In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months without loss of stability as determined, e.g. by an assay or assays disclosed in Section 4.6 or. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at ≤60° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at −80° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. after having been stored at −20° C. for 1, 2, 3, 4, 5, 6, 7, 8, 9, 10, or 12 months without loss of stability.

In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at −80° C., then being thawed and, after thawing, being stored at 2-10° C., 4-8° C., 2° C., 3° C., 4° C., 5° C., 6° C., 7° C., 8° C. or 9° C. for 1, 2, 3, 4, 5, 6, 7, 8, 9, 10, or 12 additional months without loss of stability as determined, e.g., by an assay or assays disclosed in Section 4.6 or. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at −80° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.6 or 5. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, or 24 months at ≤60° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.6 or 5.

Effects of the methods or pharmaceutical compositions provided herein may be monitored by measuring signs of vision loss, infection, inflammation and other safety events, including retinal detachment. In some embodiments, different pharmaceutical compositions (e.g., diluted formulation or lower ionic strength formulation) having different AAV aggregation levels can be used to deliver the vector in the SCS. In some embodiments, vectors delivered using a pharmaceutical composition comprising aggregated AAV (e.g., diluted formulation or lower ionic strength formulation) are more effective than vectors delivered using a reference pharmaceutical composition (e.g., when administered in the SCS). In some embodiments, vectors delivered using a formulation comprising aggregated AAV results in improved vision as compared to vectors delivered using a formulation comprising lower levels of aggregated AAV or no detectable level of aggregated AAV.

Effects of the methods or pharmaceutical compositions provided herein may also be measured by a change from baseline in National Eye Institute Visual Functioning Questionnaire, the Rasch-scored version (NEI-VFQ-28-R) (composite score; activity limitation domain score; and socio-emotional functioning domain score). In some embodiments, effects of the methods provided herein may also be measured by a change from baseline in National Eye Institute Visual Functioning Questionnaire 25-item version (NEI-VFQ-25) (composite score and mental health subscale score). In some embodiments, effects of the methods provided herein may also be measured by a change from baseline in Macular Disease Treatment Satisfaction Questionnaire (MacTSQ) (composite score; safety, efficacy, and discomfort domain score; and information provision and convenience domain score).

In specific embodiments, the efficacy of a method or vector (vector formulation) described herein is reflected by an improvement in vision at about 4 weeks, 12 weeks, 6 months, 12 months, 24 months, 36 months, or at other desired timepoints. In a specific embodiment, the improvement in vision is characterized by an increase in BCVA, for example, an increase by 1 letter, 2 letters, 3 letters, 4 letters, 5 letters, 6 letters, 7 letters, 8 letters, 9 letters, 10 letters, 11 letters, or 12 letters, or more. In a specific embodiment, the improvement in vision is characterized by a 5%, 10%, 15%, 20%, 30%, 40%, 50% or more increase in visual acuity from baseline.

In specific embodiments, there is no inflammation in the eye after treatment or little inflammation in the eye after treatment (for example, an increase in the level of inflammation by 10%, 5%, 2%, 1% or less from baseline).

### 4.2.7 Reduction of AAV Empty Particles and Capsids

In some embodiments, provided herein is a pharmaceutical composition of the disclosure comprising a recombinant AAV vector comprising an expression cassette encoding a transgene, and comprising low or undetectable levels of AAV empty capsids or AAV empty particles. In some embodiments, a pharmaceutical composition of the disclosure comprises lower amounts of AAV empty capsids or AAV empty particles as compared to a reference pharmaceutical composition. In some embodiments, the amount of the AAV empty capsids or AAV empty particles in the pharmaceutical composition is lower by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% as compared to the amount of the AAV empty capsids or AAV empty particles in a reference pharmaceutical composition. In some embodiments, the amount of the AAV empty capsids or AAV empty particles in the pharmaceutical composition is lower by about or at least about 1 fold, 2 folds, 3 folds, 4 folds, 5 folds, 6 folds, 7 folds, 8 folds, 9 folds, 10 folds, 15 folds, 20 folds, 25 folds, 30 folds, 40 folds, 45 folds, 50 folds, 55 folds, 60 folds, 65 folds, 70 folds, 75 folds, 80 folds, 85 folds, 90 folds, 95 folds, 100 folds, or more than 100 folds as compared to the amount of the AAV empty capsids or AAV empty particles in a reference pharmaceutical composition. In some embodiments, the pharmaceutical composition of the disclosure comprising reduced or undetectable AAV empty capsids or AAV empty particles comprises an ionic strength of about or at most about 5 mM, 10 mM, 15 mM, 20 mM, 25 mM, 30 mM, 35 mM, 40 mM, 45 mM, 50 mM, 55 mM, or 60 mM. In some embodiments, the reference pharmaceutical composition comprises an ionic strength of more than about 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, or more than about 200 mM.

In some embodiments, disclosed herein is a method of reducing or eliminating AAV empty capsids or AAV empty particles. In some embodiments, the method comprises introducing a solution comprising an ionic strength of at most about 60 mM to a formulation comprising a mixture of a recombinant adeno-associated virus (AAV) vector of the disclosure and AAV empty capsids or AAV empty particles. In some embodiments, introducing a solution comprising an ionic strength of at most about 60 mM causes the rAAV of the disclosure to aggregate. In some embodiments, introducing a solution comprising an ionic strength of at most about 60 mM does not result in aggregation of the AAV empty capsids or AAV empty particles. In some embodiments, the method comprises removing the AAV empty capsids or AAV empty particles from the formulation after the solution comprising an ionic strength of at most about 60 mM is added to the formulation. In some embodiments, the formulation is then prepared into a pharmaceutical composition. In some embodiments, the pharmaceutical composition comprises the recombinant adeno-associated virus (AAV) vector, wherein the recombinant AAV vector comprises an expression cassette encoding a transgene, and wherein the pharmaceutical composition is suitable for administration to the suprachoroidal space (SCS) of an eye of a human subject. In some embodiments, the method further comprises introducing another solution comprising an ionic strength of at least about 80 mM to the formulation after the empty AAV capsids or empty AAV particles are removed from the formulation. In some embodiments, the another solution comprises an ionic strength of about or at least about 150 mM.

In some embodiments, disclosed herein is a method of reducing or eliminating AAV empty capsids or AAV empty particles in a population of AAV particles. In some embodiments, the population of AAV particles comprises empty AAV particles and AAV particles comprising an expression cassette encoding a transgene. In some embodiments, the method comprises incubating the population of AAV particles in a solution at an ionic strength of at most about 60 mM. In some embodiments, incubating the population of AAV particles in a low ionic strength solution (e.g., about or less than about 60 mM) causes the AAV particles comprising the expression cassette encoding the transgene to aggregate while the empty AAV particles or empty AAV capsids remain unaggregated. In some embodiments, the method comprises removing at least a portion of the empty AAV particles from the population of AAV particles. In some embodiments, the method further comprises introducing another solution comprising an ionic strength of at least about 80 mM to the formulation after the empty AAV capsids or empty AAV particles are removed from the formulation. In some embodiments, the another solution comprises an ionic strength of about or at least about 150 mM.

In some embodiments, the amount of the AAV empty capsids or AAV empty particles in the formulation is reduced by about or at least about 5%, 10%, 15%, 20%, 25%, 30%, 35%, 40%, 45%, 50%, 55%, 60%, 65%, 70%, 75% 80%, 95%, 96%, 97%, 98%, 99%, or 100% after the AAV empty capsids or AAV empty particles are removed from the formulation or solution as compared to the amount of the AAV empty capsids or AAV empty particles in the formulation or solution prior to adding a solution comprising low ionic strength (e.g., a solution comprising at most about 60 mM). In some embodiments, the solution or solution comprising low ionic strength is a solution comprising an ionic strength of about 30 to about 50 mM. In some embodiments, the solution or solution comprising low ionic strength is a solution comprising an ionic strength of about 15 to about 50 mM. In some embodiments, the solution or solution comprising low ionic strength is a solution comprising an ionic strength of about or at most about 50 mM.

In some embodiments, the pharmaceutical composition is free or substantially free of AAV empty capsids or AAV empty particles. The term “free of AAV empty capsids or AAV empty particles” refers to a pharmaceutical composition or formulation in which the level of AAV empty capsids or AAV empty particles is undetectable by a conventional method or an available method. The term “substantially free of AAV empty capsids or AAV empty particles” refers to a pharmaceutical composition or formulation in which the level of AAV empty capsids or AAV empty particles is at most about 5% of the AAV particles in a composition or solution.

### 4.3 Dosage and Mode of Administration

In one aspect, provided herein is a method of suprachoroidal administration for treating a pathology of the eye, comprising administering to the suprachoroidal space in the eye of a human subject in need of treatment a recombinant viral vector comprising a nucleotide sequence encoding a therapeutic product such that the therapeutic product is expressed and results in treatment of the pathology of the eye. In certain embodiments, the administering step is by injecting the recombinant viral vector into the suprachoroidal space using a suprachoroidal drug delivery device. In certain embodiments, the suprachoroidal drug delivery device is a microinjector. In some embodiments, a pharmaceutical composition provided herein is suitable for administration by one, two or more routes of administration (e.g., suitable for suprachoroidal and subretinal administration).

In certain embodiments, the vector genome concentration (VGC) of the pharmaceutical composition (or the reference pharmaceutical composition) is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 2.5×10¹² GC/mL, about 3×10¹² GC/mL, about 5×10¹² GC/mL, about 1.5×10¹³ GC/mL, about 2×10¹³ GC/mL or about 3×10¹³ GC/mL.

In certain embodiments, the vector genome concentration (VGC) of the pharmaceutical composition (or the reference pharmaceutical composition) is about 3×10⁹ GC/mL, 4×10⁹ GC/mL, 5×10⁹ GC/mL, 6×10⁹ GC/mL, 7×10⁹ GC/mL, 8×10⁹ GC/mL, 9×10⁹ GC/mL, about 1×10¹⁰ GC/mL, about 2×10¹⁰ GC/mL, about 3×10¹⁰ GC/mL, about 4×10¹⁰ GC/mL, about 5×10¹⁰ GC/mL, about 6×10¹⁰ GC/mL, about 7×10¹⁰ GC/mL, about 8×10¹⁰ GC/mL, about 9×10¹⁰ GC/mL, about 1×10¹¹ GC/mL, about 2×10¹¹ GC/mL, about 3×10¹¹ GC/mL, about 4×10¹¹ GC/mL, about 5×10¹¹ GC/mL, about 6×10¹¹ GC/mL, about 7×10¹¹ GC/mL, about 8×10¹¹ GC/mL, about 9×10¹¹ GC/mL, about 1×10¹² GC/mL, about 2×10¹² GC/mL, about 3×10² GC/mL, about 4×10¹² GC/mL, about 5×10¹² GC/mL, about 6×10¹² GC/mL, about 7×10¹² GC/mL, about 8×10¹² GC/mL, about 9×10¹² GC/mL, about 1×10¹³ GC/mL, about 1.5×10¹³ GC/mL, about 2×10¹³ GC/mL, about 3×10¹³ GC/mL.

In some embodiments, the volume of the pharmaceutical composition (e.g., diluted formulation or lower ionic strength formulation) is any volume capable of reducing the minimum force to separate the sclera and choroid. In some embodiments, the volume of the pharmaceutical composition (e.g., diluted formulation or lower ionic strength formulation) is about 50 μL to about 1000 μL, 50 μL to about 500 μL, 50 μL to about 400 μL, 50 μL to about 350 μL, 50 μL to about 300 μL, about 50 μL to about 275 μL, about 50 μL to about 250 μL, about 50 μL to about 225 μL, about 50 μL to about 200 μL, about 50 μL to about 175 μL, about 50 μL to about 150 μL, about 60 μL to about 140 μL, about 70 μL to about 130 μL, about 80 μL to about 120 μL, about 90 μL to about 110 μL, or about 100 μL.

Currently available technologies for suprachoroidal space (SCS) delivery exist. Preclinically, SC injections have been achieved with scleral flap technique, catheters and standard hypodermic needles, as well as with microneedles. A hollow-bore 750 μm-long microneedle (Clearside Biomedical, Inc.) can be inserted at the pars, and has shown promise in clinical trials. A microneedle designed with force-sensing technology can be utilized for SC injections, as described by Chitnis, et al. (Chitnis, G. D., et al. A resistance-sensing mechanical injector for the precise delivery of liquids to target tissue. Nat Biomed Eng 3, 621-631 (2019). https://doi.org/10.1038/s41551-019-0350-2). Oxular Limited is developing a delivery system (Oxulumis) that advances an illuminated cannula in the suprachoroidal space. The Orbit device (Gyroscope) is a specially-designed system enabling cannulation of the suprachoroidal space with a flexible cannula. A microneedle inside the cannula is advanced into the subretinal space to enable targeted dose delivery. Ab interno access to the SCS can also be achieved using micro-stents, which serve as minimally-invasive glaucoma surgery (MIGS) devices. Examples include the CyPass® Micro-Stent (Alcon, Fort Worth, Texas, US) and iStent® (Glaukos), which are surgically implanted to provide a conduit from the anterior chamber to the SCS to drain the aqueous humor without forming a filtering bleb. Other devices contemplated for suprachoroidal delivery include those described in UK Patent Publication No. GB 2531910A and U.S. Pat. No. 10,912,883 B2.

In some embodiments, the suprachoroidal drug delivery device is a syringe with a 1 millimeter 30 gauge needle. In some embodiments, the syringe has a larger circumference (e.g., 29 gauge needle). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications.

In some embodiments, a microneedle or syringe is selected based on the level of AAV aggregation of a pharmaceutical composition (e.g., diluted formulation or lower ionic strength formulation). In some embodiments, a microneedle is selected based on the pressure resulted in the eye (e.g., in the SCS) when a pharmaceutical composition (e.g., diluted formulation or lower ionic strength formulation) is administered. For example, a pharmaceutical composition (e.g., diluted formulation or lower ionic strength formulation) having higher levels of AAV aggregation may benefit from the use of a wider microneedle for injection. In some embodiments, the pressure in the SCS is lower when a wider microneedle is used as compared to the pressure obtained when a narrower microneedle is used. In some embodiments, 10 gauge needle, 11 gauge needle, 12 gauge needle, 13 gauge needle, 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, 27 gauge needle, 28 gauge needle, 29 gauge needle, 30 gauge needle, 31 gauge needle, 32 gauge needle, 33 gauge needle, or 34 gauge needle is used. In some embodiments, a 27 gauge needle is used. In some embodiments, a 28 gauge needle is used. In some embodiments, a 29 gauge needle is used. In some embodiments, a 30 gauge needle is used. In some embodiments, a 31 gauge needle is used. In some embodiments, a gauge that is smaller than a 27 gauge needle is used. In some embodiments, a gauge that is larger than a 27 gauge needle is used. In some embodiments, a gauge that is smaller than a 30 gauge needle is used. In some embodiments, a gauge that is higher than a 30 gauge needle is used.

In some embodiments, the pressure during administration of a pharmaceutical composition is about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure during administration of a pharmaceutical composition is not greater than about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure to open the SCS during administration of a pharmaceutical composition is not greater than about 10 PSI, 15 PSI, 20 PSI, 25 PSI, 30 PSI, 35 PSI, 40 PSI, 45 PSI, 50 PSI, 55 PSI, 60 PSI, 65 PSI, 70 PSI, 75 PSI, 80 PSI, 85 PSI, 90 PSI, 95 PSI, 100 PSI, 150 PSI, or 200 PSI. In some embodiments, the pressure during administration of a pharmaceutical composition (or the pressure required to open the SCS) is between 20 PSI and 50 PSI, 20 PSI and 75 PSI, 20 PSI and 40 PSI, 10 PSI and 40 PSI, 10 PSI and 100 PSI, or 10 PSI and 80 PSI. In some embodiments, the pressure decreases as the rate of injection decreases (e.g., pressure decreases from a 4 seconds rate of injection to a 10 seconds rate of injection). In some embodiments, the pressure decreases as the size of the needle increases. In some embodiments, the pressure increases as the level of AAV aggregation increases.

Doses that maintain a concentration of the transgene product at a Cmin of at least 0.330 μg/mL in the eye (e.g., Vitreous humor), or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Transgene concentrations can be measured directly in patient samples of fluid collected from a bodily fluid, ocular fluid, vitreous humor, or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml (GC/mL) or the number of genome copies administered to the eye of the patient (e.g., administered suprachoroidally). In some embodiments, 2.4×10¹¹ GC/mL to 1×10¹³ GC/mL are administered, 2.4×10¹¹ GC/mL to 5×10¹¹ GC/mL are administered, 5×10¹¹ GC/mL to 1×10¹² GC/mL are administered, 1×10¹² GC/mL to 5×10¹² GC/mL are administered, or 5×10¹² GC/mL to 1×10¹³ GC/mL are administered. In some embodiments, 1.5×10¹³ GC/mL to 3×10¹³ GC/mL are administered. In some embodiments, about 2.4×10¹¹ GC/mL, about 5×10¹¹ GC/mL, about 1×10¹² GC/mL, about 2.5×10¹² GC/mL, about 5×10¹² GC/mL, about 1×10¹³ GC/mL or about 1.5×10¹³ GC/mL are administered. In some embodiments, 1×10⁹ to 1×10¹² genome copies are administered. In some embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ GC/mL in a volume of 250 μl). In another specific embodiment, about 1×10¹⁰ genome copies are administered (which corresponds to about 4×10¹⁰ GC/mL in a volume of 250 μl). In another specific embodiment, about 6×10¹⁰ genome copies are administered (which corresponds to about 2.4×10¹¹ GC/mL in a volume of 250 μl). In another specific embodiment, about 6.4×10¹⁰ genome copies are administered (which corresponds to about 3.2×10¹¹ GC/mL in a volume of 200 μl). In another specific embodiment, about 1.3×10¹¹ genome copies are administered (which corresponds to about 6.5×10¹¹ GC/mL in a volume of 200 μl). In another specific embodiment, about 2.5×10¹¹ genome copies are administered (which corresponds to about 2.5×10¹² GC/mL in a volume of 100 μl). In another specific embodiment, about 5×10¹¹ genome copies are administered (which corresponds to about 5×10¹² GC/mL in a volume of 200 μl). In another specific embodiment, about 1.5×10¹² genome copies are administered (which corresponds to about 1.5×10¹³ GC/mL in a volume of 100 μl). In some embodiments, about 6.4×10¹⁰ genome copies are administered per eye, or per dose, or per route of administration. In some embodiments, about 6.4×10¹⁰ genome copies is the total number of genome copies administered. In some embodiments, about 1.3×10¹¹ genome copies are administered per eye, or per dose, or per route of administration. In some embodiments, about 1.3×10¹¹ genome copies is the total number of genome copies administered. In some embodiments, about 2.5×10¹¹ genome copies are administered per eye, or per dose, or per route of administration. In some embodiments, about 2.5×10¹¹ genome copies is the total number of genome copies administered. In some embodiments, about 5×10¹¹ genome copies are administered per eye, or per dose, or per route of administration. In some embodiments, about 5×10¹¹ genome copies is the total number of genome copies administered. In some embodiments, about 1.5×10¹² genome copies are administered per eye, or per dose, or per route of administration. In some embodiments, about 1.5×10¹² genome copies is the total number of genome copies administered. In some embodiments, about 3×10¹² genome copies are administered per eye, or per dose, or per route of administration. In some embodiments, about 3×10¹² genome copies is the total number of genome copies administered. In another specific embodiment, about 1.6×10¹¹ genome copies are administered (which corresponds to about 6.2×10¹¹ GC/mL in a volume of 250 μl). In another specific embodiment, about 1.55×10¹¹ genome copies are administered (which corresponds to about 6.2×10¹¹ GC/mL in a volume of 250 μl). In another specific embodiment, about 1.6×10¹¹ genome copies are administered (which corresponds to about 6.4×10¹¹ GC/mL in a volume of 250 μl). In another specific embodiment, about 2.5×10¹¹ genome copies (which corresponds to about 1.0×10¹² in a volume of 250 μl) are administered. In another specific embodiment, about 3×10¹¹ genome copies are administered (which corresponds to about 3×10¹² GC/mL in a volume of 100 μl). In another specific embodiment, about 6×10¹¹ genome copies are administered (which corresponds to about 3×10¹² GC/mL in a volume of 200 μl). In another specific embodiment, about 6×10¹¹ genome copies are administered (which corresponds to about 6×10¹² GC/mL in a volume of 100 μl).

In certain embodiments, about 6.0×10¹⁰ genome copies per administration, or per eye are administered. In certain embodiments, about 6.4×10¹⁰ genome copies per administration, or per eye are administered. In certain embodiments, about 1.3×10¹¹ genome copies per administration, or per eye are administered. In certain embodiments, about 1.5×10¹¹ genome copies per administration, or per eye are administered. In certain embodiments, about 1.6×10¹¹ genome copies per administration, or per eye are administered. In certain embodiments, about 2.5×10¹¹ genome copies per administration, or per eye are administered. In certain embodiments, about 3×10¹¹ genome copies per administration, or per eye are administered. In certain embodiments, about 5.0×10¹¹ genome copies per administration, or per eye are administered. In certain embodiments, about 6×10¹¹ genome copies per administration, or per eye are administered. In certain embodiments, about 3×10¹² genome copies per administration, or per eye are administered. In certain embodiments, about 1.0×10¹² GC/mL per administration, or per eye are administered. In certain embodiments, about 2.5×10¹² GC/mL per administration, or per eye are administered. In certain embodiments, about 3×10¹² GC/mL per administration, or per eye are administered. In certain embodiments, about 3.0×10¹³ genome copies per administration, or per eye are administered. In certain embodiments, up to 3.0×10¹³ genome copies per administration, or per eye are administered.

In certain embodiments, about 1.5×10¹¹ genome copies per administration, or per eye are administered by suprachoroidal injection. In certain embodiments, about 2.5×10¹¹ genome copies per administration, or per eye are administered by suprachoroidal injection. In certain embodiments, about 3×10¹¹ genome copies per administration, or per eye are administered by suprachoroidal injection. In certain embodiments, about 5.0×10¹¹ genome copies per administration, or per eye are administered by suprachoroidal injection. In certain embodiments, about 6×10¹¹ genome copies per administration, or per eye are administered by suprachoroidal injection. In certain embodiments, about 1.5×10¹² genome copies per administration, or per eye are administered by suprachoroidal injection. In certain embodiments, about 3×10¹² genome copies per administration, or per eye are administered by suprachoroidal injection. In certain embodiments, about 2.5×10¹¹ genome copies per eye are administered by a single suprachoroidal injection. In certain embodiments, about 3×10¹¹ genome copies per administration, or per eye are administered by a single suprachoroidal injection. In certain embodiments, about 3×10¹¹ genome copies per administration, or per eye are administered by a single suprachoroidal injection in a volume of 100 μl. In certain embodiments, about 3×10¹¹ genome copies per administration, or per eye are administered by a single suprachoroidal injection in a volume of 200 μl. In certain embodiments, about 3×10¹¹ genome copies per administration, or per eye are administered by double suprachoroidal injections. In certain embodiments, about 3×10¹¹ genome copies per administration, or per eye are administered by double suprachoroidal injections, wherein each injection is in a volume of 50 μl. In certain embodiments, about 3×10¹¹ genome copies per administration, or per eye are administered by double suprachoroidal injections, wherein each injection is in a volume of 100 μl. In certain embodiments, about 5.0×10¹¹ genome copies per administration, or per eye are administered by double suprachoroidal injections. In certain embodiments, about 6×10¹¹ genome copies per administration, or per eye are administered by a single suprachoroidal injection. In certain embodiments, about 6×10¹¹ genome copies per administration, or per eye are administered by a single suprachoroidal injection in a volume of 100 μl. In certain embodiments, about 6×10¹¹ genome copies per administration, or per eye are administered by a single suprachoroidal injection in a volume of 200 μl. In certain embodiments, about 6×10¹¹ genome copies per administration, or per eye are administered by double suprachoroidal injections. In certain embodiments, about 6×10¹¹ genome copies per administration, or per eye are administered by double suprachoroidal injections, wherein each injection is in a volume of 50 μl. In certain embodiments, about 6×10¹¹ genome copies per administration, or per eye are administered by double suprachoroidal injections, wherein each injection is in a volume of 100 μl. In certain embodiments, about 3.0×10¹³ genome copies per administration, or per eye are administered by suprachoroidal injection. In certain embodiments, up to 3.0×10¹³ genome copies per administration, or per eye are administered by suprachoroidal injection. In certain embodiments, about 2.5×10¹² GC/mL per eye are administered by a single suprachoroidal injection in a volume of 100 μl. In certain embodiments, about 2.5×10¹² GC/mL per eye are administered by double suprachoroidal injections, wherein each injection is in a volume of 100 μl. In certain embodiments, about 1.5×10¹³ GC/mL per eye are administered by a single suprachoroidal injection in a volume of 100 μl.

In certain embodiments, the recombinant viral vector is administered by double suprachoroidal injections. In certain embodiments, the first injection in the right eye is administered in the superior temporal quadrant (i.e., between the 10 o'clock and 11 o'clock positions), and the second injection in the same eye is administered in the inferior nasal quadrant (i.e., between the 4 o'clock and 5 o'clock positions). In certain embodiments, the first injection in the right eye is administered in the inferior nasal quadrant (i.e., between the 4 o'clock and 5 o'clock positions), and the second injection in the same eye is administered in the superior temporal quadrant (i.e., between the 10 o'clock and 11 o'clock positions). In certain embodiments, the first injection in the left eye is administered in the superior temporal quadrant (i.e., between the 1 o'clock and 2 o'clock positions), and the second injection in the same eye is administered in the inferior nasal quadrant (i.e., between the 7 o'clock and 8 o'clock positions). In certain embodiments, the first injection in the left eye is administered in the inferior nasal quadrant (i.e., between the 7 o'clock and 8 o'clock positions), and the second injection in the same eye is administered in the superior temporal quadrant (i.e., between the 1 o'clock and 2 o'clock positions).

In certain embodiments, the recombinant viral vector is administered by a single suprachoroidal injection. In certain embodiments, the single injection in the right eye is administered in the superior temporal quadrant (i.e., between the 10 o'clock and 11 o'clock positions). In certain embodiments, the single injection in the right eye is administered in the inferior nasal quadrant (i.e., between the 4 o'clock and 5 o'clock positions). In certain embodiments, the single injection in the left eye is administered in the superior temporal quadrant (i.e., between the 1 o'clock and 2 o'clock positions). In certain embodiments, the single injection in the left eye is administered in the inferior nasal quadrant (i.e., between the 7 o'clock and 8 o'clock positions).

In some embodiments, the pharmaceutical composition or the reference pharmaceutical composition is administered to a human subject (e.g., suprachoroidally, subretinally, or intravitreously) once, twice, three times, four times, five times, six times, seven times, eight times, nine times, ten times, fifteen times, twenty times, twenty-five times, or thirty times. In some embodiments, the pharmaceutical composition or the reference pharmaceutical composition is administered to a human subject once in one day, twice in one day, three times in one day, four times in one day, five times in one day, six times in one day, or seven times in one day. In some embodiments, the same amount of AAV genome copies are administered per administration. For example, the same genome copies are administered suprachoroidally, subretinally, or intravitreously. In some embodiments, the same total amount of AAV genome copies are administered. For example, the same total amount of AAV genome copies are administered suprachoroidally, subretinally, or intravitreously regardless of the number of total administrations (e.g., if subretinal administration is performed once and suprachoroidal administration is performed twice, the genome copies in the one subretinal administration is the same as the genome copies in both suprachoroidal administrations combined).

As used herein and unless otherwise specified, the term “about” means within plus or minus 10% of a given value or range

### 4.4 Constructs and Formulations

In some embodiments, the recombinant vectors provided herein comprise the following elements in the following order: a) a constitutive or a hypoxia-inducible promoter sequence, and b) a sequence encoding the transgene (e.g., therapeutic product). In certain embodiments, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene, i) a second UTR sequence, j) a fourth linker sequence, k) a poly A sequence, l) a fifth linker sequence, and m) a second ITR sequence.

In certain embodiments, the recombinant vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene, i) a second UTR sequence, j) a fourth linker sequence, k) a poly A sequence, l) a fifth linker sequence, and m) a second ITR sequence, wherein the transgene encodes a light chain and a heavy chain sequence separated by a cleavable F/F2A sequence.

In some embodiments, the AAV (AAV viral vectors) provided herein comprise the following elements in the following order: a) a constitutive or a hypoxia-inducible promoter sequence, and b) a sequence encoding the transgene. In some embodiments, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In some embodiments, the viral vector comprises a signal peptide. In some embodiments, the signal peptide is MYRMQLLLLIALSLALVTNS (SEQ ID NO: 55). In some embodiments, the signal peptide is derived from IL-2 signal sequence. In some embodiments, the viral vector comprises a signal peptide from any signal peptide disclosed in Table 1, such as MNFLLSWVHW SLALLLYLHH AKWSQA (VEGF-A signal peptide) (SEQ ID NO: 5); MERAAPSRRV PLPLLLLGGL ALLAAGVDA (Fibulin-1 signal peptide) (SEQ ID NO: 6); MAPLRPLLIL ALLAWVALA (Vitronectin signal peptide) (SEQ ID NO: 7); MRLLAKIICLMLWAICVA (Complement Factor H signal peptide) (SEQ ID NO: 8); MRLLAFLSLL ALVLQETGT (Opticin signal peptide) (SEQ ID NO: 9); MKWVTFISLLFLFSSAYS (Albumin signal peptide) (SEQ ID NO: 22); MAFLWLLSCWALLGTTFG (Chymotrypsinogen signal peptide) (SEQ ID NO: 23); MYRMQLLSCIALILALVTNS (Interleukin-2 signal peptide) (SEQ ID NO: 24); MNLLLILTFVAAAVA (Trypsinogen-2 signal peptide) (SEQ ID NO: 25); or MYRMQLLLLIALSLALVTNS (mutant Interleukin-2 signal peptide) (SEQ ID NO: 55).

In some embodiments, the viral vector or other expression construct suitable for packaging in an AAV capsid, comprises (1) AAV inverted terminal repeats (ITRs) flank the expression cassette; (2) regulatory control elements, consisting essentially of one or more enhancers and/or promoters, d) a poly A signal, and e) optionally an intron; and (3) a transgene providing (e.g., coding for) one or more RNA or protein products of interest.

In some aspects, the disclosure provides for a nucleic acid for use, wherein the nucleic acid encodes a therapeutic product operatively linked to a promoter or enhancer-promoter described herein.

In some aspects, the disclosure provides for a nucleic acid for use, wherein the nucleic acid encodes a transgene operatively linked to a promoter selected from the group consisting of: the CB7 promoter (a chicken β-actin promoter and CMV enhancer), cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter. In a specific embodiment, the transgene is operatively linked to the CB7 promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence encoding the therapeutic product of interest (the transgene), untranslated regions, and termination sequences. In certain embodiments, recombinant vectors provided herein comprise a promoter operably linked to the sequence encoding the therapeutic product of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In certain embodiments, the recombinant vectors provided herein comprise modified mRNA encoding for the therapeutic product of interest (e.g., the transgene). In certain embodiments, the recombinant vectors provided herein comprise a nucleotide sequence encoding for a therapeutic product that is an shRNA, siRNA, or miRNA.

In certain embodiments, the vectors provided herein comprise components that modulate protein delivery. In certain embodiments, the viral vectors provided herein comprise one or more signal peptides. Examples of signal peptides include, but is not limited to, VEGF-A signal peptide (SEQ ID NO: 5), fibulin-1 signal peptide (SEQ ID NO: 6), vitronectin signal peptide (SEQ ID NO: 7), complement Factor H signal peptide (SEQ ID NO: 8), opticin signal peptide (SEQ ID NO: 9), albumin signal peptide (SEQ ID NO: 22), chymotrypsinogen signal peptide (SEQ ID NO: 23), interleukin-2 signal peptide (SEQ ID NO: 24), and trypsinogen-2 signal peptide (SEQ ID NO: 25), mutant interleukin-2 signal peptide (SEQ ID NO: 55).

### (a) Viral Vectors

In some embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHIM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In preferred embodiments, AAV based vectors provided herein comprise components from one or more of AAV8, AAV9, AAV10, AAV11, or AAVrh10 serotypes. In certain embodiments, the recombinant viral vectors provided herein are altered such that they are replication-deficient in humans. In certain embodiments, the recombinant viral vectors are hybrid vectors, e.g., an AAV vector placed into a “helpless” adenoviral vector. In certain embodiments, provided herein are recombinant viral vectors comprising a viral capsid from a first virus and viral envelope proteins from a second virus. In specific embodiments, the second virus is vesicular stomatitis virus (VSV). In more specific embodiments, the envelope protein is VSV-G protein.

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV.PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. Nos. 7,282,199 B2, 7,790,449 B2, 8,318,480 B2, 8,962,332 B2 and International Patent Application No. PCT/EP2014/076466, each of which is incorporated herein by reference in its entirety. In one aspect, provided herein are AAV (e.g., AAV8)-based viral vectors encoding a transgene.

In some embodiments, recombinant AAV viral vectors have been shown to have particular tropism for ocular tissues and are efficient at transducing and expressing the transgene product of such recombinant AAV. Thus, ocular-tropic vectors are useful in the methods and pharmaceutical compositions disclosed herein. In some embodiments, the method or pharmaceutical composition disclosed herein comprises an AAV viral vector having enhanced tropism for posterior segments of the eye, such as retina and RPE/choroid. In some embodiments, the method or pharmaceutical composition disclosed herein comprises a recombinant AAV3B viral vector. In certain embodiments, the AAV vector is an AAV vector disclosed in PCT International Application No. PCT/US2021/054008 (PCT International Publication No. WO2022076711A2, published Apr. 14, 2022), which is incorporated herein by reference in its entirety.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In a specific embodiment, a vector for use in the methods described herein is one that encodes transgene such that, upon introduction of the vector into a relevant cell (e.g., a retinal cell in vivo or in vitro), a glycosylated and or tyrosine sulfated variant of the transgene product is expressed by the cell. In a specific embodiment, the expressed transgene product comprises a glycosylation and/or tyrosine sulfation pattern.

### (b) Therapeutic Product or Transgenes

The therapeutic products can be, for example, therapeutic proteins (for example, antibodies), therapeutic RNAs (for example, shRNAs, siRNAs, and miRNAs), or therapeutic aptamers.

In certain embodiments, the disclosure provides a pharmaceutical composition comprising recombinant AAV encoding a transgene. In some embodiments, provided herein are rAAV viral vectors which do not encode an anti-VEGF Fab or anti-VEGF antibody. In some embodiments, provided herein are rAAV8-based viral vectors which do not encode an anti-VEGF Fab or anti-VEGF antibody. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV viral vectors encoding anti-kallikrein (anti-pKal) protein. In some embodiments, provided herein are rAAV8-based or rAAV9-based viral vectors encoding lanadelumab Fab or full-length antibody. In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain embodiments, provided herein are rAAV viral vectors encoding etanercept (an anti-TNF fusion protein). In certain embodiments, provided herein are rAAV viral vectors encoding adalimumab antibody (an anti-TNF antibody) or an antigen-binding fragment thereof. In certain embodiments, provided herein are rAAV viral vectors encoding a full-length anti-C3 or anti-C5 antibody, an anti-C3 or anti-C5 Fab, a complement factor H (CFH), or a complement factor H-like (CFHL-1) protein. In certain embodiments, provided herein are rAAV viral vectors encoding eculizumab, ravulizumab, tesidolumab, crovalimab, NGM621 or BB5.1 antibody, or an antigen-binding fragment thereof.

In certain embodiments, the therapeutic product (e.g., transgene) is: (1) Palmitoyl-Protein Thioesterase 1 (PPT1); (2) Tripeptidyl-Peptidase 1 (TPP1); (3) Battenin (CLN3); and (4) CLN6 Transmembrane ER Protein (CLN6).

In certain embodiments, the disclosure provides a pharmaceutical composition comprising recombinant AAV encoding a transgene. In some embodiments, provided herein are rAAV viral vectors encoding tripeptidyl peptidase 1 (TPP1) protein. In some embodiments, provided herein are rAAV9-based viral vectors encoding TPPL. In some embodiments, provided herein are rAAV viral vectors encoding anti-kallikrein (anti-pKal) protein, such as lanadelumab. In some embodiments, provided herein are rAAV8-based or rAAV9-based viral vectors encoding lanadelumab Fab or full-length antibody. In certain embodiments, the rAAV vector is an AAV vector encoding an antibody transgene disclosed in PCT International Application No. PCT/US2020/029802 (PCT International Publication No. WO2020219868A1, published Oct. 29, 2020), which is incorporated herein by reference in its entirety.

In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain embodiments, the vectors provided herein can be used for (1) the pathology of the eye associated with Batten-CLN1 and the therapeutic product is Palmitoyl-Protein Thioesterase 1 (PPT1); (2) the pathology of the eye associated with Batten-CLN2 and the therapeutic product is Tripeptidyl-Peptidase 1 (TPP1); (3) the pathology of the eye associated with Batten-CLN3 and the therapeutic product is Battenin (CLN3); (4) the pathology of the eye associated with Batten-CLN6 and the therapeutic product is CLN6 Transmembrane ER Protein (CLN6); (5) the pathology of the eye associated with Batten-CLN7 and the therapeutic product is Major Facilitator Superfamily Domain Containing 8 (MVFSD8); and (6) the pathology of the eye associated with Batten-CLN1 and the therapeutic product is Palmitoyl-Protein Thioesterase 1 (PPT1).

TABLE 1
Exemplary sequences

| SEQ ID | | |
|---|---|---|
| NO: | Description | Sequence |
| 5 | VEGF-A signal | MNFLLSWVHW SLALLLYLHH AKWSQA |
| | peptide | |
| 6 | Fibulin-1 signal | MERAAPSRRV PLPLLLLGGL ALLAAGVDA |
| | peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 22 | Albumin signal | MKWVTFISLLFLFSSAYS |
| | peptide | |
| 23 | Chymotrypsinog | MAFLWLLSCWALLGTTFG |
| | en signal peptide | |
| 24 | Interleukin-2 | MYRMQLLSCIALILALVTNS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG)EGRGSLLTCGDVEENPGP |
| 28 | P2A site | (GSG)ATNFSLLKQAGDVEENPGP |
| 29 | E2A site | (GSG)QCTNYALLKLAGDVESNPGP |
| 30 | F2A site | (GSG)VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 41 | AAV1 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEEVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDEDKFFPMSGVMIFGKESAGA |
| | | SNTALDNVMITDEEEIKATNPVATERFGTVAVNFQSSSTDPATGDVHAMGALPGM |
| | | VWQDRDVYLQGPIWAKIPHTDGHFHPSPLMGGFGLKNPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 42 | AAV2 | MAADGYLPDWLEDTLSEGIRQWWKLKPGPPPPKPAERHKDDSRGLVLPGYKYLGP |
| | | FNGLDKGEPVNEADAAALEHDKAYDRQLDSGDNPYLKYNHADAEFQERLKEDTSF |
| | | GGNLGRAVFQAKKRVLEPLGLVEEPVKTAPGKKRPVEHSPVEPDSSSGTGKAGQQ |
| | | PARKRLNFGQTGDADSVPDPQPLGQPPAAPSGLGTNTMATGSGAPMADNNEGADG |
| | | VGNSSGNWHCDSTWMGDRVITTSTRTWALPTYNNHLYKQISSQSGASNDNHYFGY |
| | | STPWGYFDFNRFHCHFSPRDWQRLINNNWGFRPKRLNFKLFNIQVKEVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADN |
| | | NNSEYSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGVLPGMV |
| | | WQDRDVYLQGPIWAKIPHTDGHFHPSPLMGGFGLKHPPPQILIKNTPVPANPSTT |
| | | FSAAKFASFITQYSTGQVSVEIEWELQKENSKRWNPEIQYTSNYNKSVNVDFTVD |
| | | TNGVYSEPRPIGTRYLTRNL |
| 43 | AAV3-3 | MAADGYLPDWLEDNLSEGIREWWALKPGVPQPKANQQHQDNRRGLVLPGYKYLGP |
| | | GNGLDKGEPVNEADAAALEHDKAYDQQLKAGDNPYLKYNHADAEFQERLQEDTSF |
| | | GGNLGRAVFQAKKRILEPLGLVEEAAKTAPGKKGAVDQSPQEPDSSSGVGKSGKQ |
| | | PARKRLNFGQTGDSESVPDPQPLGEPPAAPTSLGSNTMASGGGAPMADNNEGADG |
| | | VGNSSGNWHCDSQWLGDRVITTSTRTWALPTYNNHLYKQISSQSGASNDNHYFGY |
| | | STPWGYFDFNRFHCHFSPRDWQRLINNNWGFRPKKLSFKLFNIQVRGVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFQFSYTFEDVPFHSSYAHSQSLDRLMNPLIDQ |
| | | YLYYLNRTQGTTSGTTNQSRLLFSQAGPQSMSLQARNWLPGPCYRQQRLSKTAND |
| | | NNNSNFPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTGTVNHQGALPGM |
| | | VWQDRDVYLQGPIWAKIPHTDGHFHPSPLMGGFGLKHPPPQIMIKNTPVPANPPT |
| | | TFSPAKFASFITQYSTGQVSVEIEWELQKENSKRWNPEIQYTSNYNKSVNVDFTV |
| | | DTNGVYSEPRPIGTRYLTRNL |
| 44 | AAV4-4 | MTDGYLPDWLEDNLSEGVREWWALQPGAPKPKANQQHQDNARGLVLPGYKYLGPG |
| | | NGLDKGEPVNAADAAALEHDKAYDQQLKAGDNPYLKYNHADAEFQQRLQGDTSFG |
| | | GNLGRAVFQAKKRVLEPLGLVEQAGETAPGKKRPLIESPQQPDSSTGIGKKGKQP |
| | | AKKKLVFEDETGAGDGPPEGSTSGAMSDDSEMRAAAGGAAVEGGQGADGVGNASG |
| | | DWHCDSTWSEGHVTTTSTRTWVLPTYNNHLYKRLGESLQSNTYNGFSTPWGYFDF |
| | | NRFHCHFSPRDWQRLINNNWGMRPKAMRVKIFNIQVKEVTTSNGETTVANNLTST |
| | | VQIFADSSYELPYVMDAGQEGSLPPFPNDVFMVPQYGYCGLVTGNTSQQQTDRNA |
| | | FYCLEYFPSQMLRTGNNFEITYSFEKVPFHSMYAHSQSLDRLMNPLIDQYLWGLQ |
| | | STTTGTTLNAGTATTNFTKLRPTNFSNFKKNWLPGPSIKQQGFSKTANQNYKIPA |
| | | TGSDSLIKYETHSTLDGRWSALTPGPPMATAGPADSKFSNSQLIFAGPKQNGNTA |
| | | TVPGTLIFTSEEELAATNATDTDMWGNLPGGDQSNSNLPTVDRLTALGAVPGMVW |
| | | QNRDIYYQGPIWAKIPHTDGHFHPSPLIGGFGLKHPPPQIFIKNTPVPANPATTF |
| | | SSTPVNSFITQYSTGQVSVQIDWEIQKERSKRWNPEVQFTSNYGQQNSLLWAPDA |
| | | AGKYTEPRAIGTRYLTHHL |
| 45 | AAV5 | MSFVDHPPDWLEEVGEGLREFLGLEAGPPKPKPNQQHQDQARGLVLPGYNYLGPG |
| | | NGLDRGEPVNRADEVAREHDISYNEQLEAGDNPYLKYNHADAEFQEKLADDTSFG |
| | | GNLGKAVFQAKKRVLEPFGLVEEGAKTAPTGKRIDDHFPKRKKARTEEDSKPSTS |
| | | SDAEAGPSGSQQLQIPAQPASSLGADTMSAGGGGPLGDNNQGADGVGNASGDWHC |
| | | DSTWMGDRVVTKSTRTWVLPSYNNHQYREIKSGSVDGSNANAYFGYSTPWGYFDF |
| | | NRFHSHWSPRDWQRLINNYWGFRPRSLRVKIFNIQVKEVTVQDSTTTIANNLTST |
| | | VQVFTDDDYQLPYVVGNGTEGCLPAFPPQVFTLPQYGYATLNRDNTENPTERSSF |
| | | FCLEYFPSKMLRTGNNFEFTYNFEEVPFHSSFAPSQNLFKLANPLVDQYLYRFVS |
| | | TNNTGGVQFNKNLAGRYANTYKNWFPGPMGRTQGWNLGSGVNRASVSAFATTNRM |
| | | ELEGASYQVPPQPNGMTNNLQGSNTYALENTMIFNSQPANPGTTATYLEGNMLIT |
| | | SESETQPVNRVAYNVGGQMATNNQSSTTAPATGTYNLQEIVPGSVWMERDVYLQG |
| | | PIWAKIPETGAHFHPSPAMGGFGLKHPPPMMLIKNTPVPGNITSFSDVPVSSFIT |
| | | QYSTGQVTVEMEWELKKENSKRWNPEIQYTNNYNDPQFVDFAPDSTGEYRTTRPI |
| | | GTRYLTRPL |
| 46 | AAV6 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPFGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMGALPGM |
| | | VWQDRDVYLQGPIWAKIPHTDGHFHPSPLMGGFGLKHPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 47 | AAV7 | MAADGYLPDWLEDNLSEGIREWWDLKPGAPKPKANQQKQDNGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPAKKRPVEPSPQRSPDSSTGIGKKGQ |
| | | QPARKRLNFGQTGDSESVPDPQPLGEPPAAPSSVGSGTVAAGGGAPMADNNEGAD |
| | | GVGNASGNWHCDSTWLGDRVITTSTRTWALPTYNNHLYKQISSETAGSTNDNTYF |
| | | GYSTPWGYFDFNRFHCHFSPRDWQRLINNNWGFRPKKLRFKLFNIQVKEVTTNDG |
| | | VTTIANNLTSTIQVFSDSEYQLPYVLGSAHQGCLPPFPADVFMIPQYGYLTLNNG |
| | | SQSVGRSSFYCLEYFPSQMLRTGNNFEFSYSFEDVPFHSSYAHSQSLDRLMNPLI |
| | | DQYLYYLARTQSNPGGTAGNRELQFYQGGPSTMAEQAKNWLPGPCFRQQRVSKTL |
| | | DQNNNSNFAWTGATKYHLNGRNSLVNPGVAMATHKDDEDRFFPSSGVLIFGKTGA |
| | | TNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPP |
| | | EVFTPAKFASFITQYSTGQVSVEIEWELQKENSKRWNPEIQYTSNFEKQTGVDFA |
| | | VDSQGVYSEPRPIGTRYLTRNL |
| 48 | AAV8 | MAADGYLPDWLEDNLSEGIREWWALKPGAPKPKANQQKQDDGRGLVLPGYKYLGP |
| | | FNGLDKGEPVNAADAAALEHDKAYDQQLQAGDNPYLRYNHADAEFQERLQEDTSF |
| | | GGNLGRAVFQAKKRVLEPLGLVEEGAKTAPGKKRPVEPSPQRSPDSSTGIGKKGQ |
| | | QPARKRLNFGQTGDSESVPDPQPLGEPPAAPSGVGPNTMAAGGGAPMADNNEGAD |
| | | GVGSSSGNWHCDSTWLGDRVITTSTRTWALPTYNNHLYKQISNGTSGGATNDNTY |
| | | FGYSTPWGYFDFNRFHCHFSPRDWQRLINNNWGFRPKRLSFKLFNIQVKEVTQNE |
| | | GTKTIANNLTSTIQVFTDSEYQLPYVLGSAHQGCLPPFPADVFMIPQYGYLTLNN |
| | | GSQAVGRSSFYCLEYFPSQMLRTGNNFQFTYTFEDVPFHSSYAHSQSLDRLMNPL |
| | | IDQYLYYLSRTQTTGGTANTQTLGFSQGGPNTMANQAKNWLPGPCYRQQRVSTTT |
| | | GQNNNSNFAWTAGTKYHLNGRNSLANPGIAMATHKDDEERFFPSNGILIFGKQNA |
| | | ARDNADYSDVMLTSEEEIKTTNPVATEEYGIVADNLQQQNTAPQIGTVNSQGALP |
| | | GMVWQNRDVYLQGPIWAKIPHTDGNFHPSPLMGGFGLKHPPPQILIKNTPVPADP |
| | | PTTFNQSKLNSFITQYSTGQVSVEIEWELQKENSKRWNPEIQYTSNYYKSTSVDF |
| | | AVNTEGVYSEPRPIGTRYLTRNL |
| 49 | hu31 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | GQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | STEGVYSEPRPIGTRYLTRNL |
| 50 | hu32 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 51 | AAV9 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 53 | Palmitoyl-protein | MASPGCLWLLAVALLPWTCASRALQHLDPPAPLPLVIWHGMGDSCCNPLSMGAIK |
| | thioesterase 1 | KMVEKKIPGIYVLSLEIGKTLMEDVENSFFLNVNSQVTTVCQALAKDPKLQQGYN |
| | (ppt1) | AMGFSQGGQFLRAVAQRCPSPPMINLISVGGQHQGVFGLPRCPGESSHICDFIRK |
| | Aah08426.1 | TLNAGAYSKVVQERLVQAEYWHDPIKEDVYRNHSIFLADINQERGINESYKKNLM |
| | | ALKKFVMVKFLNDSIVDPVDSEWFGFYRSGQAKETIPLQETSLYTQDRLGLKEMD |
| | | NAGQLVFLATEGDHLQLSEEWFYAHIIPFLG |
| 54 | Tripeptidyl- | MGLQACLLGLFALILSGKCSYSPEPDQRRTLPPGWVSLGRADPEEELSLTFALRQ |
| | peptidase 1 | QNVERLSELVQAVSDPSSPQYGKYLTLENVADLVRPSPLTLHTVQKWLLAAGAQK |
| | (tpp1) | CHSVITQDFLTCWLSIRQAELLLPGAEFHHYVGGPTETHVVRSPHPYQLPQALAP |
| | Np_000382.3 | HVDFVGGLHRFPPTSSLRQRPEPQVTGTVGLHLGVTPSVIRKRYNLTSQDVGSGT |
| | | SNNSQACAQFLEQYFHDSDLAQFMRLFGGNFAHQASVARVVGQQGRGRAGIEASL |
| | | DVQYLMSAGANISTWVYSSPGRHEGQEPFLQWLMLLSNESALPHVHTVSYGDDED |
| | | SLSSAYIQRVNTELMKAAARGLTLLFASGDSGAGCWSVSGRHQFRPTFPASSPYV |
| | | TTVGGTSFQEPFLITNEIVDYISGGGFSNVFPRPSYQEEAVTKFLSSSPHLPPSS |
| | | YFNASGRAYPDVAALSDGYWVVSNRVPIPWVSGTSASTPVFGGILSLINEHRILS |
| | | GRPPLGFLNPRLYQQHGAGLFDVTRGCHESCLDEEVEGQGFCSGPGWDPVTGWGT |
| | | PNFPALLKTLLNP |
| 55 | Mutant | MYRMQLLLLIALSLALVINS |
| | interleukin-2 | |
| | signal peptide | |
| 56 | capsid protein | MAADGYLPDWLEDNLSEGIREWWALKPGVPQPKANQQHQDNRRGLVLPGYKYLGP |
| | VP1 for AAV3B | GNGLDKGEPVNEADAAALEHDKAYDQQLKAGDNPYLKYNHADAEFQERLQEDTSF |
| | | GGNLGRAVFQAKKRILEPLGLVEEAAKTAPGKKRPVDQSPQEPDSSSGVGKSGKQ |
| | | PARKRLNFGQTGDSESVPDPQPLGEPPAAPTSLGSNTMASGGGAPMADNNEGADG |
| | | VGNSSGNWHCDSQWLGDRVITTSTRTWALPTYNNHLYKQISSQSGASNDNHYFGY |
| | | STPWGYFDFNRFHCHFSPRDWQRLINNNWGFRPKKLSFKLFNIQVKEVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFQFSYTFEDVPFHSSYAHSQSLDRLMNPLIDQ |
| | | YLYYLNRTQGTTSGTTNQSRLLFSQAGPQSMSLQARNWLPGPCYRQQRLSKTAND |
| | | NNNSNFPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTRTVNDQGALPGM |
| | | VWQDRDVYLQGPIWAKIPHTDGHFHPSPLMGGFGLKHPPPQIMIKNTPVPANPPT |
| | | TFSPAKFASFITQYSTGQVSVEIEWELQKENSKRWNPEIQYTSNYNKSVNVDFTV |
| | | DTNGVYSEPRPIGTRYLTRNL |

### 4.5 Diseases

The pharmaceutical composition or the reference pharmaceutical composition provided herein (e.g., Section 4.1) can be administered to a subject diagnosed with nAMD (wet AMD), dry AMD, retinal vein occlusion (RVO), diabetic macular edema (DME), diabetic retinopathy (DR), or Batten disease.

In some embodiments, disclosed herein are methods of treating a subject diagnosed with nAMD (wet AMD), dry AMD, retinal vein occlusion (RVO), diabetic macular edema (DME), diabetic retinopathy (DR), or Batten by administering to the subject a therapeutically effective amount of the pharmaceutical composition by suprachoroidal injection (for example, via a suprachoroidal drug delivery device such as a microinjector with a microneedle).

In some embodiments, the pharmaceutical composition described herein is administered to a subject diagnosed with dry AMD, wherein the pharmaceutical composition comprises a rAAV vector encoding a transgene product that ameliorate disease pathology in the posterior segments of the eye. Such pharmaceutical composition can slow or arrest the progression of or relieve one or more symptoms of dry AMD, such as to reduce the rate of geographic atrophy or improve visual acuity (or reduce the rate of loss of visual acuity). The pharmaceutical composition can be administered suprachoroidally as a method to deliver transgene product to the retina and/or RPE-choroid or other posterior segments of the eye.

For dry AMD, physical changes to the eye, including changes in geographic atrophy can be measured Optical Coherence Tomography using methods known in the art. The compositions and methods described herein may be assessed for efficacy using in vitro complement inhibition assays, such as membrane attack complex (“MAC”) formation, C5a generation and hemolysis. Complement inhibition assays can be performed in any appropriate cell type, such as ARPE19 cells (MAC and C5a assays), iPSC-derived RPE cells (MAC and C5a assays) or sheep/rabbit erythrocytes (hemolysis assay). MAC formation assays measure the deposition of MAC on the surface of RPE cells (% relative inhibition of MAC formation). C5a generation assays measure the ability of the C5 antibody to prevent C5 cleavage (less C5 cleavage=less C5a). Hemolysis assays allow the comparison of complement inhibition among different complement inhibitors (50% complement inhibition dose (ng/ml) (CH50; AH50). Animal models may be administered vectors described herein, for example, suprachoroidally, and then assessed for geographic atrophy (or change therein) by OCT, retinal pathology (damage or repair to RPE), and other assessments of dry AMD pathology, as well as reduction in C3a or C5a, cleavage of C3 or C5 or other markers of complement activation.

Animals and animal models can be administered the pharmaceutical compositions described herein, for example, suprachoroidally, and then assessed for concentration of the transgene product in target tissues (see e.g., Examples 11-15 and 17).

In some embodiments, a pharmaceutical composition containing about 2.5×10¹¹ GC/eye, about 5×10¹¹ GC/eye, or about 1.5×10¹² GC/eye of a Construct (e.g., a construct of the disclosure) of a pharmaceutical composition comprising 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL, 4% w/v sucrose, and optionally a surfactant is administered to a patient via suprachoroidal administration. In some embodiments, the patient has diabetic retinopathy. In some embodiments, the patient has dry AMD.

In some embodiments, a pharmaceutical composition containing about 2.5×10¹¹ GC/eye, about 5×10¹¹ GC/eye, or about 1.5×10¹² GC/eye of a Construct of a pharmaceutical composition comprising 10% w/v sucrose is administered to a patient via suprachoroidal administration. In some embodiments, the patient has diabetic retinopathy. In some embodiments, the patient has dry AMD. In some embodiments, the pharmaceutical composition has a tonicity/osmolality equal to or greater than 240 mOsm/kg.

In some aspects, disclosed herein are pharmaceutical compositions suitable for treating a subject diagnosed with kallikrein-related disease. In some aspects, disclosed herein are methods for treating a subject diagnosed with kallikrein-related disease comprising administering to the subject a therapeutically effective amount of the pharmaceutical composition. In some embodiments, the pharmaceutical composition is administered in the SCS.

In some embodiments, the pharmaceutical composition or the reference pharmaceutical composition provided herein (e.g., Section 4.1) can be administered to a subject diagnosed with (1) Batten-CLN2 and the therapeutic product is Tripeptidyl-Peptidase 1 (TPP1); (2) Usher's-Type 1 and the therapeutic product is Myosin VIIA (MYO7A); (3) Usher's-Type 1 and the therapeutic product is Cadherin Related 23 (CDH23); (4) Usher's-Type 2 and the therapeutic product is Protocadherin Related 15 (PCDH15); (5) Usher's-Type 2 and the therapeutic product is Usherin (USH2A); (6) Usher's-Type 3 and the therapeutic product is Clarin 1 (CLRN1); (7) Stargardt's and the therapeutic product is ATP Binding Cassette Subfamily A Member 4 (ABCA4); (8) Stargardt's and the therapeutic product is ELOVL Fatty Acid Elongase 4 (ELOVL4); (9) red-green color blindness and the therapeutic product is L opsin (OPN1LW); (10) red-green color blindness and the therapeutic product is M opsin (OPN1MW); (11) blue cone monochromacy and the therapeutic product is M opsin (OPN1MW); (12) Leber congenital amaurosis-1 (LCA 1) and the therapeutic product is Guanylate Cyclase 2D, Retinal (GUCY2D); (13) Leber congenital amaurosis-2 (LCA 2) and the therapeutic product is Retinoid Isomerohydrolase RPE65 (RPE65); (14) Leber congenital amaurosis-4 (LCA 4) and the therapeutic product is Aryl Hydrocarbon Receptor Interacting Protein Like 1 (AIPL1); (15) Leber congenital amaurosis-7 (LCA 7) and the therapeutic product is Cone-Rod Homeobox (CRX); (16) Leber congenital amaurosis-8 (LCA 8) and the therapeutic product is Crumbs Cell Polarity Complex Component 1 (CRB1); (17) Leber congenital amaurosis-9 (LCA 9) and the therapeutic product is Nicotinamide Nucleotide Adenylyltransferase 1 (NMNAT1); (18) Leber congenital amaurosis-10 (LCA 10) and the therapeutic product is Centrosomal Protein 290 (CEP290); (19) Leber congenital amaurosis-11 (LCA 11) and the therapeutic product is Inosine Monophosphate Dehydrogenase 1 (IMPDH1); (20) Leber congenital amaurosis-15 (LCA 15) and the therapeutic product is Tubby Like Protein 1 (TULP1); (21) LHON and the therapeutic product is Mitochondrially Encoded NADH Dehydrogenase 4 (MT-ND4); (22) LHON and the therapeutic product is Mitochondrially Encoded NADH Dehydrogenase 6 (MT-ND6); (23) choroideremia and the therapeutic product is Rab Escort Protein 1 (CHM); (24) X-linked retinoschisis (XLRS) and the therapeutic product is Retinoschisin (RS1); (25) Bardet-Biedl syndrome 1 and the therapeutic product is Bardet-Biedl Syndrome 1 (BBS1); (26) Bardet-Biedl syndrome 6 and the therapeutic product is McKusick-Kaufman Syndrome (MKKS); (27) Bardet-Biedl syndrome 10 and the therapeutic product is Bardet-Biedl Syndrome 10 (BBS10); (28) cone dystrophy and the therapeutic product is Guanylate Cyclase Activator 1A (GUCA1A); (29) optic atrophy and the therapeutic product is OPA1 Mitochondrial Dynamin Like GTPase (OPA1); (30) retinitis pigmentosa 1 and the therapeutic product is RP1 Axonemal Microtubule Associated (RP1); (31) retinitis pigmentosa 2 and the therapeutic product is RP2 Activator of ARL3 GTPase (RP2); (32) retinitis pigmentosa 7 and the therapeutic product is Peripherin 2 (PRPH2); (33) retinitis pigmentosa 11 and the therapeutic product is Pre-mRNA Processing Factor 31 (PRPF31); (34) retinitis pigmentosa 13 and the therapeutic product is Pre-mRNA Processing Factor 8 (PRPF8); (35) retinitis pigmentosa 37 and the therapeutic product is Nuclear Receptor Subfamily 2 Group E Member 3 (NR2E3); (36) retinitis pigmentosa 38 and the therapeutic product is MER Proto-Oncogene, Tyrosine Kinase (MERTK); (37) retinitis pigmentosa 40 and the therapeutic product is Phosphodiesterase 6B (PDE6B); (38) retinitis pigmentosa 41 and the therapeutic product is Prominin 1 (PROM1); (39) retinitis pigmentosa 56 and the therapeutic product is Interphotoreceptor Matrix Proteoglycan 2 (IMPG2); (40) petinitis pigmentosa 62 and the therapeutic product is Male Germ Cell Associated Kinase (MAK); (41) retinitis pigmentosa 80 and the therapeutic product is Intraflagellar Transport 140 (IFT140); or (42) Best disease and the therapeutic product is Bestrophin 1 (BEST1).

### 4.6 Assays

The skilled artesian may use the assays as described herein and/or techniques known in the art to study the composition and methods described herein, for example to test the formulations provided herein. In some embodiments, a skilled person may use any assay and/or techniques described in, for example, Chiang et a. “Clearance Kinetics and Clearance Routes of Molecules From the Suprachoroidal Space After Microneedle Injection”, Invest Ophthalmol Vis Sci. 2017 January; 58(1): 545-554; Gu et al. “Real-Time Monitoring of Suprachoroidal Space (SCS) Following SCS Injection Using Ultra-High Resolution”, Optical Coherence Tomography in Guinea Pig Eyes”, Invest Ophthalmol Vis Sci. 2015 June; 56(6):3623-34; and Seiler et al. “Effect and Distribution of Contrast Medium after Injection into the Anterior Suprachoroidal Space in Ex Vivo Eyes”, Invest Ophthalmol Vis Sci. 2011 Jul. 29; 52(8):5730-6 (each of which is incorporated herein in its entirety), As detailed in Section 5, the following assays are also provided herein.

### 4.6.1 Ultrasound B-Scan

A high-frequency ultrasound (U/S) probe (UBM Plus; Accutome, Malvern, PA. USA) can be used to determine SCS thickness by generating 21) cross-sectional images of the SCS in animal eyes ex vivo after injecting different volumes ranging in levels of AAV aggregation. Different amounts of AAV aggregation can be injected. An U/S probe cover (Clearscan, Eye-Surgical-Instruments, Plymouth, MN) can be attached to the UBM Plus to facilitate U/S image acquisition. The U/S probe can be used to acquire sagittal views around the eye (e.g., eight sagittal views). Postprocessing of the U/S B-scans can be performed to find the thickness from the outer sclera to the inner retina at, for example, 1, 5, and 9 mm posterior to the scleral spur. The mean, median, and standard deviation for each eye can be calculated

### 4.6.2 SCS Distension

High-frequency (50 MHz) ultrasound (E-technologies, Bettendorf, IA) can be used to image the effect and distension of the injections on the SCS in real time. The ultrasound probe can be positioned immediately over the injection site so that the adjacent sclera, SCS, and ciliary body/choroid can be imaged in real time during the injection. Images can be collected, and the maximal distance that the SCS is distended when injected with PBS or the pharmaceutical composition can be measured with internal calipers of the ultrasound.

This technique can also be used to determine the effect of physiologic intraocular pressure (IOP) on SCS distension after injection and to determine the changes of IOP after SCS injection. After placement of the catheter in the SCS, a needle (e.g., 27-gauge) can be placed through the limbus into the anterior chamber, and cyano-acrylate tissue adhesive can be used to seal the limbal incision. The needle can be connected with 0.9% saline-filled tubing to a pressure transducer (MedEx LogiCal Transducer, Model MX960; MedExSupply Medical Supplies, Monsey, NY) and an electronic monitor, allowing for continuous measurement of IOP. Maximal distension of the SCS can be measured and reported using internal calipers of the ultrasound. The IOP (in mm Hg) at the time of maximal SCS distension can be recorded for each injection volume.

### 4.6.3 Two-Dimensional Ultrasound Contrast Imaging of SCS

Using contrast-enhanced ultrasound (Mylab70; Biosound Esaote, Inc., Indianapolis, IN), microbubble ultrasound contrast agent (Targestar-P, Targeson Inc., San Diego, CA) can be injected into the anterior suprachoroidal space through cannulas placed. Percentage of maximal distribution in the SCS of contrast agent can be determined in the sagittal ultrasound plane using image analysis software (Elements 4.0 [Adobe Photoshop]; ImageJ 1.42q). Using contrast medium detection software (Qontrast; Biosound Esaote, Inc., Indianapolis, IN), regions of interest can be placed over the entire SCS and the posterior SCS, and contrast enhancement over time can be measured as mean pixel intensity.

### 4.6.4 Three-Dimensional Ultrasound Contrast Imaging of SCS

Porcine ex vivo eyes can be imaged using a custom 3D contrast imaging system that interfaces a computer-controlled linear motion axis with a clinical ultrasound scanner (Acuson Sequoia; Siemens Medical Solutions, Malvern, PA). A 4-MHz (4-C1) transducer and contrast imaging (Cadence CPS; Siemens Medical Solutions, Malvern, PA) can be used. The porcine eye can be placed in a water bath, and microbubble contrast medium can be injected through catheters placed into the anterior SCS. Dynamic contrast medium inflow can be observed in a midsagittal plane, followed by 3D imaging of the entire globe to assess spatial distribution of the contrast agent. Percentage of maximal distribution in the SCS of contrast agent can be determined slice by slice using image analysis software (Elements 4.0 [Adobe Photoshop]; ImageJ 1.42q).

### 4.6.5 Measuring Aggregation

In some embodiments, a weighted average apparent diameter is measured by Dynamic Light Scattering (DLS) to determine aggregated AAV particles in the formulated solution. In some embodiments, percent aggregation can be measured by visual inspection of microscopic images.

### 4.6.6 Measuring SCS Thickness Based on Liquid Volume

3D cryo-reconstruction imaging can be used to measure SCS thickness. Animal eyes that are injected with, for example, 25 μL to 500 μL containing red-fluorescent particles are frozen a few minutes (e.g., 3-5 minutes) post injection and prepared for cryosectioning. Using a digital camera, one red-fluorescent image of the cryoblock of tissue can be obtained every 300 μm by slicing the sample with the cryostat. Image stacks consisting of red-fluorescence images are analyzed to determine SCS thickness.

### 4.6.7 Measuring SCS Thickness Based on Formulation

U/S B-scan can be used to determine SCS thickness after injection of pharmaceutical compositions ranging in the level of AAV aggregation into the SCS of animals. High-frequency ultrasound B-scan can be used to determine the rate of SCS collapse. Eight sagittal views over the pars plana can be acquired: (a) supranasal, over the injection site; (b) superior; (c) nasal; (d) supratemporal; (e) temporal; (f) infratemporal; (g) inferior; and (h) infranasal.

Off-line post processing can be performed on the U/S views to measure the SCS thickness. The U/S probe can have a minimum axial resolution of 15 μm. For each U/S view, a line segment 5 mm posterior to the scleral spur and perpendicular to the sclera can be created. A line can start at the outer surface of the sclera and end at the inner surface of the retina. The sclera and chorioretina can be included in the measurement to ensure the line is perpendicular. SCS thickness is then calculated by subtracting the tissue thickness from the measured line length. Curve fitting is done to determine the rate of SCS collapse.

U/S B-scan can be used to determine SCS thickness at multiple locations over time and the rate of SCS collapse can be calculated. The approximate clearance time of injected fluorescent material from the SCS can be found by taking fluorescence fundus images in the animal eyes in vivo over time (e.g., at various time points) until fluorescence is no longer detected.

### 4.6.8 SCS Clearance Kinetics by Fundus Imaging

To study the effect of AAV aggregation on movement in the SCS, different pharmaceutical compositions ranging in AAV aggregation levels and containing a fluorescein can be injected into the SCS. The approximate clearance rate or clearance time of injected fluorescent material from the SCS can be found by taking fluorescence fundus images over time in animal eyes in vivo. In some cases, the rate of clearance can be determined by determining the total clearance time and the clearance time constant (t_{clearance}) calculated using a curve fit derived from the normalized concentration of total fluorescent signal over time. Topical eye drops of tropicamide and phenylephrine (Akorn, Lake Forest, IL) can be administered prior to each imaging session to dilate the eye. A RetCam II (Clarity Medical Systems, Pleasanton, CA) with the 1300 lens attachment and the built-in fluorescein angiography module can be used to acquire the images. Multiple images can be taken with the blue light output from the RetCam II set at, for example, 0.0009, 1.6, and 2.4 W/m². In an attempt to capture the entire interior surface of the ocular globe, nine images can be captured: central, supranasal, superior, supratemporal, temporal, infratemporal, inferior, infranasal, and nasal. This allows imaging into the far periphery. Imaging can be done immediately after injection, at 1 h, every 3 h for 12 h, and every two days post-injection. The total clearance time, which can be defined as the first time point following injection in which fluorescence is not detectable by visual observation, is determined for all eyes injected. Fluorescein isothiocyanate-conjugated AAV (FITC-AAV), or FITC Conjugated-AAV capsid Protein-specific monoclonal antibody may be utilized in analogous experiments to track movement and clearance of AAV particles in the SCS. Methods for fluorescent labeling of AAV are known in the art (Shi, et al. Sci. Adv. 2020; 6: eaaz3621; and Tsui, T. Y., et al. Hepatology 42, 335-342 (2005). Antibodies (FITC Conjugated) recognizing many AAV serotypes are commercially available.

### 4.6.9 Flat Mount to Characterize 2D Circumferential Spread

Pharmaceutical compositions of the present disclosure containing fluorescein, or fluorescently labeled AAV, are injected into the SCS. After SCS injection and freezing, eyes can be prepared to assess the 2D spread of particles and fluorescein. The frozen eye are sliced open from the limbus to the posterior pole to generate equidistant scleral flaps. The resulting scleral flaps are splayed open and the frozen vitreous humor, lens, and aqueous humor are removed.

A digital SLR camera (Canon 60D, Canon, Melville, N.Y.) with a 100 mm lens (Canon) can be used to acquire brightfield and fluorescence images. Camera parameters are held constant. To acquire the area of fluorescein spread, a green optical band-pass filter (520±10 nm; Edmunds Optics, Barrington, N.J.) can be placed on the lens, and the sample can be illuminated by a lamp with the violet setting of a multicolor LED bulb (S Series RGB MR16/E26. HitLights, Baton Rouge, La.). To visualize the location of the red-fluorescent particles, a red filter (610±10 nm; Edmunds Optics) can be placed on the lens, and the sample can be illuminated with the same lamp switched to green light. The area of green and red fluorescence that are above threshold can be calculated for each eye using ImageJ (National Institutes of Health, Bethesda, Md.). Thresholding can be set manually based on visual inspection of background signal.

### 4.6.10 Intraocular Pressure Measurements

A pressure measurement system can be used to measure pressure in SCS after SCS injection. Animals can be terminally anesthetized by subcutaneous injection of a ketamine/xylazine cocktail. After SCS injection (N=4), pressure in the SCS can be measured every few minutes. Pressures are monitored until they reach their original baseline values from before injection (i.e., ˜15 mmHg). After the measurements, the animals are euthanized with a lethal dose of pentobarbital injected intravenously. A second set of SCS injections can be made in the animal postmortem. In postmortem measurements, pressure is only measured in the tissue space (i.e., SCS) where the injection was made.

In some cases, the intraocular pressure can be obtained by an ophthalmic tonometer (Tono-Pen; AVIA, Reichert Technologies, Depew, NY, USA) before and after the SCS injection. The intraocular pressure measurement can be stopped when the intraocular pressure is returned to the baseline level.

### 4.6.11 Temperature Stress Assay

A temperature stress development stability study can be conducted at 1.0×10¹² GC/mL over 4 days at 37° C. to evaluate the relative stability of formulations provided herein. Assays can be used to assess stability include but are not limited to in vitro relative potency (IVRP), vector genome concentration (VGC by ddPCR), free DNA by dye fluorescence, dynamic light scattering, appearance, and pH. Long-term development stability studies can be carried out for 12 months to demonstrate maintenance of in-vitro relative potency and other quality at −80° C. (≤−60° C.) and −20° C. (−25° C. to −15° C.) in the formulations provided herein.

### 4.6.12 In Vitro Relative Potency (IVRP) Assay

To relate the ddPCR GC titer to gene expression, an in vitro bioassay may be performed by transducing HEK293 cells and assaying the cell culture supernatant for transgene protein levels. HEK293 cells are plated onto three poly-D-lysine-coated 96-well tissue culture plates overnight. The cells are then pre-infected with wild-type human Ad5 virus followed by transduction with three independently prepared serial dilutions of AAV vector reference standard and test article, with each preparation plated onto separate plates at different positions. On the third day following transduction, the cell culture media is collected from the plates and measured for transgene protein levels via ELISA. For the ELISA, 96-well ELISA plates coated with receptor are blocked and then incubated with the collected cell culture media to capture transgene product produced by HEK293 cells. Fab-specific anti-human IgG antibody is used to detect the transgene protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

### 4.6.13 Vector Genome Concentration Assay

Vector genome concentration (GC) can also be evaluated using ddPCR. At various timepoints post injection, several mice are sacrificed, and ocular tissues are subjected to total DNA extraction and ddPCR assay for vector copy numbers. Copies of vector genome (transgene) per gram of tissue identified in various tissue sections at sequential timepoints reveals spread of AAV in the eye.

Total DNA from collected ocular tissue sections are extracted with the DNeasy Blood & Tissue Kit and the DNA concentration re measured using a Nanodrop spectrophotometer. To determine the vector copy numbers in the tissue sections, digital PCR was performed with Naica Crystal Digital PCR system (Stilla technologies). Two color multiplexing system were applied here to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types, such as RPE cells may reveal sustained delivery to the retina.

### 4.6.14 Free DNA Analysis Using Dye Fluorescence Assay

Free DNA can be determined by fluorescence of SYBR® Gold nucleic acid gel stain (‘SYBR Gold dye’) that is bound to DNA. The fluorescence can be measured using a microplate reader and quantitated with a DNA standard. The results in ng/μL can be reported.

Two approaches can be used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1E6 is a unit conversion factor:

Total DNA (ng/μL) estimated=1E6×GC/mL (OD)×M (g/mol)/6.02E23

In the second approach, the sample can be heated to 85° C. for 20 min with 0.05% poloxamer 188 and the actual DNA measured in the heated sample by the SYBR Gold dye assay can be used as the total. This therefore has the assumption that all the DNA was recovered and quantitated. For trending, either the raw ng/μL can be used or the percentage determined by a consistent method can be used.

### 4.6.15 Size Exclusion Chromatography (SEC)

SEC can be performed using a Sepax SRT SEC-1000 Peek column (PN 215950P-4630, SN: 8A11982, LN: BT090, 5 μm 1000A, 4.6×300 mm) on Waters Acquity Arc Equipment ID 0447 (C3PO), with a 25 mm pathlength flowcell. The mobile phase can be, for example, 20 mM sodium phosphate, 300 mM NaCl, 0.005% poloxamer 188, pH 6.5, with a flow rate of 0.35 mL/minute for 20 minutes, with the column at ambient temperature. Data collection can be performed with 2 point/second sampling rate and 1.2 nm resolution with 25 point mean smoothing at 214, 260, and 280 nm. The ideal target load can be 1.5×10¹¹ GC. The samples can be injected with 50 μL, about ⅓ of the ideal target or injected with 5 μL.

### 4.6.16 Dynamic Light Scattering (DLS) Assay

Dynamic light scattering (DLS) can be performed on a Wyatt DynaProIII using Corning 3540 384 well plates with a 30 μL sample volume. Ten acquisitions each for 10 s can be collected per replicate and there can be three replicate measurements per sample. The solvent can be set according to the solvent used in the samples, for example ‘PBS’ for an AAV vector in dPBS. Results not meeting data quality criteria (baseline, SOS, noise, fit) can be ‘marked’ and excluded from the analysis.

### 4.6.17 Viscosity Measurement

Viscosity can be measured using methods known in the art, for example methods provide in the United States Pharmacopeia (USP) published in 2019 and previous versions thereof (incorporated by reference herein in their entirety). Viscosity at low shear was measured using a capillary viscometer, using methods described in USP <911>.

Viscosity versus shear rate can be determined using a cone and plate rotational rheometer. Rheometry measurements are described in the United States Pharmacopeia (USP) USP <1911> and rotational viscometry is described in USP<912>. Rotational rheometry viscosity measurements can be collected with an AR-G2 rheometer equipped with a Peltier temperature control plate with a 60 mm 1° angle aluminum cone accessory (TA Instruments, New Castle, DE). A viscosity versus shear rate sweep can be performed over the range starting at <0.3 s-1 ramped up to 5000 s⁻¹ with 5 points per decade collected. The viscosity versus shear rate was collected at 20° C. Viscosity at 10,000 and 20,000 s⁻¹ were extrapolated from the data. In some cases, the viscosity of the pharmaceutical composition or the reference pharmaceutical composition can be measured at zero, 0.1 s-1, 1 s-1, 1000 s-1, 5000 s-1, 10,000 s-1, 20,000 s-1, or more than 20,000 s-1.

### 4.6.18 Virus Infectivity Assay

TCID₅₀ infectious titer assay as described in François, et al. Molecular Therapy Methods & Clinical Development (2018) Vol. 10, pp. 223-236 (incorporated by reference herein in its entirety) can be used. Relative infectivity assay as described in Provisional Application 62/745,859 filed Oct. 15, 2018) can be used.

### 4.6.19 Differential Scanning Fluorimetry

The thermal stability of proteins and virus capsids made up of proteins can be determined by differential scanning fluorimetry (DSF). DSF measures the intrinsic tryptophan and tyrosine emission of proteins as a function of temperature. The local environment of Trp and Tyr residues changes as the protein unfolds resulting in a large increase in fluorescence. The temperature where 50% of proteins are unfolded is defined as the ‘melting’ temperature (Tₘ). Fluorescence spectroscopy is described in the USP <853> and USP <1853>.

DSF data can be collected using a Promethius NTPlex Nano DSF Instrument (NanoTemper technologies, Munich, Germany). Samples can be loaded into the capillary cell at 20° C. and the temperature ramped at a rate of 1° C./min to 95° C. The signal output ratio of emission at 350 nm (unfolded) and 330 nm (unfolded) can be used to determine the Tₘ.

### 4.6.20 Injection Pressure Measurements

Injection pressures were measured using either a Flow Screen and Fluid Sensor (Viscotec America, Kennesaw, GA) or a PressureMAT-DPG with single use pressure sensor S-N-000 (PendoTECH, Princeton, NJ).

Injections into air were either performed manually or using a Legato-100 syringe pump (Kd Scientific, Holliston, MA) to apply a consistent flow rate. For injections into enucleated porcine eyes, the eyes were mounted on a Mandell eye mount (Mastel) with applied suction to adjust the introcular pressure of the eye.

### 4.6.21 Reference Compositions

The AAV aggregation level of a composition provided herein may be evaluated by comparing the composition to a reference pharmaceutical composition. In some embodiments, the reference pharmaceutical composition is a pharmaceutical composition comprising the same recombinant AAV in the same concentration as the composition being evaluated in phosphate-buffered saline. In some embodiments, the reference pharmaceutical composition is a pharmaceutical composition comprising the same recombinant AAV in the same concentration as the composition being evaluated in Dulbecco's phosphate buffered saline with 0.001% poloxamer 188, pH 7.4. In some embodiments, the reference pharmaceutical composition is a pharmaceutical composition comprising the same recombinant AAV in the same concentration as the composition being evaluated in Dulbecco's phosphate buffered saline with 4% sucrose and 0.001% poloxamer 188, pH 7.4. In some embodiments, the reference pharmaceutical composition is a pharmaceutical composition comprising the same recombinant AAV in the same concentration as the composition being evaluated in phosphate-buffered 10% sucrose diluent. In some embodiments, the reference pharmaceutical composition is a pharmaceutical composition comprising the same recombinant AAV in the same concentration as the composition being evaluated in modified DPBS with 4% sucrose formulation. In some embodiments, the reference pharmaceutical composition is a pharmaceutical composition comprising the same recombinant AAV in the same concentration as the composition being evaluated in DPBS with 0.001% P188 saline solution.

### 5. EXAMPLES

The examples in this section (i.e., section 5) are offered by way of illustration, and not by way of limitation.

### 5.1 Example 1: Preparation of Diluents Suitable to Induce Clustering of AAV

Solutions of modified DPBS with sucrose containing a recombinant adeno-associated virus (AAV) vector comprising an expression cassette encoding a transgene (Table 2) were diluted with phosphate-buffered 10% sucrose diluents (Table 3) to obtain lower ionic strength solutions. The phosphate-buffered 10% sucrose diluents have the same excipient and buffering capacity as the modified DPBS but with reduced ionic excipient sodium chloride and increased non-ionic excipient sucrose in order to reduce the ionic strength while maintaining the tonicity/osmolality in a desired range (equal to or greater than 240 mOsm/kg). A summary of the properties of the modified DPBS with sucrose and of the phosphate-buffered 10% sucrose diluent are shown in Table 4.

TABLE 2
Modified DPBS with Sucrose Formulation

| | | | | | Mass | | | Molecular |
|---|---|---|---|---|---|---|---|---|
| | | Quality | Concentration | Concentration | Fraction | Vendor and | Chemical | Weight |
| Ingredient | Function | Standard | (mg/mL) | (mM or %) | (g/kg)^{b} | Part Number | Formula | (g/mol) |
| Construct | API | Internal | Varies based | — | — | — | — | — |
| | | | on dose level | | | | | |
| Sodium | Buffering | USP, | 5.84 | 100 mM | 5.736 | Avantor, | NaCl | 58.440 |
| Chloride | Agent | Ph. Eur, | | | | 3627 | | |
| | | BP, JPE | | | | | | |
| Potassium | | USP, | 0.201 | 2.70 mM | 0.198 | Avantor, | KCl | 74.5513 |
| Chloride | | BP, | | | | 3045 | | |
| | | Ph. Eur, | | | | | | |
| | | JPE | | | | | | |
| Sodium | | USP, | 1.15 | 8.10 mM | 1.129 | Avantor, | Na₂HPO₄ | 141.960 |
| Phosphate | | Ph. Eur, | | | | 3804 | | |
| Dibasic | | JPE | | | | | | |
| Anhydrous | | | | | | | | |
| Potassium | | NF, BP, | 0.200 | 1.47 mM | 0.196 | Avantor, | KH₂PO₄ | 136.086 |
| Phosphate | | Ph. Eur | | | | 3248 | | |
| Monobasic | | | | | | | | |
| Sucrose | Cryoprotectant | USP, | 40.0 | 117 mM | 39.26 | Pfanstiehl, | C₁₂H₂₂O₁₁ | 342.3 |
| | | NF | | | | S-124-2-MC | | |
| | | Ph. Eur, | | | | | | |
| | | BP, JPE | | | | | | |
| Poloxamer | Surfactant^{a} | NF | 0.010 | 0.001% | 0.1 mL/kg | BASF, | HO(C₃H₆O)ₐ | 7680 to 9510 |
| 188 | | Ph. Eur, | | | of 10% | 50424596 | (C₂H₄O)_{b}(C₃H₆O)ₐH | |
| | | JPE | | | stock | | | |
| Water | Aqueous | WFI | Approximately | Approximately | QS to | Varies | H₂O | 18.0153 |
| | Vehicle | | 971 mg/mL | 54M | 1 kg | | | |
| | | | | | (need | | | |
| | | | | | approx. | | | |
| | | | | | 953 g/kg) | | | |

^{a}Spike 0.1 mL/L = 0.1 mL/kg of 10% stock P188. NF grade Pluronic ® F-68 (poloxamer 188) from Spectrum and Kolliphor ® P188 BIO from BASF may be used.
^{b}Volume of 1 kg of solution is approximately 982 mL (1 kg/1.0188 kg/L = 982 mL)

TABLE 3
Phosphate-buffered 10% sucrose diluent

| | | | | | | | Molecular |
|---|---|---|---|---|---|---|---|
| | | Quality | Concentration | Concentration | Vendor and | Chemical | Weight |
| Ingredient | Function | Standard | (g/L) | (mM or %) | Part Number | Formula | (g/mol) |
| Potassium | Buffering Agent | USP, BP, | 0.201 | 2.70 mM | Avantor, | KCl | 74.5513 |
| Chloride | | Ph. Eur, | | | 3045 | | |
| | | JPE | | | | | |
| Sodium | | USP, | 1.15 | 8.10 mM | Avantor, | Na2HPO4 | 141.960 |
| Phosphate | | Ph. Eur, | | | 3804 | | |
| Dibasic | | JPE | | | | | |
| Anhydrous | | | | | | | |
| Potassium | | NF, BP, | 0.200 | 1.47 mM | Avantor, | KH2PO4 | 136.086 |
| Phosphate | | Ph. Eur | | | 3248 | | |
| Monobasic | | | | | | | |
| Sucrose | Tonicity Agent | USP, NF, | 100.0 | 292 mM | Pfanstiehl, | C12H22O11 | 342.3 |
| | | Ph. Eur, | | | S-124-2-MC | | |
| | | BP, JPE | | | | | |
| Poloxamer | Surfactant^{a} | NF, Ph. Eur, | 0.010 | 0.001% | BASF, | HO(C3H6O)a | 7680 to 9510 |
| 188 | | Eur, JPE | | | 50424596 | (C2H4O)b(C3H6O)aH | |
| Water | Aqueous | WFI | QS to 1 L | | Varies | H2O | 18.0153 |
| | Vehicle | | | | | | |

^{a}Spike 0.1 mL/L = 0.1 mL/kg of 10% stock P188. NF grade Pluronic ® F-68 (poloxamer 188) from Spectrum and Kolliphor ® P188 BIO from BASF may be used.

TABLE 4
Properties of modified DPBS with sucrose formulation
buffer, phosphate-buffered 10% sucrose diluent,
and DPBS with 0.001% poloxamer 188 saline solution

| | | NaCl | Ionic | Typical |
|---|---|---|---|---|
| | Sucrose | Level | Strength | Osmolality |
| Buffer | (%) | (mM) | (mM) | (mOsm/kg) |
| modified DPBS with | 4 | 100 | 125.5 | 345 |
| sucrose formulation | | | | |
| phosphate-buffered | 10 | 0 | 25.5 | 354 |
| 10% sucrose diluent | | | | |
| DPBS with 0.001% | 0 | 137 | 162.5 | 290 |
| P188 saline solution | | | | |

### 5.2 Example 2: AAV Clustering

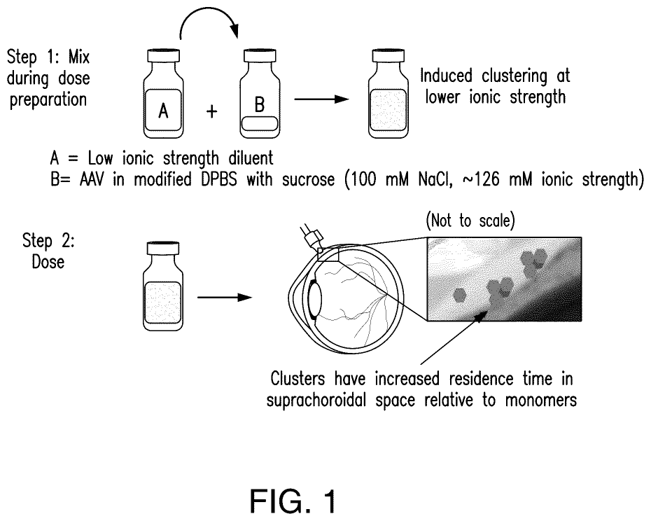

In this experiment, control solutions containing a recombinant adeno-associated virus (AAV) vector comprising an expression cassette encoding a transgene were used. In brief, solutions of modified DPBS with sucrose containing a recombinant adeno-associated virus (AAV) vector comprising an expression cassette encoding a transgene (Table 2) were diluted with phosphate-buffered 10% sucrose diluent (Table 3) to obtain AAV solutions containing lower ionic strength and salt content (FIG. 1). The dilutions resulted in AAV solutions that were diluted two-times, four-times, or eight-times.

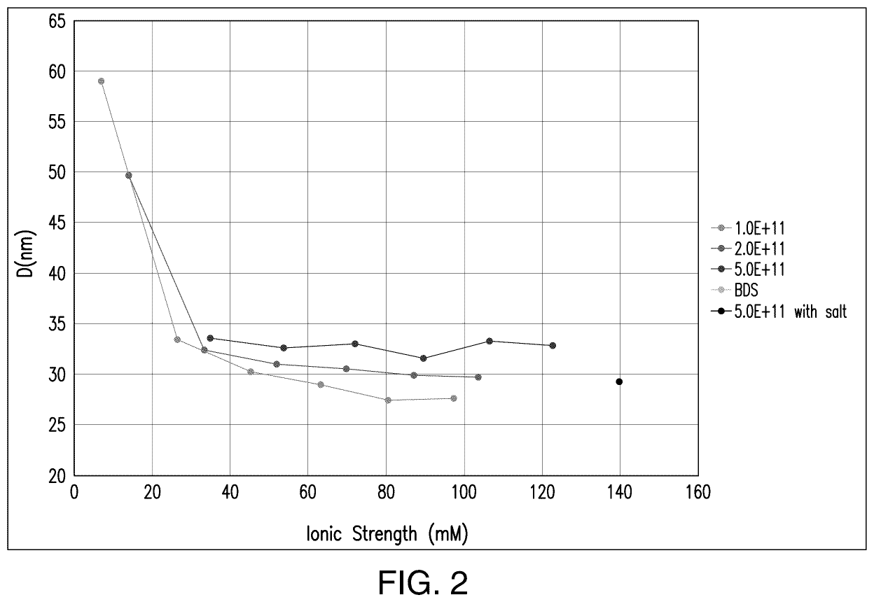

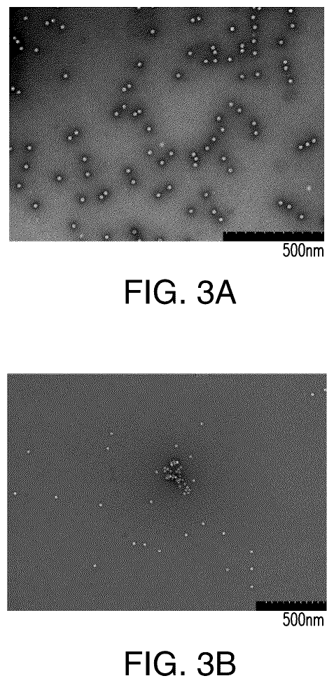

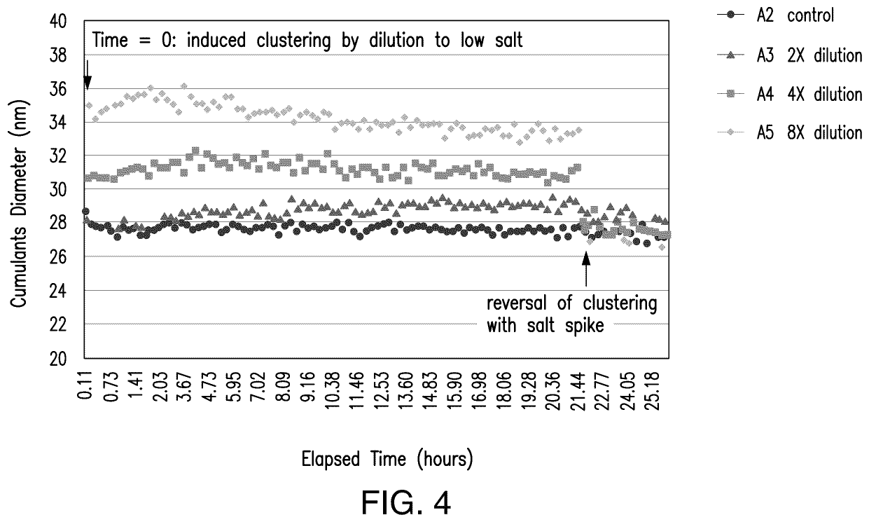

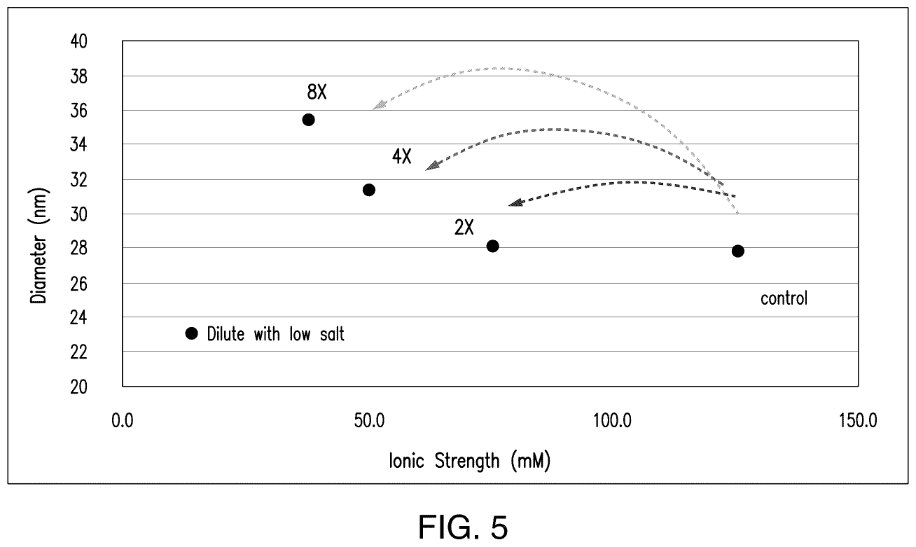

The molecular diameter (nm) of AAV was then measured in the control solutions and in the two-times, four-times, or eight-times diluted solutions. This experiment showed that as the ionic strength decreased, the molecular diameter (nm) of the AAV increased (FIG. 4 and FIG. 5). This result correlated with the fact that AAV aggregated as the ionic strength of the AAV solutions decreased (FIGS. 3A-3B). This experiment showed that liquid pharmaceutical compositions containing a recombinant adeno-associated virus (AAV) vector comprising an expression cassette encoding a transgene can be diluted in the presence of reduced ionic excipient sodium chloride solutions to induce AAV clustering (FIG. 2 and FIGS. 3A-3B). The clusters were stable at 25° C. for at least 21 hours. Table 5 shows that dilution to <25 mM sodium chloride (equivalent to <51 mM ionic strength) induced AAV clustering with an average (diameter) size increase of 2.6 nm and that dilution to 12.5 mM sodium chloride (equivalent to <38 mM ionic strength) resulted in even greater AAV clustering with and average size increase of 6.5 nm (see samples A4 and A5 in Table 5).

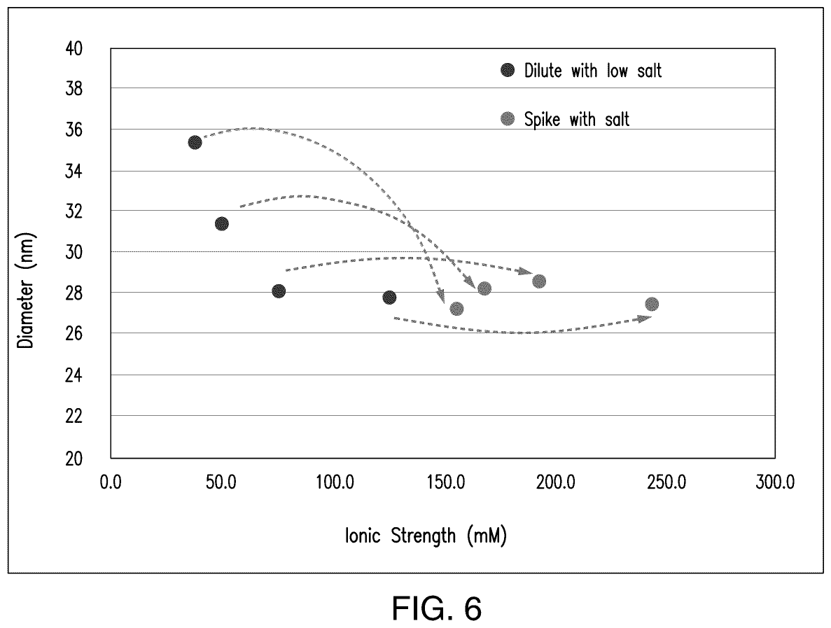

Further, this experiment showed that the molecular diameter and aggregation of AAV was reversible. After about 21 hours, solutions containing NaCl (e.g., 1 molar saline reversal solution, FIG. 4.) were added to the AAV control solutions and to the two-times, four-times, or eight-times diluted solutions to obtain ionic strengths of equal to or greater than 150 mM (FIG. 6). The reversal of AAV clusters were monitored for about 5 hours after NaCl was added. Data showed that the ionic strength increased immediately (less than 5 minutes) after NaCl solutions were added to the AAV control solutions and to the two-times, four-times, and eight-times diluted solutions (FIG. 4). AAV clusters were shown to be reversible as a result of increased ionic strength (FIG. 4 and FIG. 6). Thus, the structure and function of the AAV capsids are not irreversibly altered by the induced clustering. Data showed that AAV aggregation is reversible based on ionic strength and that suprachoroidal administration of a solution containing aggregated AAV can result in AAV becoming unaggregated or less aggregated once in contact with bodily fluids (e.g., ocular fluids or SCS fluids).

Solutions containing clustered AAV can be administered to a suprachoroidal space in an eye of a subject resulting in increased localization time at a site of injection (FIG. 1), thereby slowing clearance rates and overall clearance time. The actual rate of bolus/bleb solution composition exchange in vivo in the suprachoroidal space can be delayed so that the clusters have an enhanced retention time at site of injection, and therefore increased efficacy. Administration of a solution containing clustered AAV can slow the clearance time of the AAV from the SCS and increase the duration of time that the AAV remains at the site of injection. Aggregates of AAV allow for sustained release of the AAV particles in the SCS over a period of time.

TABLE 5
Impact of induced-clustering by dilution (2X to 8X) to lower ionic
strength and salt with phosphate-buffered 10% sucrose diluent

| | | NaCl | Ionic | Average Cumulants |
|---|---|---|---|---|
| | Dilution | Level | Strength | Diameter |
| Sample | Factor | (mM) | (mM) | (nm)^{a} |
| A2 (control) | 1 | 100 | 125.5 | 28.0 |
| A3 | 2 | 50 | 75.5 | 27.9 |
| A4 | 4 | 25 | 50.5 | 30.6 |
| A5 | 8 | 12.5 | 38.0 | 34.5 |

^{a}average diameter at 2.1 hours after induced clustering reported

### 5.3 Example 3: Optimized In Vitro Study

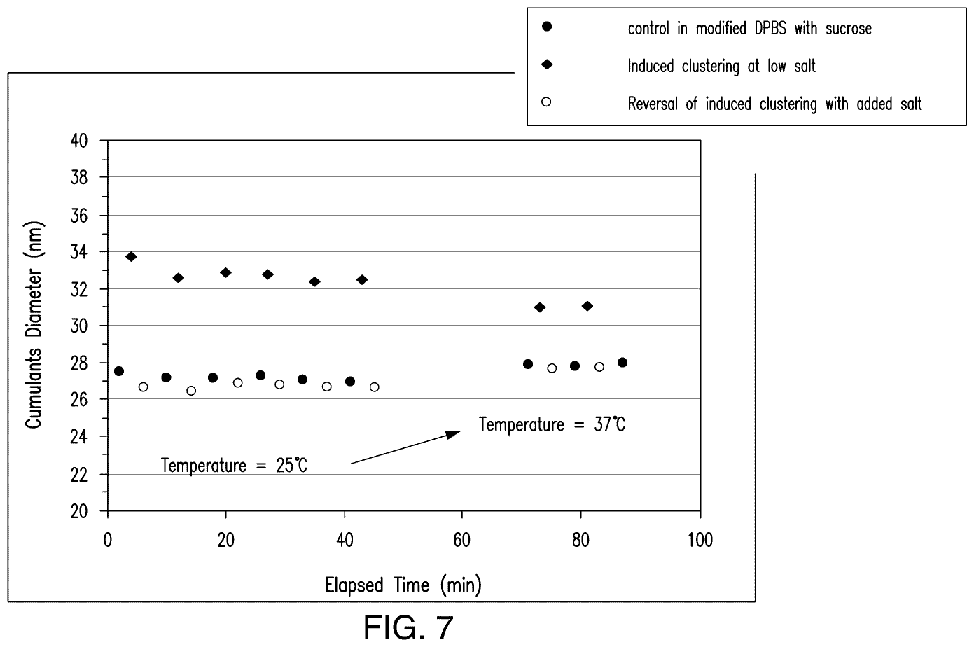

The weighted average apparent diameter of recombinant adeno-associated virus (AAV) vector comprising an expression cassette encoding a transgene in modified DPBS with sucrose and of a ten-times diluted solution of the AAV were determined by cumulants dynamic light scattering (DLS) (FIG. 7). The AAV in modified DPBS with sucrose solutions were diluted ten-times by adding phosphate-buffered 10% sucrose diluent (Table 4). This experiment also tested the weighted average apparent diameter after the ten-times diluted solutions were spiked with NaCl (e.g., by adding DPBS with 0.001% P188 saline reversal solution to the ten-times diluted solution) (Table 4 and FIG. 7).

The size of clusters were then evaluated at 25° C. for 45 min to simulate a dose preparation procedure (e.g., suprachoroidal administration). The temperature was increased to 37° C. to simulate the increase in temperature after dosing (e.g., to correlate with body temperature) and the size of the clusters were monitored for a total time of 87 min. The impact of salt (sodium chloride) levels, ionic strength, osmolality, average cumulants diameter, vector genome concentration by ddPCR and in vitro potency of the samples are shown in Table 6. This experiment showed that the diameter increased by about 5 nm for the AAV in the ten-times diluted solutions (diluted with phosphate-buffered 10% sucrose diluent) as compared to the AAV in the control modified DPBS with sucrose (compare the average cumulants diameter of sample 1 and sample 2 in Table 6). This diameter increase of AAV can affect the retention time of AAV at the site of injection after suprachoroidal administration, thus enhancing efficacy of treatment. The tonicity of the ten-times diluted solution as measured by osmolality was 357 mOsm/kg and within acceptable ranges (240<osmolality<600 mOsm/kg) for dosing into the suprachoroidal space (refer to sample 2 in Table 6). This experiment showed that no significant loss of vector genome concentration was observed between the ten-times diluted solutions (diluted with phosphate-buffered 10% sucrose diluent) and the control modified DPBS with sucrose (compare the vector genome concentration of sample 1 and sample 2 in Table 6). In addition, no significant loss of potency is expected between the samples (data not shown). This data contradicts with prior published literature (Wright et. al., 2005), which predicted that aggregation or clustering of AAV reduces AAV potency.

The fact that this experiment showed that AAV clustering did not impact potency was unexpected. This experiment showed that induced AAV clustering over a short time is reversible and therefore does not result in irreversible loss in potency. Thus, an AAV solution can be diluted to induce clustering shortly before suprachoroidal administration (FIG. 1). For example, an AAV solution can be diluted to induce AAV clustering on the same day as the suprachoroidal administration (or about 21 hours prior to suprachoroidal administration). Alternatively, diluted solutions containing clustered AAV can be stored (e.g., flash frozen, or at room temperature, or at 20° C., or at 4° C., or at −80° C.) for future use.

In some cases, a practitioner is provided with an AAV solution in one vial and a diluent solution in another vial. The practitioner can then prepare a dose by adding a specified volume of the diluent to the AAV solution (e.g., to obtain a ten-times dilution). For example, 50 μL of AAV solution at 3×10¹³ GC/mL can be diluted ten-times to 3×10¹² GC/mL with 450 μL of phosphate-buffered 10% sucrose diluent to induce clustering and then 100 μL of the ten-times diluted AAV solution can be administered into the suprachoroidal space for a total dose of 3×10¹¹ GC per eye. The final dose preparation volumes and dose can vary based on pre-clinical and clinical studies. Alternatively, a diluted AAV solution (e.g., a ten-times dilution) containing aggregated AAV can be provided in one vial to a practitioner for direct use. This way, the practitioner does not have to mix the AAV solution with a diluent prior to use.

TABLE 6
Impact of optimized induced-clustering by ten-times dilution to lower
ionic strength and salt with phosphate-buffered 10% sucrose diluent

| | | NaCl | Ionic | | Average | Vector Genome | In Vitro |
|---|---|---|---|---|---|---|---|
| Sample | | Level | Strength | Osmolality | Cumulants | Concentration by | Potency |
| # | Description | (mM) | (mM) | (mOsm/kg) | Diameter (nm)^{a} | ddPCR (GC/mL) | (%) |
| 1 | control in modified | 100 | 125.5 | 339 | 27.4 | 2.95 × 10¹³ | data not |
| | DPBS with sucrose | | | | | | shown |
| 2 | 1/10 dilution of | 10 | 35.5 | 357 | 32.4 | 2.99 × 10¹² | data not |
| | control sample 1 with | | | | | | shown |
| | phosphate-buffered | | | | | | |
| | 10% sucrose | | | | | | |
| 3 | 1/2 dilution of | 73.5 | 99 | 317 | 27.0 | 1.52 × 10¹² | data not |
| | induced clustering | | | | | | shown |
| | sample 2 into saline | | | | | | |
| | (DPBS with | | | | | | |
| | 0.001% P188) | | | | | | |

^{a}average diameter for up to 87 minutes after induced clustering, including 45 min at 25° C. followed by an increase in temperature to 37° C. for the remaining 42 min.

The threshold for AAV clustering, the diluent ionic strength, and the dilution ratio can be optimized in an analogous way for other AAV (e.g., for AAV2 and AAV9). For example, AAV8 threshold for preventing clustering was about 60 mM ionic strength. This experiment showed that a suitable induced-clustering dose preparation was achieved by diluting (10×) the AAV in modified DPBS with sucrose (ionic strength=126 mM) with phosphate-buffered sucrose (ionic-strength=26 mM) to achieve an ionic strength of about 36 mM (about half to two-thirds of the threshold ionic strength needed for preventing AAV clustering (i.e., 60 mM)). For AAV9, a robust target for induced clustering can be half to two-thirds of the AAV9 30 mM threshold for preventing clustering, which is about 15 mM to 20 mM ionic strength. Thus, for AAV9, a lower ionic strength than that of AAV8 can be desired. One way to achieve this is to reduce the buffer content of the phosphate buffered 10% sucrose diluent by a factor of five to reduce the ionic strength from 26 mM to 5.2 mM. Thus, a ten-times dilution of the AAV solution (ionic strength=126 mM) with a 5.2 mM ionic strength diluent results in a solution with a total ionic strength of about 17 mM, which is below the clustering threshold needed. Similarly, clustering of AAV2 can be achieved by reducing its ionic strength. Assuming an AAV2 is formulated with an ionic strength of between 200 mM to 600 mM, a ten-times dilution with the phosphate-buffered 10% sucrose results in an ionic strength of between 43 mM and 83 mM, which is significantly below the threshold for clustering.

Different dilution ratios and/or different diluents can be used to achieve a desired clinical clustering dose preparation (e.g., an ionic strength of about half to two-thirds of a specific clustering threshold). For AAV8 the clustering threshold is about 60 mM, so a solution target of <40 mM can be used for induced clustering. For AAV9 the threshold is about 30 mM, so a solution target of <20 mM ionic strength can be used for induced clustering. For AAV2, the threshold for clustering is 200 mM, so a solution target of <133 mM can be used for induced clustering.

### 5.4 Example 4: Effect of Liquid Formulation on Suprachoroidal Space (SCS) Thickness

The effect of liquid formulation on SCS thickness and the SCS collapse rate over time is measure in living animals (e.g., rabbit, mouse, or monkey). Different solutions having different AAV aggregation levels, or ionic strengths, or salt concentrations are used. Examples of solutions that can be used in this experiment are disclosed in the present disclosure. The initial SCS thickness at the injection site is calculated for the various pharmaceutical compositions (e.g., diluted formulation or lower ionic strength formulation), by for example, using an ultrasound imaging (see Section 4.6). The SCS thickness (e.g., SCS thickness measured before injection and after injection) depends on the level of AAV aggregation of the solutions. The SCS thickness can be measured at different time points, such as, at different time points before injection and after injection. For example, a solution comprising AAV aggregation shows a higher SCS thickness as compared to a reference solution or a solution comprising less amount of AAV aggregation. The SCS thickness is also measured over time at different positions in the eye. The level of AAV aggregation of the solutions impact the thickness of the SCS over time. For example, a solution comprising aggregated AAV increases the SCS thickness near the site of injection even when measured over time, while the SCS thickness at the injection site decreases over time when a reference solution is used. The decrease in SCS thickness at the injection site over time when using PBS or a reference solution, is accompanied by a concomitant increase in SCS thickness at adjacent sites in the SCS. The level of AAV aggregation or the ionic strength or the salt concentration of the solutions impact the duration of the SCS thickness and the localization of the SCS thickness. The AAV aggregation or the ionic strength or the salt concentration of the solution also impacts the amount of time it takes for the solution to be cleared from the SCS. For example, solutions having AAV aggregation remain in the SCS (or in the eye) for a longer period of time as compared to a reference solution.

### 5.5 Example 5: Ultrasound Imaging to Determine Suprachoroidal Space (SCS) Thickness

A high-frequency ultrasound (U/S) probe (e.g., UBM Plus, Accutome, Malvern, PA) is used to generate 2D cross-sectional images of the SCS in eyes (e.g., animal eyes) ex vivo (see Section 4.6). The cross-sectional images are generated after the eyes are injected with a solution. The solution can range in AAV aggregation, ionic strength, salt concentration, and volume. For example the volume can range from 1 μL to 500 μL. In some cases, the volume can be less than 1 μL or more than 500 μL. The solution can be an aqueous solution (e.g., water), PBS, Hank's Balanced Salt Solution (HBSS), DPBS, or any other solution of the present disclosure. The solution can further include a dye (e.g., a fluorescent dye, red-fluorescent, blue-fluorescent, blue dye, or any other dye). The solution can also include any composition, drug, agent, or virus (e.g., AAV), that can be used with the present disclosure. An U/S probe cover (e.g., Clearscan, Eye-Surgical-Instruments, Plymouth, MN) is attached to the UBM Plus to facilitate U/S image acquisition. A few minutes after injection, the U/S probe is used to acquire sagittal views around the eye (e.g., at positions 12, 1.5, 3, 4.5, 6, 7.5, 9, and 10.5 o'clock). Post-processing of the U/S B scans is performed to find the thickness from the outer sclera to the inner retina (e.g., at 1, 5, and 9 mm) posterior to the scleral spur. The mean, median, and standard deviation for each eye is calculated. Calculation of SCS thickness in ultrasound B scans can be performed by, for example, finding a line segment perpendicular to the sclera and choroid, from the outer sclera to the inner retina. The conjunctiva is excluded from the measurement. The tissue thickness is found and subtracted out, resulting in the SCS thickness.

### 5.6 Example 6: Treatment of Batten-CLN1 or CLN2-Associated Vision Loss by Suprachoroidal Injection

A subject presenting with Batten-CLN1-associated vision loss is administered AAV8 or AAV9 that encodes Palmitoyl-Protein Thioesterase 1 at a dose sufficient to produce a therapeutically effective concentration of the transgene product in the eye (e.g., vitreous humor) for three months. A subject presenting with Batten-CLN2-associated vision loss is administered AAV8 or AAV9 that encodes Tripeptidyl-Peptidase 1 at a dose sufficient to produce a therapeutically effective concentration of the transgene product in the eye (e.g., vitreous humor) for three months. The administration is done by administration to the suprachoroidal space. Several pharmaceutical compositions (e.g., diluted formulation or lower ionic strength formulation) having different AAV aggregation levels are used. The level of AAV aggregation, ionic strength, or salt concentrations of the pharmaceutical compositions (e.g., diluted formulation or lower ionic strength formulation) impact Batten-CLN2 or CLN1-associated vision loss and efficacy of treatment. Following treatment, the subject is evaluated for improvement in Batten-CLN2-associated vision loss. Following treatment, the subject is evaluated for improvement in Batten-CLN1-associated vision loss. Subjects that have the AAV administered in the SCS when a pharmaceutical composition comprising aggregated AAV is used show better improvement in Batten-CLN1 or CLN2-associated vision loss as compared to subjects that have the same pharmaceutical composition administered by subretinal injection. Subjects that have the AAV administered in the SCS when a pharmaceutical composition comprising aggregated AAV is used show better improvement in Batten-CLN1 or CLN2-associated vision loss as compared to subjects that have a reference pharmaceutical composition administered by subretinal injection, by intravitreous administration, or to the SCS.

Effects of the methods provided herein on visual deficits are measured by one or more visual acuity screenings, including OptoKinetic Nystagmus (OKN). OKN visual acuity screening uses the principles of the OKN involuntary reflex to objectively assess whether a patient's eyes can follow a moving target. The percentage change in OKN screening results before and after the said treatment is calculated.

### 5.7 Example 7: Use of an Infrared Thermal Camera to Monitor Injection in Human Patients

A subject presenting with wet AMD is administered AAV8 that encodes a transgene (e.g., by subretinal administration, suprachoroidal administration, or intravitreal administration) at a dose sufficient to produce a concentration of the transgene product at a Cmin of at least 0.330 μg/mL in the eye (e.g., vitreous humor) for three months. The AAV8 encoding the transgene can be administered using several pharmaceutical compositions (e.g., diluted formulation or lower ionic strength formulation) that have different AAV aggregation levels, by suprachoroidal administration. Subjects that have the AAV8 encoding the transgene administered in a solution comprising aggregated AAV (compared to a reference solution or compared to a solution commonly used for AAV subretinal injections) show a higher concentration of the transgene (e.g., as measured at 1 week, 2 weeks, 3 weeks, 4 weeks, 8 weeks, or 12 weeks after administration) as compared to the concentration of the transgene in subjects that have the AAV8 encoding the transgene administered in a reference solution by suprachoroidal administration, subretinal administration, or intravitreous administration. The concentration of the transgene can be measured at any time after administration of AAV8 encoding the transgene. For example, subjects that have the AAV8 administered in the SCS using a solution comprising aggregated AAV show a higher concentration of the transgene in the eye as compared to subjects that have the AAV8 administered in the SCS, or via subretinal, or via intravitreous administrations using a reference solution as measured at 1 week, 4 weeks, 2 months, or 3 months after administration of the AAV. Similarly, subjects that have the AAV8 administered in the SCS using a solution comprising aggregated AAV show a higher concentration of the transgene as compared to subjects that have the same pharmaceutical composition administered via subretinal administration. All solutions that are used in this experiment have the same amount of genome copies.

An FLIR T530 infrared thermal camera is used to evaluate the injection during the procedure and is available to evaluate after the injection to confirm either that the administration is successfully completed or misdose of the administration. Alternatively, an FLIR T420, FLIR T440, Fluke Ti400, or FLIRE60 infrared thermal camera is used. Following treatment, the subject is evaluated clinically for signs of clinical effect and improvement in signs and symptoms of wet AMD.

### 5.8 Example 8: Components in Formulation A and Formulation B

This example shows the components in Formulation A (Dulbecco's phosphate buffered saline with 0.001% poloxamer 188, pH 7.4), stored at ≤−60° C., and Formulation B (‘modified Dulbecco's phosphate buffered saline with 4% sucrose and 0.001% poloxamer 188, pH 7.4’), stored at −20° C. The comparison and impact analysis for the two Formulations is provided in Table 7. Formulation B has improved storage feasibility, without impact on the AAV product observed to date after 2 years of storage. Other pharmaceutical compositions (e.g., diluted formulation or lower ionic strength formulation) having different levels of AAV aggregation, or ionic strength, or salt concentrations are tested. Pharmaceutical compositions of the present disclosure can include, for example, one or more components from Formulation B. Pharmaceutical compositions of the present disclosure (e.g., with AAV aggregation) have improved storage feasibility, without impact on the AAV product (e.g., after 2 years of storage).

TABLE 7
Formulations A and B.

| Process Site/Stage | Formulation A | Formulation B |
|---|---|---|
| Formulation Buffer | DPBS with 0.001% Poloxamer | ‘modified DPBS with 4% Sucrose and 0.001% |
| | 188, pH 7.4. | Poloxamer 188, pH 7.4.’ |
| | Composition: | The ‘modified DPBS with 4% Sucrose and |
| | 0.2 mg/mL potassium chloride, | 0.001% Poloxamer 188, pH 7.4.’ formulation |
| | 0.2 mg/mL potassium phosphate | has 4% w/v of sucrose and a lower sodium |
| | monobasic, 8.1 mg/mL sodium | chloride level (reduced from 137 mM to 100 |
| | chloride, 1.15 mg/mL sodium | mM) to compensate tonicity. The other |
| | phosphate dibasic anhydrous, | formulation excipients and levels are identical. |
| | 0.001% (0.01 mg/mL) poloxamer | Composition: 0.2 mg/mL potassium chloride, |
| | 188, pH 7.4 | 0.2 mg/mL potassium phosphate monobasic, |
| | | 5.84 mg/mL sodium chloride, 1.15 mg/mL |
| | | sodium phosphate dibasic anhydrous, 40.0 |
| | | mg/mL (4% w/v) sucrose, 0.001% (0.01 |
| | | mg/mL) poloxamer 188, pH 7.4 |
| FDP Long-term | ≤−60° C. | −20° C. |
| Frozen Storage | | |
| Temperature | | |

Formulation B (Modified DPBS with Sucrose) includes 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4 (Table 8). In molar units, Formulation B includes 2.70 mM potassium chloride, 1.47 mM potassium phosphate monobasic, 100 mM sodium chloride, 8.1 mM sodium phosphate dibasic anhydrous, 117 mM sucrose, 0.001% (0.01 mg/mL) poloxamer 188, pH 7.4. The density of Formulation B may be 1.0188 g/mL; The osmolality of Formulation B may be approximately 345 (331-354).

TABLE 8
Formulation B with a Construct as Active Pharmaceutical Ingredient (API).

| | | | | | Mass | Vendor | | Molecular |
|---|---|---|---|---|---|---|---|---|
| | | Quality | Concentration | Concentration | Fraction | and Part | Chemical | Weight |
| Ingredient | Function | Standard | (mg/mL) | (mM or %) | (g/kg)^{b} | Number | Formula | (g/mol) |
| Construct | API | Internal | Varies based on | — | — | — | — | — |
| | | | dose level | | | | | |
| Sodium | Buffering | USP, | 5.84 | 100 mM | 5.736 | Avantor, | NaCl | 58.440 |
| Chloride | Agent | Ph. Eur, | | | | 3627 | | |
| | | BP, JPE | | | | | | |
| Potassium | | USP, BP, | 0.201 | 2.70 mM | 0.198 | Avantor, | KCl | 74.5513 |
| Chloride | | Ph. Eur, | | | | 3045 | | |
| | | JPE | | | | | | |
| Sodium | | USP, | 1.15 | 8.10 mM | 1.129 | Avantor, | Na₂HPO₄ | 141.960 |
| Phosphate | | Ph. Eur, | | | | 3804 | | |
| Dibasic | | JPE | | | | | | |
| Anhydrous | | | | | | | | |
| Potassium | | NF, BP, | 0.200 | 1.47 mM | 0.196 | Avantor, | KH₂PO₄ | 136.086 |
| Phosphate | | Ph. Eur | | | | 3248 | | |
| Monobasic | | | | | | | | |
| Sucrose | Cryoprotectant | USP, NF, | 40.0 | 117 mM | 39.26 | Pfanstiehl, | C12H22O11 | 342.3 |
| | | Ph. Eur, | | | | S-124-2-MC | | |
| | | BP, JPE | | | | | | |
| Poloxamer | Surfactant^{a} | NF, | 0.010 | 0.001% | 0.1 mL/kg | BASF, | HO(C₃H₆O)ₐ | 7680 to 9510 |
| 188 | | Ph. Eur, | | | of | 50424596 | (C₂H₄O)_{b}(C₃H₆O)ₐH | |
| | | JPE | | | 10% | | | |
| | | | | | stock | | | |
| Water | Aqueous | WFI | Approximately | Approximately | QS | Varies | H₂O | 18.0153 |
| | Vehicle | | 971 mg/mL | 54M | to 1 kg | | | |
| | | | | | (need | | | |
| | | | | | approx. | | | |
| | | | | | 953 g/kg) | | | |

^{a}Spike 0.1 mL/L = 0.1 mL/kg of 10% stock P188. NF grade Pluronic ® F-68 (poloxamer 188) from Spectrum and Kolliphor ® P188 BIO from BASF may be used.
^{b}Volume of 1 kg of solution is approximately 982 mL (1 kg/1.0188 kg/L = 982 mL)

### 5.9 Example 9: Comparison of Formulation A and Formulation B in Long Term Stability

This example shows the comparison of Formulation A and Formulation B in long term stability. Formulation A and B had similar long-term frozen stability at −80° C., and Formulation B was also stable at −20° C. The ‘modified dPBS with 4% sucrose’ formulation B maintained potency for 12 months at −20° C. and −80° C. Other pharmaceutical compositions (e.g., diluted formulation or lower ionic strength formulation) having different levels of AAV aggregation are tested. Pharmaceutical compositions of the present disclosure (e.g., comprising aggregated AAV) are stable at −20° C. and at −80° C. Pharmaceutical compositions comprising AAV aggregation maintains potency for 12 months at −20° C. and −80° C. Pharmaceutical compositions of the present disclosure (e.g., comprising aggregated AAV) can include, for example, one or more components from Formulation B.

### 5.10 Example 10: Comparison of Formulation A and Formulation C In-Vitro Potency

This example shows the comparison of Formulation A and Formulation C in long term stability. Formulation C is a variant of the ‘modified dPBS with sucrose’ with 60 mM NaCl and 6% sucrose. Formulation C includes 0.2 mg/mL potassium chloride, 0.2 mg/mL potassium phosphate monobasic, 3.50 mg/mL sodium chloride, 1.15 mg/mL sodium phosphate dibasic anhydrous, 60.0 mg/mL (6% w/v) sucrose, 0.001% (0.01 mg/mL) poloxamer 188, pH 7.4.

Formulation C was stable for 2 years at −20° C. The reference formulation A (dPBS) was not stable at −20° C. Formulations B and C may have comparable and superior long-term stability at −20° C. Other pharmaceutical compositions (e.g., diluted formulation or lower ionic strength formulation) having different levels of AAV aggregation are tested. Pharmaceutical compositions of the present disclosure (e.g., comprising AAV aggregation) can include, for example, one or more components from Formulation B or Formulation C. Pharmaceutical compositions of the present disclosure (e.g., comprising AAV aggregation) are stable for 2 years at −20° C.

### 5.11 Example 11: Pharmacodynamic, Biodistribution, and Tolerability Study in Cynomolgus Monkeys Using Different Suprachoroidal Formulations

The objective of this study is to evaluate the biodistribution, pharmacodynamics (transgene concentration), and tolerability of different formulations comprising AAV8 when administered as a single dose via suprachoroidal injection to Cynomolgus monkeys. After dosing, animals are observed postdose for at least 4 weeks. One group is also administered a high volume of the formulations. Some of the formulations have varying AAV aggregation levels, ranging from low aggregation to high aggregation. Some of the formulations have varying ionic strength levels, ranging from low ionic strength to high ionic strength. For example, Formulation 1 has low AAV aggregation level, Formulation 2 has intermediate AAV aggregation level, and Formulation 3 has high AAV aggregation level. The group assignment and dose levels are shown in Table 9. The test article is an AAV8 construct. The control article is a placebo. The formulations and the controls can be stored in a freezer between −60° C. and −80° C. and thawed at room temperature on the day of use, or stored at room temperature if used on the day of formulation, or stored in a refrigerator between 2° C. and 8° C.

TABLE 9
Group Assignment and Dose Levels

| | | | | Dose | Number |
|---|---|---|---|---|---|
| | Dose | Dose | Dose | Concen- | of |
| | Regimen | Regimen | level^{b} | tration | Animals |
| Group | Left Eye | Right Eye | (GC/eye) | (GC/mL) | (Females) |
| Control 1^{a} | Control | Control | 0 | 0 | 41 |
| | Article 1 | Article 1 | | | |
| Control 2 | Control | Control | 0 | 0 | 1 |
| | Article 2 | Article 2 | | | |
| Control 3 | Control | Control | 0 | 0 | 1 |
| | Article 3 | Article 3 | | | |
| Formula- | Test | Test | 3 × 10¹¹ | 3 × 10¹² | 4 |
| tion 1 | Article 1 | Article 1 | | | |
| Formula- | Test | Test | 3 × 10¹¹ | 3 × 10¹² | 4 |
| tion 2 | Article 2 | Article 2 | | | |
| Formula- | Test | Test | 3 × 10¹¹ | 3 × 10¹² | 4 |
| tion 3 | Article 3 | Article 3 | | | |
| High | Test | Test | 3 × 10¹¹ | 1.5 × 10¹² | 4 |
| volume | Articles 1, | Article 1, | | | |
| formula- | 2, or 3 | 2, or 3 | | | |
| tion | | | | | |

GC = Genome copies
^{a}Group 1 is administered control article only.
^{b}Dose levels are based on a dose volume of 100 μL/eye for Formulations 1-3, and volume of 200 μL/eye for the high volume formulation group. Each eye is administered two injections.
c all animals are sacrificed on day 29 of the dosing phase.

Antibody Prescreening at Animal Supplier: blood (at least 1 mL) from about 90 female monkeys is collected from each animal via a femoral vein and placed into tubes containing no anticoagulant. Another vein may be used for collection, as needed. Animals are selected as study candidates based on the pre-screening results. Blood is allowed to clot at room temperature and centrifuged within 1 hour to obtain serum. Serum is divided into 2 aliquots and placed into cryovials and maintained on dry ice prior to storage at approximately −70° C. Samples are shipped overnight on dry ice for analysis. Samples are then analyzed for anti-AAV8 neutralizing antibodies (NAbs) by any acceptable method. Animals are selected for shipment based on anti-AAV8 Nab results.

Dose Administration: animals are fasted overnight and anesthetized with ketamine and dexmedetomidine prior to suprachoroidal injection. In brief, a single suprachoroidal injection of 100 μL (or 2 injections of 50 μL each) is administered to each eye (between 3 and 4 mm from the limbus) over 5 to 10 seconds. For the high volume formulation, 200 μL per eye is administered. The formulations are administered with Clearside SCS Microinjectors. The microneedle size can vary depending on the viscosity of the formulation. In some cases a 30-gauge microneedle is used. Injections in the right eye is administered in the superior temporal quadrant (i.e., between the 10 o'clock and 11 o'clock positions. Injections in the left eye is administered in the superior temporal quadrant (i.e., between the 1 o'clock and 2 o'clock positions). Following the injection, the needle is kept in the eye for approximately 5 seconds before being withdrawn. Upon withdrawal of the micro needle, a cotton-tipped applicator (dose wipe) is placed over the injection site for approximately 10 seconds. A topical antibiotic (e.g. Tobrex® or appropriate substitute) is instilled in each eye following dosing. Each dosing time is recorded as the time at the completion of each injection. The right eye is dosed first, followed by the left eye.

Ophthalmic Procedures: ophthalmic examinations (e.g., on days 4, 8, 15, and 29 post administration) are conducted. Animals are examined with a slit lamp biomicroscope and indirect ophthalmoscope. The adnexa and anterior portion of both eyes are examined using a slit lamp biomicroscope. The ocular fundus of both eyes are examined (where visible) using an indirect ophthalmoscope. Prior to examination with the indirect ophthalmoscope, pupils are dilated with a mydriatic agent (e.g., 1% tropicamide). Intraocular pressure is measured on the day of administration (within 10 minutes prior to dosing) and, for example, on days 4, 8, 15, and 29. Rebound tonometry (TonoVet) can be used to evaluate ocular pressure. Ocular photography is performed around week 4. Photographs are taken with a digital fundus camera. Color photographs are taken of each eye to include stereoscopic photographs of the posterior pole and nonstereoscopic photographs of two midperipheral fields (temporal and nasal). Photographs of the periphery is also performed. Further, autofluorescence imaging with indocyanine green is conducted to document spread of dose (e.g., on days one and two).

Anti-AAV8 Neutralizing Antibody Analysis: blood samples from each animal taken from a femoral vein at different time points (e.g., prior to administration, on day of administration, and on days after administration) are held at room temperature and allowed to clot for at least 30 minutes prior to centrifugation. Samples are centrifuged within 1 hour of collection, and serum is harvested. Following harvesting, samples are placed on dry ice until stored between −60° C. and −80° C. Serum analysis for AAV8 antibodies is then performed using a qualified neutralizing antibody assay.

Anti-AAV8-Transgene Product Antibody Analysis: blood samples are taken as discussed above and serum samples are analyzed for antibodies to the AAV8-transgene using any assay of the present disclosure or any acceptable assay. For AAV8-transgene analysis, blood samples are taken as described above at least two weeks prior to administration, on day 15, and on the day of animal sacrifice (Day 29). 50 μL from the anterior chamber is collected before dose administration. Samples from the aqueous humor and the vitreous humor can be collected at the terminal necropsy. Serum samples can be collected pre-dose, on Day 15, and prior to necropsy. Samples are then analyzed by any assay of the present disclosure or any applicable assay or method (e.g., for transgene concentration).

Aqueous Humor Collection: approximately 50 μL is removed from each eye at least 2 weeks prior to administration, on day 15, and on the day the animals are sacrificed. Aqueous humor samples from each eye is placed into separate tubes with Watson barcoded labels, snap frozen in liquid nitrogen, and placed on dry ice until stored between −60° C. and −80° C.

Post Aqueous Tap Medication Regimen: the objective of this treatment regimen is to provide palliative treatment related to aqueous humor collection procedures. The treatment objective following collection days is to provide appropriate palliation of adverse events (e.g., discomfort). Animals are tested for ocular pain and side effects.

TABLE 10
Medication Regimen

| Days | Drug (Dose Level) | Dose Route | Interval |
|---|---|---|---|
| Day of | Flunixin meglumine | IM | Prior to sedation |
| sampling | (2 mg/kg) | | for collection |
| Day of | Buprenorphine (0.05 | IM | Upon recovery from |
| sampling | mg/kg) | | anesthesia; 5 to 7 |
| | | | hours later, and at |
| | | | least 16 hours later |
| | | | (from the first |
| | | | injection) |
| Day of | 1% Atropine sulfate | Topical | After collection |
| sampling | solution^{a} | | procedures |
| Day of | Neo-Poly-Dex | Topical | After collection |
| sampling | ointment^{b} | | procedures |
| 1 day after | 1% Atropine sulfate | Topical | Once |
| sampling | solution^{a} | | |
| 1 day after | Neo-Poly-Dex | Topical | BID |
| sampling | ointment^{b} | | |
| 2 days after | 1% Atropine sulfate | Topical | Once |
| sampling | solution^{a} | | |
| 2 days after | Neo-Poly-Dex | Topical | BID |
| sampling | ointment^{b} | | |

BID = Twice daily (at least 6 hours apart);
IM = Intramuscular injection
^{a}Applied as 1 to 2 drops of solution to each eye from which samples were collected.
^{b}Applied as an approximate 0.25 inch strip to each eye from which samples were collected.

Termination of Study: animals are anesthetized with sodium pentobarbital and exsanguinated on Day 29.

Necropsy Collections of Aqueous Humor and Vitreous Humor: up to 50 μL per eye and up to 100 μL per eye is removed from the aqueous humor and the vitreous humor, respectively. Following exsanguination, eyes are enucleated and aqueous humor and vitreous humor samples are collected from each eye. Vitreous humor samples are divided into 2 approximately equal aliquots and aqueous humor samples are stored as one aliquot. After each collection, the right eyes of animals are injected with modified Davidson's fixative until turgid. Eyes are stored in modified Davidson's fixative for 48 to 96 hours, and then transferred to 10% neutral-buffered formalin. Samples are flash frozen and stored between −60° C. and −80° C. Aqueous and vitreous samples are analyzed for transgene concentration.

Ocular Tissue Collection for Biodistribution: following exsanguination, the left eye from all animals and right eye from two animals (depending on survival) from the various formulation groups are enucleated and tissues are collected. Tissues are collected into separate tubes with Watson barcoded labels. Collected tissue includes choroid with retinal pigmented epithelium, cornea, iris-ciliary body, optic chiasm, optic nerve, retina, sclera, and posterior eye cup. Eyes are divided into four approximately equal quadrants (superior-temporal to include the area of the dose site, superior-nasal, inferior-temporal, and inferior nasal to include the area of the dose site). From each quadrant, one sample is taken using an 8 mm biopsy punch. Samples are stored between −60° C. and −80° C. Samples are analyzed for vector DNA or RNA using a qPCR or qRT-PCR method.

Non-Ocular Tissue Collection for Biodistribution: two samples of approximately 5 mm×5 mm×5 mm is collected from the right brain hemisphere (e.g., cerebellum (lateral), cerebellum (dorsal), frontal cortex (Brodmann area 4), frontal cortex (Brodmann area 6), occipital cortex (cortical surface), occipital cortex (parenchyma)), ovary, heart, kidney, lacrimal gland (left), liver (left lateral lobe), lung (left caudal lobe), lymph node (parotid), lymph node (mandibular), pituitary gland, salivary gland (mandibular), spleen, thymus, dorsal root ganglia (cervical, left), dorsal root ganglia (lumbar, left), and dorsal root ganglia (thoracic, left). Samples are stored between −60° C. and −80° C.

Histology: right eye and right optic nerve from animals are sectioned at a nominal 5 μm and stained with hematoxylin and eosin. Eye tissues are sectioned to facilitate examination of the fovea, injection site region, macula, optic disc, and optic nerve. A single, vertical section is taken through the approximate center of the inferior calotte. This results in one slide/block/eye (three slides per eye total). Further, digital scans (virtual slides) can be prepared from selected microscopic slides.

Data Evaluation and Statistical Analysis: statistical data analyses are calculated using means and standard deviations. Means and standard deviations are calculated for absolute body weight, body weight change, and intraocular pressure measurements.

### 5.12 Example 12: Pharmacodynamic, Biodistribution, and Tolerability Study in Cynomolgus Monkeys Using Different Suprachoroidal Formulations

The objective of this study was to evaluate the biodistribution (DNA and mRNA), pharmacodynamics (transgene concentration), and tolerability of clustering formulations comprising AAV8 construct when administered as a single dose via suprachoroidal injection to Cynomolgus monkeys. After dosing, animals were observed postdose for at least 4 weeks. Each group was administered two injections to achieve the same dose volume. The group assignment and dose levels were shown in Table 11. The test article was AAV8 construct. The control article was a placebo.

TABLE 11
Group Assignment and Dose Levels

| | | | Dose | Dose | Number of | |
|---|---|---|---|---|---|---|
| | Dose Regimen | | Level^{b} | Concentration | Animals^{c} | |
| Group^{a} | Left Eye | Right Eye | (gc/eye) | (gc/mL) | Males | Females |
| 1 | Control | Control | 0 | 0 | NA | 1 |
| | Article 3 | Article 3 | | | | |
| 2 | Test | Test | 3 × 10¹¹ | 3 × 10¹² | 3 | 1 |
| | Article 3 | Article 3 | | | | |

gc = genome copies
^{a}Group 1 was administered control articles only.
^{b}Dose levels for Groups 1 and 2 were based on a dose volume of 100 μL/eye/dose administered as two 50 μL injections.
^{c}All animals were sacrificed on Day 29 of the dosing phase.

Dose Administration: the preparation of test articles and control articles are shown in Table 12. The test articles and control articles were stored in a freezer between −60° C. and −80° C. and thawed at room temperature on the day of use. The formulations were thawed at room temperature and stored at room temperature until prepared by diluting stock concertation and used for syringe filling. Animals were anesthetized with ketamine and dexmedetomidine prior to suprachoroidal injection. In administration, two suprachoroidal injections of 50 μL (Groups 1 and 2) was administered to each eye (between 3 and 4 mm from the limbus) over 10 to 15 seconds. The syringe and microneedle size are shown in Table 12. The first injection in the right eye was administered in the superior temporal quadrant (i.e., between the 10 o'clock and 11 o'clock positions), and the second injection in the right eye (as applicable) was administered in the inferior nasal quadrant (i.e., between the 4 o'clock and 5 o'clock positions). The first injection in the left eye was administered in the superior temporal quadrant (i.e., between the 1 o'clock and 2 o'clock positions), and the second injection in the left eye (as applicable) was administered in the inferior nasal quadrant (i.e., between the 7 o'clock and 8 o'clock positions). Following the injection, the needle was kept in the eye for approximately 30 seconds before being withdrawn. Upon withdrawal of the micro needle, a cotton-tipped applicator (dose wipe) was placed over the injection site for approximately 10 seconds.

TABLE 12
Preparation of Test and Vehicle Control Articles

| | Formulation | Composition | Syringe Preparation |
|---|---|---|---|
| Test | Clustering | In stock Final Drug Product | 1-mL BD syringe (309628) |
| Article | formulation | (FDP) to be mixed with Diluent^{a} | with a vial adapter (West |
| 3 | | at 100 μL:900 μL ratio on Day | 8070101) were used for |
| | | 1 of the dosing phase to achieve | preparing the formulation by |
| | | 3 × 10¹² genome copies (GC)/mL | diluting the FDP with Diluent. |
| | | | For injection the following were |
| | | | used: Clearside microinjector |
| | | | syringe (REF CLS-HN001) and |
| | | | microneedle (Clearside |
| | | | Biomedical, Inc., Part No. |
| | | | CLSD0707 CLS-A700) |
| Control | Placebo | 10% modified DPBS with | For injection the following were |
| Article | clustering | sucrose (5.84 mg/mL sodium | used: Clearside microinjector |
| 3 | formulation | chloride, 0.201 mg/mL | syringe (REF CLS-HN001) and |
| | (vehicle) | potassium chloride, 1.15 mg/mL | microneedle (Clearside |
| | | sodium phosphate dibasic | Biomedical, Inc., Part No. |
| | | anhydrous, 0.200 mg/mL | CLSD0707 CLS-A700) |
| | | potassium phosphate | |
| | | monobasic, 40.0 mg/mL (4% | |
| | | w/v) sucrose, 0.001% (0.01 | |
| | | mg/mL) poloxamer 188, pH 7.4) | |
| | | and 90% Diluent | |

^{a}Phosphate-Buffered 10% Sucrose Diluent: 2.70 mM potassium chloride, 8.10 mM sodium phosphate dibasic anhydrous, 1.47 mM potassium phosphate monobasic, 292 mM sucrose, 0.001% (0.01 mg/mL) poloxamer 188, pH 7.4

Aqueous Humor Collection: approximately 50 μL was removed from each eye at least 2 weeks prior to administration, on Day 15, and on the day the scheduled sacrificed (Day 29). Aqueous humor samples from each eye were placed into separate tubes with Watson barcoded labels, snap frozen in liquid nitrogen, and placed on dry ice until stored between −60° C. and −80° C. Samples were analyzed for transgene product concentration by a validated method.

Termination of Study: animals were anesthetized with sodium pentobarbital and exsanguinated on Day 29.

Necropsy Collections of Aqueous Humor and Vitreous Humor: Following exsanguination, eyes were enucleated and aqueous humor and vitreous humor samples were collected from both eyes. Following collection, samples were flash-frozen and stored between −60° C. and −80° C. Aqueous and vitreous samples were analyzed for transgene concentration by a validated method.

Ocular Tissue Collection for Biodistribution: following exsanguination, the right eye from each animal and the left eye from the last two animals (depending on survival) in Group 2 were enucleated and tissues were collected. Collected tissue included choroid with retinal pigmented epithelium, retina, and sclera. Tissues were collected using ultra-clean procedures as described above, and rinsed with saline and blotted dry. Samples were flash-frozen and stored between −60° C. and −80° C. Samples were analyzed for vector DNA or RNA using a qPCR or qRT-PCR method.

Comparator study: in a Cynomolgous monkey study conducted analogously to the protocols described in this Example, a control formulation (Control Article 3.5) was injected to the SCS of each eye (temporal superior and nasal inferior injection with microinjector). The control formulation does not induce AAV clustering.

TABLE 13
Preparation of Control Formulation

| | Formulation | Composition | Syringe Preparation |
|---|---|---|---|
| Control | Control SCS | Modified DPBS with sucrose (5.84 mg/mL | Clearside |
| Article | formulation | sodium chloride, 0.201 mg/mL potassium | microinjector syringe |
| 3.5 | | chloride, 1.15 mg/mL sodium phosphate dibasic | as described in Table 9 |
| | | anhydrous, 0.200 mg/mL potassium phosphate | |
| | | monobasic, 40.0 mg/mL (4% w/v) sucrose, | |
| | | 0.001% (0.01 mg/mL) poloxamer 188, pH 7.4) | |

The control formulation also contained AAV8 construct and was dosed at 3×10¹¹ gc/eye in 100 μL/eye/dose (two 50 μL injections).

Data Evaluation and Statistical Analysis: statistical data analyses were calculated using means and standard deviations. Transgene product (protein) in aqueous humor was assessed at 15 and 29 days, otherwise TP, DNA and RNA was assessed in vitreous humor at 29 days.

### Results

TABLE 14
Aqueous Humor Transgene Product (ng/mL)

| | Control Article 3- | | Test Article 3- | | Control Article 3.5- | |
|---|---|---|---|---|---|---|
| | placebo | | two injections | | control formulation | |
| | (TP ng/mL) | | (TP ng/mL) | | (TP ng/mL) | |
| | 15 days | 29 days | 15 days | 29 days | 15 days | 29 days |
| Avg. | 0^{a} | 0^{a} | 7.04 | 14.25 | 2.79 | 3.69 |
| Median | | | 6.83 | 13.5 | 3.105 | 4.16 |

^{a}when values were below limit of quantification (<0.100 ng/mL), a value of “0” was assigned for calculation of descriptive statistics.

Test article 3 (clustering formulation) injected into the SCS at the temporal superior and nasal inferior locations of the eye resulted in greater transgene product (TP) concentration in aqueous humor compared to the Control Formulation.

TABLE 15
Vitreous Humor Transgene Product (ng/mL)

| | Control Article 3- | Test Article 3- | Control Article 3.5- |
|---|---|---|---|
| | placebo | two injections | control formulation |
| | (TP ng/mL) | (TP ng/mL) | (TP ng/mL) |
| Avg. | 0^{a} | 30.778 | 17.99 |

^{a}when values were below limit of quantification (<0.100 ng/mL), a value of “0” was assigned for calculation of descriptive statistics.

Test article 3 injected into the SCS at the temporal superior and nasal inferior locations resulted in greater concentrations of transgene product in the VH compared the Control Formulation. Vitreous humor transgene product concentration was higher overall than TP found in aqueous humor 29 days following injection.

TABLE 16
Serum Transgene Product (ng/mL)

| | Control Article 3- | Test Article 3- | Control Article 3.5- |
|---|---|---|---|
| | placebo | two injections | control formulation |
| | (TP ng/mL) | (TP ng/mL) | (TP ng/mL) |
| Avg. | 0^{a} | 1.105 | 0.44 |

^{a}when values were below limit of quantification (<0.100 ng/mL), a value of “0” was assigned for calculation of descriptive statistics.

The injections of Test article 3 or Control formulation containing AAV8 construct into the SCS produced minimal titers of transgene product in the serum.

TABLE 17
DNA or RNA (copies/μg) Biodistribution in Tissues

| | Control Article 3- | | Test Article 3- | | Control Article 3.5- | |
|---|---|---|---|---|---|---|
| | placebo | | two injections | | control formulation | |
| | (copies/μg) | | (copies/μg) | | (copies/μg) | |
| | DNA | mRNA | DNA | mRNA | DNA | mRNA |
| Retina (Avg.) | Nt | nt | 2.21 × 10⁵ | 6.72 × 10⁵ | 5.61 × 10³ | nt |
| RPE/Choroid | Nt | nt | 1.43 × 10⁸ | 3.11 × 10⁵ | 3.97 × 10⁶ | nt |
| (Avg.) | | | | | | |
| Sclera (Avg.) | Nt | nt | 8.32 × 10⁷ | 5.32 × 10⁷ | 7.92 × 10⁷ | nt |

nt = not tested

Test Article 3 (Clustering formulation) had an impact on delivery to the retina and choroid, compared to the Control formulation.

### 5.13 Example 13: Suprachoroidal Formulation: Pharmacodynamic Biodistribution, and Toxicity Study in Pigs

This example relates to the evaluation of pharmacodynamic/biodistribution and toxicity of different formulations of a test article (e.g., AAV.GFP) in animals (e.g., minipigs, such as Yucatan minipig) after a single suprachoroidal injection. In brief, three (3) pigs receive each test article formulation via bilateral suprachoroidal injections into the superior temporal quadrant (performed once). The animals are analyzed at least twice daily for signs of overt discomfort such as severe blepharospasm, severe conjunctival hyperemia, epiphora, excessive rubbing at the eye, and not eating. If these conditions persist for 12 hours then the pigs are humanely euthanized.

TABLE 18
Experimental Design

| | | | Dose | | | |
|---|---|---|---|---|---|---|
| | Number of | Test Article | (GC/Eye)/ | | Endpoint | |
| Group | Animals | (OU) | Volume | Route | Parameters | Euthanasia |
| 0 | 1 | AAV.CAG.GFP | 3 × 10¹¹ | SCS | Ocular | Day 29 |
| | | Modified DPBS | in 100 μl | | Examination/ | OU: Fresh |
| | | with Sucrose | | | Tonometry: | frozen |
| | | | | | Baseline, Day 8, | collection |
| | | | | | 14, 21, 29 | |
| | | | | | EDI-Optical | |
| | | | | | Coherence | |
| | | | | | Tomography (b- | |
| | | | | | scans) & Blue | |
| | | | | | Autofluorescence | |
| | | | | | (en face): | |
| | | | | | Baseline, | |
| | | | | | Immediately Post- | |
| | | | | | Dose, Days 14, 21, | |
| | | | | | and 29 | |
| | | | | | Color & Blue | |
| | | | | | Fundus Imaging: | |
| | | | | | Baseline, Days 14, | |
| | | | | | and 29 | |
| | | | | | Serum Sampling: | |
| | | | | | Baseline and Day | |
| | | | | | 29 | |
| | | | | | Whole Blood | |
| | | | | | Sampling: | |
| | | | | | Baseline and Day | |
| | | | | | 29 | |
| 1 | 3 | AAV.CAG.GFP | 3 × 10¹¹ | SCS | Ocular | Day 29 |
| | | Modified DPBS | in 100 μl | | Examination/ | N = 5 eyes per |
| | | with Sucrose | | | Tonometry: | group: Harvest |
| | | | | | Baseline, Day 8, | cornea, iris- |
| | | | | | 14, 21, 29 | ciliary body, |
| | | | | | EDI-Optical | retina, |
| | | | | | Coherence | choroid/RPE, |
| | | | | | Tomography (b- | sclera, optic |
| | | | | | scans) & Blue | nerve, optic |
| | | | | | Autofluorescence | chiasm |
| | | | | | (en face): | N = 1 eye per |
| | | | | | Baseline, | group: Collect |
| | | | | | Immediately Post- | eye for H&E |
| | | | | | Dose, Days 14, 21, | and GFP IHC |
| | | | | | and 29 | Non-ocular |
| | | | | | Color & Blue | tissues: collect |
| | | | | | Fundus Imaging: | for |
| | | | | | Baseline, Days 14, | biodistribution |
| | | | | | and 29 | |
| | | | | | Serum Sampling: | |
| | | | | | Baseline, and Day | |
| | | | | | 29 | |
| | | | | | Whole Blood | |
| | | | | | Sampling: | |
| | | | | | Baseline and Day | |
| | | | | | 29 | |
| 2 | 3 | AAV.CAG.GFP | 3 × 10¹¹ | SCS | Ocular | Day 29 |
| | | Gel Formulation | in 100 μl | | Examination/ | N = 5 eyes per |
| | | | | | Tonometry: | group: Harvest |
| | | | | | Baseline, Day 8, | cornea, iris- |
| | | | | | 14, 21, 29 | ciliary body, |
| | | | | | EDI-Optical | retina, |
| | | | | | Coherence | choroid/RPE, |
| | | | | | Tomography (b- | sclera, optic |
| | | | | | scans) & Blue | nerve, optic |
| | | | | | Autofluorescence | chiasm |
| | | | | | (en face): | N = 1 eye per |
| | | | | | Baseline, | group: Collect |
| | | | | | Immediately Post- | eye for H&E |
| | | | | | Dose, Days 14, 21, | and GFP IHC |
| | | | | | and 29 | Non-ocular |
| | | | | | Color & Blue | tissues: collect |
| | | | | | Fundus Imaging: | for |
| | | | | | Baseline, Days 14, | biodistribution |
| | | | | | and 29 | |
| | | | | | Serum Sampling: | |
| | | | | | Baseline, and Day | |
| | | | | | 29 | |
| | | | | | Whole Blood | |
| | | | | | Sampling: | |
| | | | | | Baseline and Day | |
| | | | | | 29 | |
| 3 | 3 | AAV.CAG.GFP | 3 × 10¹¹ | SCS | Ocular | Day 29 |
| | | Clustering | in 100 μl | | Examination/ | N = 5 eyes per |
| | | Formulation | | | Tonometry: | group: Harvest |
| | | | | | Baseline, Day 8, | cornea, iris- |
| | | | | | 14, 21, 29 | ciliary body, |
| | | | | | EDI-Optical | retina, |
| | | | | | Coherence | choroid/RPE, |
| | | | | | Tomography (b- | sclera, optic |
| | | | | | scans) & Blue | nerve, optic |
| | | | | | Autofluorescence | chiasm |
| | | | | | (en face): | N = 1 eye per |
| | | | | | Baseline, | group: Collect |
| | | | | | Immediately Post- | eye for H&E |
| | | | | | Dose, Days 14, 21, | and GFP IHC |
| | | | | | and 29 | Non-ocular |
| | | | | | Color & Blue | tissues: collect |
| | | | | | Fundus Imaging: | for |
| | | | | | Baseline, Days 14, | biodistribution |
| | | | | | and 29 | |
| | | | | | Serum Sampling: | |
| | | | | | Baseline, and Day | |
| | | | | | 29 | |
| | | | | | Whole Blood | |
| | | | | | Sampling: | |
| | | | | | Baseline and Day | |
| | | | | | 29 | |

### Formulation:

### Formulation 1 (for Group 1):

Test Article: Test Article (TA) in modified DPBS with Sucrose
Label Name: AAV.CAG.GFP Control FDP
Concentration 3×10¹² GC/mL
Storage Conditions: −80° C.
Quantity: 1 vial per animal, plus 2 spares; 5 total

### Formulation 2 (for Group 2):

Test Article: TA in thermoresponsive gel formulation (“Gel Formulation”)
Label Name: AAV.CAG.GFP Gel Formulation
Concentration 3×10¹² GC/mL
Storage Conditions: −80° C.
Quantity: 1 vial per animal, plus 1 spare; 4 total

### Components for Formulation 3 (for Group 3):

A Test Article: Clustering stock
Label Name: AAV.CAG.GFP Cluster Stock
Concentration 3×10¹³ GC/mL
Storage Conditions: −80° C.
Quantity: 1 vial per animal, plus 2 spares; 5 total
Test Article: Clustering diluent
Label Name: Cluster Diluent
Storage Conditions: −80° C.
Quantity: 1 vial per animal, plus 4 spares; 7 total
Test Article: Empty vial
Label Name: EMPTY VIAL
Storage Conditions: −80° C.
Quantity: 1 vial per animal, plus 4 spares; 7 total

Instructions for Preparing Formulation 3: in addition to the 3 components listed above, 3 vial adapters, 4 BD syringes, and 2 needles are required for preparation of the test article.

### Preparation Steps are as Follows:

1. Attach vial adapter to Clustering Stock and Clustering Diluent vials
2. Withdraw active from Clustering Stock vial (˜200 μL)
3. Prime clustering stock to 100 μL
4. Add 100 μL of clustering stock to Empty Vial and remove syringe.
5. Withdraw clustering diluent from Clustering Diluent vial (˜1100 μL)
6. Prime clustering diluent to 900 μL
7. Add 900 μL of clustering diluent to Empty Vial (which has 100 μL of the clustering stock in it from step 4) and remove syringe.
8. Attach a fresh BD syringe to the vial with the clustering stock and clustering diluent. Mix up and down using syringe. Dislodge air bubbles by moving plunger up and down when filling syringe to withdraw >150 μL into the BD syringe. Attach the dosing needle and prime syringe to 100 μL ready to dose.
9. Attach a second BD syringe to the vial with the mixed clustering stock and clustering diluent, withdraw >150 μL, attach the dosing needle, and prime the syringe to 100 μL ready to dose.
10. Prepared doses should be used within 6 hours total from thawing.

Dosing: animals (e.g., minipigs; 10 Yucatan (females 4-6 months old)) are fasted the day prior to injection. Fifteen minutes prior to anesthesia, a topical mydriatic (1.0% tropicamide HCl) is applied. Buprenorphine (0.01-0.05 mg/kg) or meloxicam (0.4 mg/kg) is administered intramuscularly (IM), as well as 0.05 mg/kg of atropine IM or 0.01 mg/kg glycopyrrolate IM. Animals are anesthetized with ketamine (10 mg/kg)/dexmedetomidine (0.05 mg/kg) IM. The area around the eye, including the eyelid, is cleaned with a 10% solution of baby shampoo using gauze, rinsed with sterile saline, topical application of 5% betadine solution, and rinsed again with sterile saline. The animal receives one drop each of 0.5% proparacaine HCl and 10.0% phenylephrine HCl topically in both eyes. Using a sterile cotton-tipped applicator soaked in 1% betadine, the conjunctival cul-de-sacs in both eyes and under the third eyelid is swabbed, followed by rinsing with sterile saline. An eyelid speculum is placed, and the test articles are administered by suprachoroidal space (SCS) injection using a 30-gauge needle approximately 900 or 1100 μm in length. For the first animal (Group 0), injections are delivered with 1 mL BD needle and MedOne 900 μm (or 1100 μm) needles are used first for injection. For the remaining animals, needles selected based on the first animal dosed are used. Injections are performed bilaterally and delivered to supra-temporal quadrant 4 mm from the limbus between 10 and 11 o'clock in the right eye and between 1 and 2 o'clock in the left eye.

Post-Injection Procedures and Recovery: following the injections, a topical antibiotic (neo-poly gramicidin or equivalent) is administered. OCT with enhanced depth imaging (EDI) is performed acquiring approximately 15 b-scans spanning the dosing site. An en-face blue autofluorescence image is then performed while the animal remains sedated and in the same position. A second OCT is done spanning the visual streak (ON placed in the top 3rd of the view), followed by a blue auto-fluorescence image. Animals receive atipamezole IM, if needed, to reverse the effects of dexmedetomidine and are allowed to recover normally from the procedure. A second drop of topical antibiotic is given following the imaging and also at 6 hours later. Prophylactic antibiotics are administered BID for 2 additional days with 6-8 hours between doses.

Parameters Measured: animals are assessed for mortality or morbidity twice daily, morning and afternoon, and body weights are acquired prior to dosing, once weekly, and at termination,

Ocular Eye Examinations (OEs): pupils are dilated for ocular examination using topical 1% tropicamide HCL (one drop in each eye 15 minutes prior to examination). Complete OEs using a slit lamp biomicroscope and indirect ophthalmoscope are used to evaluate ocular surface morphology, anterior and posterior segment at each timepoint as indicated in Table 18. The modified Hackett and McDonald ocular grading system with additional scoring parameters for the ocular posterior segment are used to grade inflammation (Hackett, R. B. and McDonald, T. O. Ophthalmic Toxicology and Assessing Ocular Irritation. Dermatoxicology, Fifth Edition. Ed. F. N. Marzulli and H. I. Maibach. Washington, D.C.: Hemisphere Publishing Corporation. 1996; 299-305 and 557-566). If high levels of ocular inflammation are noted by the veterinarian ophthalmologist after Day 8 examinations, treatment of the eyes with difluprednate BID (4-8 hours) for the remainder of the study is considered. In some cases, difluprednate treatment is required.

Tonometry: intraocular pressure (IOP) is measured in both eyes at the timepoints indicated in Table 18. Measurements are performed in non-sedated animals. The measurements are taken using a Tonovet probe (iCare Tonometer, Espoo, Finland) without use of topical anesthetic. The tip of the Tonovet probe is directed to gently contact the central cornea. The average IOP shown on the display is recorded, and three measurements are determined.

Optical Coherence Tomography (OCT): OCT with enhanced depth imaging for examination of the posterior section of the eye is performed using a Spectralis HRA OCT II (Heidelberg Engineering) immediately following injection and on Days 14, 21 and 29. Blue autofluorescence imaging is also acquired. Prior to OCT imaging on Days 14, 21, and 29, animals are fasted overnight. Animals are anesthetized with ketamine (10 mg/kg)/dexmedetomidine (0.05 mg/kg) IM and their eyes dilated using topical tropicamide HCl 1%, applied 15 minutes prior to imaging. Two sites are imaged if possible (drug deposition and visual streak). The follow-up feature is used to track changes in the injection site. Raw OCT images are delivered electronically for analysis.

Color and Cobalt Blue Fundus Imaging: accompanying the OCT imaging, animals undergo fundic imaging using the RetCam3 (Natus). Both color and cobalt blue imaging is performed. Following completion of imaging, animals receive atipamezole IM, if needed, to reverse the effects of dexmedetomidine and are allowed to recover normally from the procedure. Dose deposition site and visual streak are imaged. Fundus images is transferred electronically.

Blood Collections for Serum: serum collection for tab analysis: at baseline (prior to dosing) and just prior to euthanasia, at least 1.2 mL of whole blood is drawn from the jugular vein and deposited into non-anticoagulant tubes (1.3 mL) for serum collection from all animals. Following collection, the tubes are gently mixed by inverting the tubes 3-5 times. Blood samples are stored at room temperature for at least 30 minutes, but less than 60 minutes, prior to processing. The whole blood samples are centrifuged at 4° C. for 10 minutes at 10,000 g in a refrigerated centrifuge. Immediately after centrifugation, the clear serum is separated into two aliquots and transferred into 2 mL cryovial polypropylene tubes and placed on dry ice and stored frozen at −80° C. until used for bioanalytical analysis.

Whole Blood Collections: at least 2.0 mL of whole blood (at specific times) is drawn from the jugular vein of the animals into tubes for plasma collection. After collection, the tubes are gently mixed by inverting the tubes 5-8 times. Samples are divided into two approximately equal aliquots and transferred into separate nuclease-free tubes. Aliquots are then placed on dry ice until stored at −80° C.

Euthanasia: while remaining under sedation, animals are euthanized with an intravenous injection of an AVMA-approved barbiturate-based euthanasia agent (e.g., Euthasol). Death is ensured by auscultation.

Ocular Tissue Collections: for Groups 1-3, ocular tissue is collected immediately after euthanasia. Both eyes are enucleated, and the injection site is marked with suture. For the N=5 eyes per group (3 right eyes and two left), aqueous humor is removed via a 27 or 30-gauge syringe, weighed, and snap frozen by immersing in liquid nitrogen. The whole globe is then snap frozen in liquid nitrogen and stored at −80° C. For the N=1 eye per group (1 left eye; first animal/group), lacrimal gland is fixed in Davidson's solution (15 to 20 times greater than the volume of the tissue volume) for at least 48 hours to 72 hours at room temperature. After 72 hours of fixation, tissues are transferred and stored in 70% ethanol.

Extraocular tissues are collected from N=5 eyes per group, rinsed briefly in PBS if contaminated with blood, weighed, and snap frozen. These ocular tissues are dissected while frozen in accordance with standard operating procedure. Sclera and optic nerve are minced with a single edge razorblade prior to obtaining tissue weight. Samples are then stored at −80° C.

### List of Tissues Collected for Biodistribution or Transgene Product Level Analysis:

1. Serum, samples are split into 2 tubes per collection (2 mL polypropylene screw cap tube)
2. Whole blood samples are split into 2 tubes per collection (2 mL DNAse/RNAse free polypropylene tubes)
3. Aqueous humor (2 mL polypropylene tube)
4. Vitreous humor (5-7 mL polypropylene tube)
5. Sclera: 1 tube per collection (5-7 mL polypropylene tube)
6. Optic Nerve: 2 tubes (2 mL polypropylene screw cap tube)
7. Retina: 1 tube per collection (2 mL polypropylene screw cap tube)
8. RPE-Choroid: 1 tube per collection (2 mL polypropylene screw cap tube)
9. Iris-ciliary body: 1 tube (2 mL polypropylene screw cap tube)
10. Optic chiasm: 2 tubes (2 mL polypropylene screw cap tube)
11. Occipital lobe: 3 tubes (3) 6-7 mm biopsy punches) per collection (2 mL polypropylene tubes)
12. Frontal cortex: 3 tubes (3) 6-7 mm biopsy punches) per collection (2 mL polypropylene tubes)

Group 0: Immediately after euthanasia, both eyes are enucleated, and the injection site is marked with a tissue marker. Aqueous humor from both eyes are removed via a 27 or 30-gauge syringe, weighed, and snap frozen by immersing in liquid nitrogen. Both eyes undergo fresh ocular dissections. Vitreous humor (VH) is first collected using a 23-25-gauge needle with a 3 ml syringe. The needle is inserted 2 mm below the limbus into the central vitreous avoiding contact with the lens. VH is slowly collected and placed in a 2 ml polypropylene tube. Care is taken to not apply too much vacuum pressure. The Iris-ciliary body is then be collected. The lens is removed and the eye is cut into 4 quadrants based on the marker (superior temporal (dose site), inferior-temporal, superior nasal, and inferior nasal). Eight (8) mm punches are punched out of each quadrant, and retina, RPE/choroid, and sclera are collected separately into 2 mL polypropylene tubes. The remainder of the tissue (retina, RPE/choroid, and sclera) is then separated and collected into 2 mL polypropylene tubes. Tissue list is as follows for each eye:
1. Aqueous humor (2 mL polypropylene screw cap tube)
2. Vitreous humor (2 mL polypropylene screw cap tube)
3. Sclera: n=5 samples, 4 punches and remaining tissue (2 mL polypropylene screw cap tube for punches and 5-7 mL polypropylene tubes for remaining tissue)
4. Retina: n=5 samples, 4 punches and remaining tissue (2 mL polypropylene screw cap tubes)
5. RPE-Choroid: n=5 samples, 4 punches and remaining tissue (2 mL polypropylene screw cap tubes)
6. Iris-ciliary body 1 tube (2 mL polypropylene screw cap tube)
7. Optic chiasm: 2 tubes (2 mL polypropylene screw cap tube)
8. Occipital lobe: 3 tubes (3) 6-7 mm biopsy punches) per collection (2 mL polypropylene tubes)
9. Frontal cortex: 3 tubes (3) 6-7 mm biopsy punches) per collection (2 mL polypropylene tubes)

Non-Ocular Tissue Collections (All Groups): following enucleation, non-ocular tissues are collected and held for potential analysis. Clean collection techniques are utilized in order to avoid cross-contamination, and two samples of each tissue are taken. Samples are snap frozen and stored at −80° C. prior to analysis.

### List of Tissues for Collection:

1. Liver (6-7 mm biopsy punch, left lateral lobe) (3 samples) (2 mL polypropylene tubes)
2. Lacrimal gland main (2) (2 mL polypropylene tubes)
3. Mandibular lymph nodes (2) (2 mL polypropylene tubes)
4. Parotid lymph node (2) (2 mL polypropylene tubes)

TABLE 19
Designation of Tissues for Analysis

| Group(s) | Samples Collected | Designation of Aliquot 1 | Designation of Aliquot 2 |
|---|---|---|---|
| Groups | Left (2) Retina, | qPCR | NA (just one aliquot) |
| 1, 2, 3 | RPE/Choroid, Sclera | | |
| | Right (3) Retina, | Transgene product analysis | NA (just one aliquot) |
| | RPE/Choroid, Sclera | | |
| | Aqueous humor, lacrimal | Hold for potential analysis | NA (just one aliquot) |
| | gland, eye draining | or method development | |
| | lymph nodes | | |
| | Serum, optic nerve, iris- | qPCR | Hold for potential analysis |
| | ciliary body, optic | | or method development |
| | chiasm, occipital lobe, | | |
| | liver | | |
| | Frontal Cortex | Hold for potential analysis | Hold for potential analysis |
| | | or method development | or method development |
| | Serum | Tab Assay | Hold for potential analysis |
| | | | or method development |
| Group | Serum, sclera (all | qPCR | NA (just one aliquot) |
| 0 | punches), RPE/choroid | | |
| | (all punches), retina (all | | |
| | punches) | | |
| | Posterior segment | Hold for potential analysis | Hold for potential analysis |
| | (separate RPE/choroid, | or method development | or method development if |
| | Retina, sclera tubes), | | appropriate |
| | aqueous humor, vitreous | | |
| | humor, lacrimal gland, | | |
| | eye draining lymph nodes | | |
| | Serum, optic nerve, iris- | qPCR | Hold for potential analysis |
| | ciliary body, optic | | or method development |
| | chiasm, occipital lobe, | | |
| | liver | | |
| | Frontal Cortex | Hold for potential analysis | Hold for potential analysis |
| | | or method development | or method development |
| | Serum | Tab Assay | Hold for potential analysis |
| | | | or method development |

This study is designed to evaluate pharmacodynamics/biodistribution and toxicity of the test material following suprachoroidal injection in pigs. This study is designed to further develop a delivery platform to treat humans with ocular diseases (e.g., diseases of the uvea and retina).

### 5.14 Example 14: Suprachoroidal Formulation: Pharmacodynamic/Biodistribution, and Toxicity Study in Pigs

This example summarizes the results of Example 13. Expansion of SCS was seen in of 6 eyes administered AAV.CAG.GFP Gel Formulation, and the SCS decreased to normal by Day 15 in these eyes. On average, eyes administered AAV.CAG.GFP Gel Formulation exhibited greater transgene product (TP) in the retina (10×) compared to eyes administered AAV.CAG.GFP modified DPBS with Sucrose Formulation. Further, SCS thickness was measured after the different formulations and control were administered to the animals. It was found that the estimated thickness of the SCS increased to 2-4× of a marker (200 mm) at its widest section (about 400-800 mm) for the gel formulation treated animals. An animal having the highest predose total antibodies (Tabs) had below limit of detection GFP concentrations and was not imaged. See Tables 20 and 21.

TABLE 20
Transgene Product (GFP) protein concentration

| | | | | Mean | | | |
|---|---|---|---|---|---|---|---|
| | | | | Diluted | | Final | Final Conc. |
| Dose | Test | Animal | Matrix | Result | Dilution | Concentration | (ng of TP mg |
| Group | Article | # | Type | (pg/mL) | Factor | (pg/mL) | of Protein) |
| 1 | Modified | 89-125 | Choroid- | 53.8 | 8 | 430 | 0.0680 |
| | dPBS with | | RPE, Right | | | | |
| | Sucrose | | Eye | | | | |
| 1 | Modified | 89-114 | Choroid- | 30.7 | 8 | 245 | 0.0361 |
| | dPBS with | | RPE, Right | | | | |
| | Sucrose | | Eye | | | | |
| 1 | Modified | 90-034 | Choroid- | 168 | 8 | 1340 | 0.223 |
| | dPBS with | | RPE, Right | | | | |
| | Sucrose | | Eye | | | | |
| 3 | Clustering | 89-147 | Choroid- | 42.5 | 8 | 340 | 0.0629 |
| | | | RPE, Right | | | | |
| | | | Eye | | | | |
| 3 | Clustering | 90-013 | Choroid- | 70.1 | 8 | 561 | 0.0952 |
| | | | RPE, Right | | | | |
| | | | Eye | | | | |
| 3 | Clustering | 90-047 | Choroid- | 62.0 | 8 | 496 | 0.0859 |
| | | | RPE, Right | | | | |
| | | | Eye | | | | |
| 1 | Modified | 89-125 | Sclera, | 120 | 8 | 961 | 0.498 |
| | dPBS with | | Right Eye | | | | |
| | Sucrose | | | | | | |
| 1 | Modified | 89-114 | Sclera, | 249 | 8 | 1990 | 0.776 |
| | dPBS with | | Right Eye | | | | |
| | Sucrose | | | | | | |
| 1 | Modified | 90-034 | Sclera, | 312 | 8 | 2500 | 0.970 |
| | dPBS with | | Right Eye | | | | |
| | Sucrose | | | | | | |
| 3 | Clustering | 89-147 | Sclera, | 80.0 | 8 | 640 | 0.218 |
| | | | Right Eye | | | | |
| 3 | Clustering | 90-013 | Sclera, | 105 | 8 | 841 | 0.399 |
| | | | Right Eye | | | | |
| 3 | Clustering | 90-047 | Sclera, | 223 | 8 | 1790 | 0.706 |
| | | | Right Eye | | | | |
| 1 | Modified | 89-125 | Retina, | Avg. | Avg. | 40.7 | 0.00988 |
| | dPBS with | | Right Eye | of 2 | of 2 | | |
| | Sucrose | | | | | | |
| 1 | Modified | 89-114 | Retina, | 27.4 | 1 | 27.4 | 0.00596 |
| | dPBS with | | Right Eye | | | | |
| | Sucrose | | | | | | |
| 1 | Modified | 90-034 | Retina, | Avg. | Avg. | 42.2 | 0.00933 |
| | dPBS with | | Right Eye | of 2 | of 2 | | |
| | Sucrose | | | | | | |
| 3 | Clustering | 89-147 | Retina, | Avg. | Avg. | 38.6 | 0.00664 |
| | | | Right Eye | of 3 | of 3 | | |
| 3 | Clustering | 90-013 | Retina, | Avg. | Avg. | 43.7 | 0.00761 |
| | | | Right Eye | of 2 | of 2 | | |
| 3 | Clustering | 90-047 | Retina, | Avg. | Avg. | 56.0 | 0.00938 |
| | | | Right Eye | of 2 | of 2 | | |

TABLE 21
Control measurement for total protein concentration in each eye tissue

| | | | | | | | Total Eye |
|---|---|---|---|---|---|---|---|
| | | | | | | | Tissue |
| | | | | | | | Protein − |
| | | | | Tissue | Mean | | Final |
| Dose | Test | Animal | Matrix | Mass | Result | Dilution | Concentration |
| Group | Article | # | Type | (mg) | (mg/mL) | Factor | (mg/mL) |
| 1 | Modified | 89-125 | Choroid- | 48.6 | 0.632 | 10 | 6.32 |
| | dPBS with | | RPE, Right | | | | |
| | Sucrose | | Eye | | | | |
| 1 | Modified | 89-114 | Choroid- | 58.8 | 0.678 | 10 | 6.78 |
| | dPBS with | | RPE, Right | | | | |
| | Sucrose | | Eye | | | | |
| 1 | Modified | 90-034 | Choroid- | 56.8 | 0.601 | 10 | 6.01 |
| | dPBS with | | RPE, Right | | | | |
| | Sucrose | | Eye | | | | |
| 3 | Clustering | 89-147 | Choroid- | 79.2 | 0.541 | 10 | 5.41 |
| | | | RPE, Right | | | | |
| | | | Eye | | | | |
| 3 | Clustering | 90-013 | Choroid- | 71.3 | 0.589 | 10 | 5.89 |
| | | | RPE, Right | | | | |
| | | | Eye | | | | |
| 3 | Clustering | 90-047 | Choroid- | 55.0 | 0.578 | 10 | 5.78 |
| | | | RPE, Right | | | | |
| | | | Eye | | | | |
| 1 | Modified | 89-125 | Sclera, | 384 | 0.193 | 10 | 1.93 |
| | dPBS with | | Right Eye | | | | |
| | Sucrose | | | | | | |
| 1 | Modified | 89-114 | Sclera, | 465 | 0.257 | 10 | 2.57 |
| | dPBS with | | Right Eye | | | | |
| | Sucrose | | | | | | |
| 1 | Modified | 90-034 | Sclera, | 392 | 0.258 | 10 | 2.58 |
| | dPBS with | | Right Eye | | | | |
| | Sucrose | | | | | | |
| 3 | Clustering | 89-147 | Sclera, | 512 | 0.293 | 10 | 2.93 |
| | | | Right Eye | | | | |
| 3 | Clustering | 90-013 | Sclera, | 466 | 0.211 | 10 | 2.11 |
| | | | Right Eye | | | | |
| 3 | Clustering | 90-047 | Sclera, | 400 | 0.254 | 10 | 2.54 |
| | | | Right Eye | | | | |
| 1 | Modified | 89-125 | Retina, | 71.4 | 0.411 | 10 | 4.11 |
| | dPBS with | | Right Eye | | | | |
| | Sucrose | | | | | | |
| 1 | Modified | 89-114 | Retina, | 64.4 | 0.460 | 10 | 4.60 |
| | dPBS with | | Right Eye | | | | |
| | Sucrose | | | | | | |
| 1 | Modified | 90-034 | Retina, | 99.8 | 0.452 | 10 | 4.52 |
| | dPBS with | | Right Eye | | | | |
| | Sucrose | | | | | | |
| 3 | Clustering | 89-147 | Retina, | 56.9 | 0.581 | 10 | 5.81 |
| | | | Right Eye | | | | |
| 3 | Clustering | 90-013 | Retina, | 75.8 | 0.574 | 10 | 5.74 |
| | | | Right Eye | | | | |
| 3 | Clustering | 90-047 | Retina, | 84.2 | 0.597 | 10 | 5.97 |
| | | | Right Eye | | | | |

### 5.15. Example 15: Suprachoroidal Formulation: Exploratory Study in Pigs

This example relates to the evaluation of pharmacokinetic/pharmacodynamic (PK/PD) and biodistribution different formulations of a test AAV expressing a secreted transgene product (e.g. IgG or Fab) in animals (e.g., minipigs) after a single suprachoroidal injection. This example further aims to characterize PK/PD of full-length and fragment antibody in the eye and to assess TP concentrations in ocular compartments. The study is performed analogously to Example 13, having the study design as illustrated in Table 22.

TABLE 22
Study Outline for Lanadelumab Dosing

| | | | | | |
|---|---|---|---|---|---|
| Study | | Dose | | # of | |
| Groups | TM | (GC/kg) | Route | animals | Necropsy |

Cohort 1

| | | | | | |
|---|---|---|---|---|---|
| 1 | AAV8.CAG.Lan.IgG | Low | SR | 2 | SR = 8 week; SCS = 4 week |
| 2 | AAV8.CAG.Lan.IgG | High | SCS | 2 | Protein: 1 eye |
| | | | | | RNA: 1 eye |
| | | | | | DNA: 1 eye |
| | | | | | Histology: 1 eye |

Cohort 2

| | | | | | |
|---|---|---|---|---|---|
| 3 | AAV8.CAG.Lan.Fab | Low | SR | 2 | SR = 8 week; SCS = 4 week |
| 4 | AAV8.CAG.Lan.Fab | High | SCS | 2 | Protein: 1 eye |
| | | | | | RNA: 1 eye |
| | | | | | DNA: 1 eye |
| | | | | | Histology: 1 eye |

Cohort 3 (dependent on data from Cohort 1)

| | | | | | |
|---|---|---|---|---|---|
| 1 | AAV8.CAG.Lan.IgG | Low | SR | 3 | SR = 8 week; SCS = 4 week |
| 2 | AAV8.CAG.Lan.IgG | High | SCS | 3 | Protein: 2 eyes |
| | | | | | RNA: 1 eye |
| | | | | | DNA: 2 eyes |
| | | | | | Histology: 1 eye |

Cohort 4 (dependent on data from Cohort 2)

| | | | | | |
|---|---|---|---|---|---|
| 3 | AAV8.CAG.Lan.Fab | Low | SR | 3 | SR = 8 week; SCS = 4 week |
| 4 | AAV8.CAG.Lan.Fab | High | SCS | 3 | Protein: 2 eyes |
| | | | | | RNA: 1 eye |
| | | | | | DNA: 2 eyes |
| | | | | | Histology: 1 eye |

Cohort 5 (dependent on data from Cohort 1)

| | | | | | |
|---|---|---|---|---|---|
| 1 | AAV8.CAG.Lan.IgG | Low | SR | 2 | SR = 4 week |
| | | | | | Protein: 2 eyes |
| | | | | | RNA: 1 eye |
| | | | | | DNA: 2 eyes |
| | | | | | Histology: 1 eye |

Lan.IgG: Lanadelumab full-length antibody
Lan.Fab: Landelumab Fab

Administration of formulated AAV.Lanadelumab is administered either subretinally or suprachoroidally, as indicated in the above table. Low dose SCS: 3×10¹¹ GC/eye; High dose SCS: 5×10¹¹-10×10¹¹ GC/eye or 7×10¹¹ GC/eye. Low dose SR: 1×10¹⁰-3×10¹⁰ GC/eye; High dose SR: 3×10¹⁰-7×10¹⁰ GC/eye. Dose volume for both: 100 uL as single injection. Clinical observations include twice daily animal room checks, weekly body weight, and food consumption (qualitative only).

### Observations/Intervals:

Ophthalmic Exams/IOPs Predose (1 week after AH humor collection): ˜Day 3, and Weeks 1, 2, 4, 6, and 8 postdose On days of Aqueous humor tap, OEs are conducted prior to tap.
Aqueous Humor Collection: Predose, and ˜Weeks 2, 4, 6, 8 postdose
Color Imaging/Fluorescein Angiography: Predose (all), Day 1 after dosing (SR animals only) and ˜Weeks 2, 4, 8 postdose. Area centralis, temporal and nasal fields. For SCS, attempts are made to image the temporal superior quadrant at all intervals (dose delivery site)
Optical Coherence Tomography (OCT): Predose (all), ˜Weeks 4 and 8 postdose. Area centralis, temporal and nasal fields. For SCS, attempts are made to image the temporal superior quadrant at all intervals (dose delivery site).
Serum Collections: TAB Assay: Predose, ˜Weeks 2, 4, 6, 8 postdose (serum). TP and ATPA: Predose, ˜Weeks 2, 4, 6, 8 postdose (serum).
Whole blood samples for AAV vector DNA analysis (qPCR): Predose, ˜Weeks 2, 4, 8 postdose (serum).
   Upon termination (at 4 or 8 weeks), the following tissues are collected:
Aqueous humor
Vitreous humor
Optic Nerve
Iris-ciliary body
Optic chiasm
Occipital lobe
Frontal cortex
Cornea
Lens

Samples are collected fresh; for posterior segment 4 8-mm punches and remaining posterior eye cup are collected and separated into retina, RPE/choroid, and sclera.

Non-ocular tissues are also collected and examined:
Liver (left lateral lobe; 3 samples)
Lacrimal gland main (2)
Mandibular lymph nodes (2)
Parotid lymph node (2)

### 5.16. Example 16: Full AAV Particle Aggregates in Low Ionic Strength Buffer, Allowing Methods to Improve AAV Full Capsid Percentage by Inducing Selective Reversible Aggregation of Full AAV Particles

AAVs transduce cells by binding to cell receptors and deliver the transgene to the cell nucleus to achieve protein expression (D. Wang, P. W. L. Tai and G. Gao, 2019. Adeno-associated virus vector as a platform for gene therapy delivery. Nature Reviews Drug Discovery 18, 358-378). The presence of empty capsids in AAV drug substances has been a challenge to the field (R. Rieser, J. Koch, G. Faccioli, K. Richter, T. Menzen, M. Biel, G. Winter and S. Michalakis, 2021. Comparison of Different Liquid Chromatography-Based Purification Strategies for Adeno-Associated Virus Vectors. Pharmaceutics 13). AAV empty capsids possess the same viral capsid proteins as the full capsid, but have no therapeutic genome encapsidated by such capsid, and as such they potentially compete for cell receptor binding sites thus decreasing in vivo gene delivery efficacy (Gao, 2014. Empty Virions In AAV8 Vector Preparations Reduce Transduction Efficiency And May Cause Total Viral Particle Dose-Limiting Side-Effects. Mol Ther Methods Clin Dev 1, 20139). In addition, empty capsids expose the immune system to unnecessary foreign proteins. Currently empty capsids are removed by either density gradient centrifugation, which is not readily scalable, or by ion exchange chromatography, which lacks resolution to fully remove empty capsids with high full capsids yield (B. Adams, H. Bak and A. D. Tustian, 2020. Moving from the bench towards a large scale, industrial platform process for adeno-associated viral vector purification. Biotechnol Bioeng 117, 3199-3211; T. Kimura, B. Ferran, Y. Tsukahara, Q. Shang, S. Desai, A. Fedoce, D. R. Pimentel, I. Luptak, T. Adachi, Y. Ido, R. Matsui and M. M. Bachschmid, 2019. Production of adeno-associated virus vectors for in vitro and in vivo applications. Sci Rep 9, 13601).

### Methods:

Size Distribution by Dynamic light Scattering: Dynamic Light Scattering (DLS) was performed on a DynaProIII (Wyatt Technology Corporation, Goleta, CA) using 384 well plates (3540, Corning, Corning, NY) with a 30 μL sample volume. Ten acquisitions, each for 10 s, were collected per replicate and three replicate measurements were made per sample. The average and SD of the cumulants diameter was reported.

Colloidal Stability Screening: to adjust the ionic strength for colloidal stability measurements, the sodium chloride level was varied with all other excipients held at their target level. The other buffer and electrolytes that were held constant contributed 13 mM of ionic strength in an intrathecal formulation (see F4 below) and 29 mM of ionic strength in modified DPBS with sucrose formulation (see F3 below).

Ionic strength is defined as half of the sum of concentration of ions (ci) in solution weighted by their charge (zᵢ) squared:

$$I = \frac{1}{2} {{\sum }_{i = 1}}^{n} {c}_{i} {z}_{i}^{2}$$

Spectrophotometry (OD) Method: samples were analyzed with the Cary 60 spectrophotometer using a 1 cm pathlength in 50 μL cuvettes. Scans were performed with data collected from 400 nm to 200 nm, with a data interval of 1 nm and scan rate of 0.1 second/wavelength.

Reversibility of Aggregation: to assess reversibility of aggregation, five freeze-thaw cycles were applied to AAV9 formulated in F4 at various ionic strengths and aggregation assessed by DLS. After aggregation was induced by freeze-thaw at low ionic strength, a small volume of concentrated sodium chloride was spiked back to bring the total back to a level of 150 mM and aggregation was reassessed by DLS.

### Formulations:

F3 (modified DPBS with sucrose and 0.001% poloxamer 188): 100 mM sodium chloride, 2.70 mM potassium chloride, 8.10 mM sodium phosphate dibasic anhydrous, 1.47 mM potassium phosphate monobasic, 117 mM sucrose, 0.001% w/v poloxamer 188, pH 7.4. For F3, high-purity sucrose was used (S-124-2-MC, Pfanstiehl, Waukegan, IL).

F4 (intrathecal formulation): 150 mM sodium chloride, 1.20 mM magnesium chloride 6-hydrate, 0.201 mM sodium phosphate monobasic monohydrate, 0.803 mM sodium phosphate dibasic anhydrous, 3.00 mM potassium chloride, 1.40 mM calcium chloride dihydrate, 4.40 mM dextrose anhydrous, 0.001% w/v poloxamer 188, pH 7.2.

### Results:

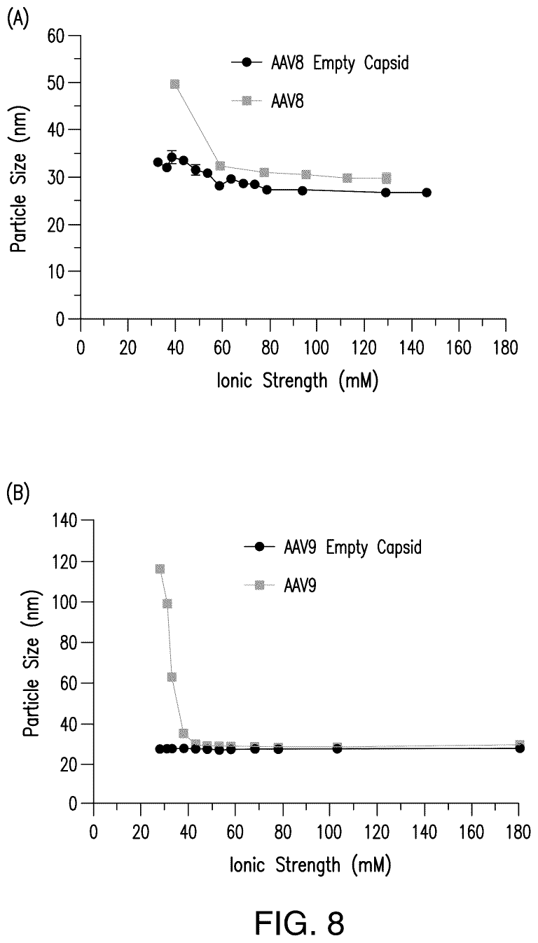

As shown in FIGS. 8A and 8B, when the ionic strength of the formulation buffer was decreased to below approximately 50 mM, the colloidal stability of AAV8 and AAV9 was decreased, resulting in self-association and an increase in particle size (detected aggregates of AAV vectors). In contrast, data showed that empty capsids did not aggregate at low ionic strength conditions and remained soluble and monomeric in the 30 to 50 mM ionic strength range (Refer to solid circle in FIGS. 8A and 8B).

To assess the reversibility of aggregation, aggregates of AAV9.transgene (carrying a transgene encoding an enzyme) formulated in F4 was induced at various ionic strengths, followed by five freeze-thaw cycles and spiking back to a level of 150 mM NaCl to assess the robustness of the aggregation and reversal process. Data showed that the self-association was largely reversible as shown in Table 23. After inducing aggregation in the low ionic strength buffers (16-36 mM), the average AAV particle size returned to its monomeric size when the ionic strength in the buffer was increased to 167 mM.

TABLE 23
Reversibility of Aggregation of AAV9 in F4 after 5 Freeze-Thaw Cycles
Performed at Low Salt Followed with Spiking to 150 mM Salt

| | | Diameter after 5 |
|---|---|---|
| Initial Ionic | Initial | F/T Cycles and Salt |
| Strength | Diameter | Spike to 150 mM |
| (mM) | (nm) | (nm) |
| 16 | 101.5 | 42.8 |
| 19 | 118.9 | 34.0 |
| 21 | 115.5 | 37.9 |
| 26 | 86.9 | 31.2 |
| 31 | 70.3 | 31.4 |
| 36 | 43.1 | 29.3 |
| 46 | 30.1 | 28.6 |
| 91 | 28.4 | 28.8 |
| 167 | 28.5 | 28.5 |

This selective aggregation of full capsids was identified at low ionic strength conditions where empty capsids remain soluble, and thus could be used as the basis of a purification step to remove empty capsids. Prior art have cited aggregation of AAVs as a cause of product losses to be avoided. However, a key aspect of the current disclosure is that the aggregation can be reversed and therefore used as a process purification step. This could be designed by allowing aggregation of full capsids to occur and then capturing them on a filter or centrifuging the sample to pellet the full capsids. The full capsid is then recovered by reversing the aggregation in the presence of a high ionic strength buffer, and the soluble empty capsids can be discarded.

In another design, the aggregation technique can be utilized at bedside, or during the preparation of AAV vectors for administration. Following induction of clustering of AAV particles in solution prior to suprachoroidal administration, then a further step of centrifuging the solution to recover a concentrated pellet of full AAV particles can be employed. The empty AAV particles are removed or decanted. The concentrated pellet of full AAV particles is brought to dosing volume with low ionic strength diluent to maintain aggregated AAV particles for further administration to the subject.

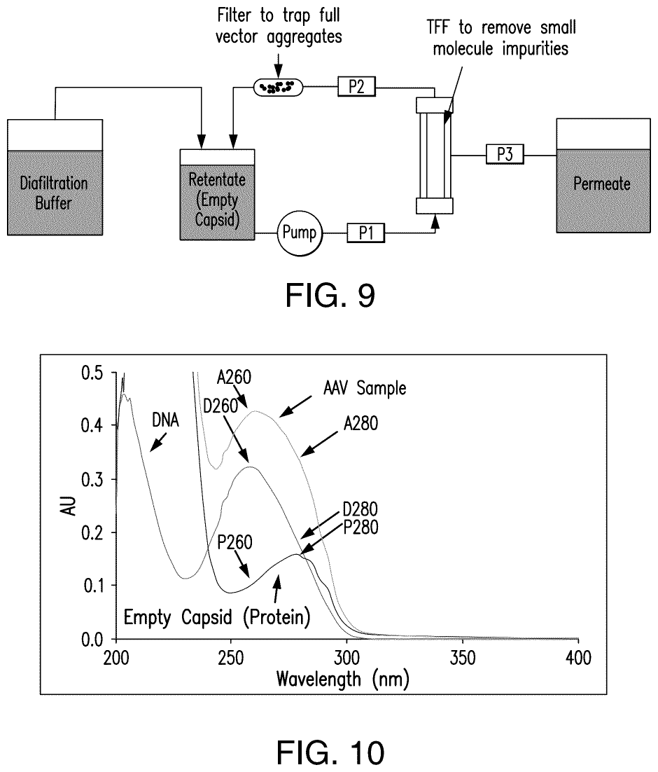

In some cases, for larger scale processing, diafiltration using a Hollow Fiber-Tangential Flow Filtration (HF-TFF) system with an in-line filter to capture aggregated particles of full AAV is used. Other approaches, such as spiking with low ionic strength to induce aggregation of full AAV molecules and collecting particles of full AAV by centrifugation can also be employed. In the diafiltration example (FIG. 9), diafiltration with a formulation buffer with a very low ionic strength (1 mM phosphate buffer and 0.001% Poloxamer 188) can be used to induce full AAV capsid aggregation on the filter during circulation. The empty capsids are recirculated back to the retentate vessel while the full capsids gradually build up as aggregates on the porous materials in the filter. The decrease of full AAV capsids in the retentate vessel can be monitored by the ratio between 260 and 280 nm with a UV-Vis spectrometer (FIG. 10) to determine the optimal buffer exchange needed. An increase of absorption at 280 nm and a decrease at 260 nm in the retentate occurs as the full AAV capsids aggregate on the filter. Once a sufficiently high collection of full AAV capsids on the filter is achieved, the empty capsids retentate vessel are removed. The full AAV capsids are recovered into a new retentate vessel by changing to a high ionic strength buffer (>150 mM) that reverses the aggregation and dissociates AAV aggregates to monomers. This purification method can be easily scaled and eliminate labor-intensive and costly purification methods such as anion exchange chromatography.

### 5.17 Example 17: Suprachoroidal Formulation: Pharmacodynamic/Biodistribution Study in Minipigs

This example evaluated the pharmacodynamic and biodistribution of clustering formulation of AAV.GFP in minipigs after a single suprachoroidal injection. Methods and materials used in this example were substantially the same as the methods and materials disclosed in Example 13 of the present disclosure.

This example primarily evaluated whether the clustering formulation was associated with increased protein levels and DNA in retina or RPE/choroid compared to the sucrose formulation. This example further evaluated the in vivo imaging techniques for monitoring the presence of formulation in SCS space, the in vivo imaging techniques for expression monitoring, and the distribution of transgene product (TP), vector DNA and RNA. Minipigs were chosen for this example because of the similarity to humans in eye size, sclera, blood supply, and retina of humans. Study design of this example is shown in Table 24.

TABLE 24
Group Assignment and Dose Levels

| | | Number of | Dose Level | |
|---|---|---|---|---|
| Group | Vector/Formulation | Animals | (GC/Eye) | End Points |
| 0 | AAV.CAG.GFP | 1 | 3 × 10¹¹ | To test needle length and compared |
| | Modified DPBS with | | | DNA with different tissue collection |
| | Sucrose | | | Fundus Image, OCT with AF and |
| 1 | AAV.CAG.GFP | 3 + 3 | 3 × 10¹¹ | enhanced depth imaging, OE |
| (a + b) | Modified DPBS with | | | Tissues for TP - right eyes |
| | Sucrose | | | (Completed) |
| 2 | AAV.CAG.GFP | 3 | 3 × 10¹¹ | DNA/RNA - 2 left eyes (DNA for |
| | Clustering | | | initial animals only) |
| | Formulation | | | GFP IHC and ISH - 1 left eye |

Expansion of SCS space was mild in about half of the eyes administered with sucrose and clustering formulations,

Vector DNA biodistribution (BD) data in minipigs (cohort a) is shown in Table 26 below. Each datapoint in Table 25 represented one animal (OS) with measurements done at day 29. Whole tissue was collected. All liver samples showed BLQ (BLQ=50 copies/r×n).

TABLE 25
Vector DNA Biodistribution.

| | AAV.CAG.GFP | | | AAV.CAG.GFP | |
|---|---|---|---|---|---|
| | Modified DPBS with | | | Clustering Formulation | |
| | Sucrose Vector DNA | | | Vector DNA | |
| | (GC/μg DNA) | | | (GC/μg DNA) | |
| Retina | 1790 | 50 | 50 | 50 | 50 |
| Choroid | 408 | 1750 | 5690 | 1810 | 4740 |
| Sclera | 128000 | 119000 | 481000 | 75600 | 125000 |

The BD pattern of vector DNA in different tissues was sclera>RPE/choroid>retina, which was consistent with the BD pattern in cynomolgus monkeys shown in Example 12. Moreover, the BD was comparable between the sucrose and the clustering formulations for vector DNA. In cynomolgus monkey SCS studies, the clustering formulations had higher retina and choroid BD than sucrose formulation. In general, the BD in cynomolgus monkeys was higher than in minipigs, except for sucrose formulation in retina.

The transgene product (GFP) concentrations in tissues were also measured (cohorts a and b) and shown in Table 26. OD was collected for TP quantification. BLQ was 18.8 μg/ml/mg total protein. Data distribution and averages from cohorts a and b were comparable.

TABLE 26
Transgene Product (GFP) Concentrations in Tissues

| | AAV.CAG.GFP | | | | | | AAV.CAG.GFP | | |
|---|---|---|---|---|---|---|---|---|---|
| | Modified DPBS with Sucrose | | | | | | Clustering Formulation | | |
| | GFP concentration (ng/mg protein) | | | | | | GFP concentration (ng/mg protein) | | |
| Retina | 0.00988 | 0.00596 | 0.00933 | 0.0034 | 0.0029 | 0.0029 | 0.00664 | 0.00761 | 0.00938 |
| Choroid | 0.068 | 0.036 | 0.223 | 0.258 | 0.16 | 0.0037 | 0.0629 | 0.0952 | 0.0859 |
| Sclera | 0.498 | 0.776 | 0.97 | 0.879 | 1.143 | 0.137 | 0.218 | 0.399 | 0.706 |

On average, the clustering formulation resulted in less transgene product (TP) expression in sclera with an increase in TP expression in retina 29 days following rAAV SCS injection, as compared to the control formulation that does not induce clustering of rAAV.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, and wherein the pharmaceutical composition has an amount of viral vector aggregation such that when administered to an eye of a pig:
a. the clearance time of the pharmaceutical composition is between about 5 days and about 15 days; and
b. the thickness of the SCS at the site of injection is between about 400 μm and about 800 μm at a time within one hour of administration; and
c. the circumferential spread of the pharmaceutical composition from the site of injection is about one-eighth or less of a surface of the choroid at a time within about one hour of administration.

2. The pharmaceutical composition of claim 1, wherein the pharmaceutical composition comprises an ionic strength of at most about 200 mM prior to suprachoroidal administration.

3. The pharmaceutical composition of claim 1, wherein the pharmaceutical composition comprises at least about 3% aggregated recombinant AAV prior to suprachoroidal administration.

4. The pharmaceutical composition of any one of claims 1-3, wherein the clearance time after suprachoroidal administration of the pharmaceutical composition is equal to or greater than the clearance time after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

5. The pharmaceutical composition of any one of claims 1-3, wherein a circumferential spread after suprachoroidal administration of the pharmaceutical composition is smaller as compared to a circumferential spread after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

6. The pharmaceutical composition of any one of claims 1-3, wherein a thickness at a site of injection after suprachoroidal administration of the pharmaceutical composition is equal to or higher as compared to a thickness at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

7. The pharmaceutical composition of any one of claims 1-3, wherein an expression level of the transgene is detected in the eye for a longer period of time after suprachoroidal administration of the pharmaceutical composition as compared to a period of time that an expression level of the transgene is detected in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

8. The pharmaceutical composition of any one of claims 1-3, wherein the concentration of the transgene product in the eye after suprachoroidal administration of the pharmaceutical composition is equal to or higher as compared to the concentration of the transgene product in the eye after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition;
optionally wherein the concentration of the transgene product in the back of the eye (e.g., retina) after suprachoroidal administration of the pharmaceutical composition is equal to or higher as compared to the concentration of the transgene product in the back of the eye after suprachoroidal administration of a reference pharmaceutical composition, and/or the concentration of the transgene product in the outer layer of the eye (e.g., sclera) after suprachoroidal administration of the pharmaceutical composition is lower than the concentration of the transgene product in the outer layer of the eye after suprachoroidal administration of a reference pharmaceutical composition.

9. The pharmaceutical composition of any one of claims 1-3, wherein the rate of transduction at a site of injection after suprachoroidal administration is equal to or higher as compared to the rate of transduction at a site of injection after suprachoroidal administration of a reference pharmaceutical composition, wherein the reference pharmaceutical composition comprises the recombinant AAV comprising the expression cassette encoding the transgene, wherein an amount of the recombinant AAV genome copies is the same when the pharmaceutical composition or the reference pharmaceutical composition is administered to the suprachoroidal space, and wherein the pharmaceutical composition has lower ionic strength and/or a higher level of aggregated recombinant AAV than the reference pharmaceutical composition.

10. The pharmaceutical composition of any one of claims 2-9, wherein the transgene is not an anti-human vascular endothelial growth factor (anti-VEGF) antibody.

11. The pharmaceutical composition of any one of claims 2-10, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

12. The pharmaceutical composition of any one of claims 1-11, wherein the recombinant AAV is AAV8.

13. The pharmaceutical composition of any one of claims 1-11, wherein the recombinant AAV is AAV9.

14. The pharmaceutical composition of any one of claims 1-13, wherein the pharmaceutical composition has an ionic strength of about or at most about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM.

15. The pharmaceutical composition of any one of claims 1-14, wherein the pharmaceutical composition comprises at least about or about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% aggregated recombinant AAV.

16. The pharmaceutical composition of any one of claims 1-15, wherein the pharmaceutical composition has an ionic strength of about or of at most about 40 mM.

17. The pharmaceutical composition of any one of claims 1-15, wherein the pharmaceutical composition has an ionic strength of about or of at most about 135 mM.

18. The pharmaceutical composition of any one of claims 1-15, wherein the pharmaceutical composition has an ionic strength of about or of at most about 20 mM.

19. The pharmaceutical composition of any one of claims 1-18, wherein the pharmaceutical composition has an average recombinant AAV diameter of about or at least about: 28 nm, 29 nm, 30 nm, 31 nm, 32 nm, 33 nm, 34 nm, 35 nm, 36 nm, 37 nm, 38 nm, 39 nm, 40 nm, 45 nm, 50 nm, 55 nm, 60 nm, 65 nm, 70 nm, 75 nm, 80 nm, 85 nm, 90 nm, 95 nm, or about or at least about 100 nm as measured by dynamic light scattering.

20. The pharmaceutical composition of any one of claims 4-19, wherein the pharmaceutical composition has an average recombinant AAV diameter that is at least 2 times higher, at least 3 times higher, at least 4 times higher, at least 5 times higher, at least 6 times higher, at least 7 times higher, at least 8 times higher, at least 9 times higher, at least 10 times higher, at least 15 times higher, at least 20 times higher, at least 50 times higher, at least 100 times higher, at least 5% higher, at least 10% higher, at least 15% higher, at least 20% higher, at least 25% higher, at least 30% higher, at least 35% higher, at least 40%, at least 45% higher, at least 50% higher, at least 55% higher, at least 60% higher, at least 65% higher, at least 70% higher, at least 75% higher, at least 80% higher, at least 85% higher, at least 90% higher, at least 95% higher, at least 100% higher, at least 150% higher, or at least 200% higher, at least 250% higher, or at least 300%, at least 400% higher, or at least 500% higher than an average recombinant AAV diameter in the reference pharmaceutical composition.

21. The pharmaceutical composition of any one of claims 5 and 10-20, wherein the circumferential spread after suprachoroidal administration of the pharmaceutical composition is smaller by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

22. The pharmaceutical composition of any one of claims 4 and 10-21, wherein the clearance time after suprachoroidal administration of the pharmaceutical composition is greater by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or at least 500%.

23. The pharmaceutical composition of any one of claims 1-22, wherein the clearance time after suprachoroidal administration of the pharmaceutical composition is of about 6 days to about 15 days, about 7 days to about 15 days, about 8 days to about 15 days, about 9 days to about 15 days, about 10 days to about 15 days, about 11 days to about 15 days, about 12 days to about 15 days, about 13 days to about 15 days, about 14 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, or about 5 days to about 6 days.

24. The pharmaceutical composition of any one of claims 1-23, wherein the clearance time after suprachoroidal administration of the pharmaceutical composition is not prior to about 5 days, 6 days, 7 days, 8 days, 9 days, 10 days, 11 days, 12 days, 13 days, 14 days, or 15 days.

25. The pharmaceutical composition of any one of claims 4-24, wherein the clearance time after suprachoroidal administration of the reference pharmaceutical composition is of at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, 11 days, 12 days, 13 days, or 14 days.

26. The pharmaceutical composition of any one of claims 1-25, wherein the clearance time is from the SCS or from the eye.

27. The pharmaceutical composition of any one of claims 1-26, wherein the clearance time is the time required for the pharmaceutical composition and/or the recombinant adeno-associated virus (AAV) vector to not be detectable in the SCS by any standard method.

28. The pharmaceutical composition of any one of claims 1-26, wherein the clearance time when the pharmaceutical composition and/or the recombinant adeno-associated virus (AAV) vector is present in the SCS in an amount that is at most about 2% or at most about 5% of the amount detectable by any standard method.

29. The pharmaceutical composition of any one of claims 1-26, wherein the clearance time is the amount of time required following injection for the thickness at the site of injection to decrease to about 500 nm or less, about 200 nm or less, about 100 nm or less, about 50 nm or less, about 25 nm or less, about 10 nm or less, or is undetectable.

30. The pharmaceutical composition of any one of claims 1-26, wherein the clearance time is the amount of time required following injection for the pharmaceutical composition to spread circumferentially from the site of injection to cover about one-sixteenth or more, about one-eighth or more, about one-fourth or more, about one-half or more, about three-fourths or more, or all of the circumference of the choroid of the eye.

31. The pharmaceutical composition of any one of claims 6 and 10-30, wherein the thickness at the site of injection after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

32. The pharmaceutical composition of any one of claims 1-31, wherein the thickness at the site of injection after suprachoroidal administration of the pharmaceutical composition is about 500 μm to about 3.0 mm, 750 μm to about 2.8 mm, about 750 μm to about 2.5 mm, about 750 μm to about 2 mm, or about 1 mm to about 2 mm.

33. The pharmaceutical composition of any one of claims 1-32, wherein the thickness at the site of injection after suprachoroidal administration of the pharmaceutical composition is of at least about 50 μm, 100 μm, 200 μm, 300 μm, 400 μm, 500 μm, 600 μm, or 700 μm, 800 μm, 900 μm, 1000 μm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

34. The pharmaceutical composition of any one of claims 6 and 10-33, wherein the thickness at the site of injection after suprachoroidal administration of the reference pharmaceutical composition is of at most about 1 nm, 5 nm, 10 nm, 25 nm, 50 nm, 100 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 50 μm, 100 μm, 200 μm, 300 μm, 400 μm, 500 μm, 600 μm, 700 μm, 800 μm, 900 μm, or 1000 μm.

35. The pharmaceutical composition of any one of claims 1-34, wherein the thickness of the SCS at the site of injection is about 400 μm to about 700 μm, about 400 μm to about 600 μm, about 400 μm to about 500 μm, about 500 μm to about 800 μm, about 600 μm to about 800 μm, 700 μm to about 800 μm at a time within one hour of administration.

36. The pharmaceutical composition of claim 35, wherein the time within one hour of administration is within about 5 minutes of administration, within about 10 minutes of administration, within about 15 minutes of administration, within about 20 minutes of administration, within about 30 minutes of administration, within about 45 minutes of administration, or within about 60 minutes of administration.

37. The pharmaceutical composition of any one of claims 1-36, wherein the thickness at the site of injection after suprachoroidal administration of the pharmaceutical composition persists for at least two hours, at least three hours, at least four hours, at least five hours, at least six hours, at least seven hours, at least eight hours, at least ten hours, at least twelve hours, at least eighteen hours, at least twenty-four hours, at least two days, at least three days, at least five days, at least ten days, at least twenty-one days, at least one month, at least six weeks, at least two months, at least three months, at least 4 months, at least 5 months, at least 6 months, at least 9 months, at least one year, at least three years, or at least five years.

38. The pharmaceutical composition of any one of claims 1-36, wherein the circumferential spread of the pharmaceutical composition from the site of injection is about one-eighth or less of a surface of the choroid at a time within about 5 minutes of administration, within about 10 minutes of administration, within about 15 minutes of administration, within about 20 minutes of administration, within about 30 minutes of administration, within about 45 minutes of administration, or within about 60 minutes of administration.

39. The pharmaceutical composition of claim 38, wherein the circumferential spread from the site of injection is about one-sixteenth or less of a surface of the choroid.

40. The pharmaceutical composition of any one of claims 8 and 10-39, wherein the concentration of the transgene in the eye after suprachoroidal administration of the pharmaceutical composition is higher by at least 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

41. The pharmaceutical composition of any one of claims 7 and 10-40, wherein the longer period of time after suprachoroidal administration of the pharmaceutical composition is longer by at least 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

42. The pharmaceutical composition of any one of claims 1-41, wherein the transgene is detected in the eye after suprachoroidal administration of the pharmaceutical composition for at least about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, 100 days, 120 days, 140 days, 160 days, 180 days, 200 days, 220 days, 240 days, 260 days, 280 days, 300 days, 320 days, 340 days, 360 days, 380 days, or 400 days.

43. The pharmaceutical composition of any one of claims 4-42, wherein the transgene is detected in the eye after suprachoroidal administration of the reference pharmaceutical composition for at most about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 1 day, 2 days 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 23 days, 25 days, 27 days, 30 days, 35 days, 40 days, 50 days, 55 days, 60 days, 65 days, 70 days, 75 days, 80 days, 85 days, 90 days, 95 days, or 100 days, 120 days, 140 days, 160 days, 180 days, 200 days, 220 days, 240 days, 260 days, 280 days, 300 days, 320 days, 340 days, 360 days, 380 days, or 400 days after.

44. The pharmaceutical composition of any one of claims 9 and 10-43, wherein the rate of transduction at the site of injection after suprachoroidal administration of the pharmaceutical composition is higher by at least about 2 times, at least 3 times, at least 4 times, at least 5 times, at least 6 times, at least 7 times, at least 8 times, at least 9 times, at least 10 times, at least 15 times, at least 20 times, at least 50 times, at least 100 times, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, or at least 200%, at least 250%, or at least 300%, at least 400%, or by at least 500%.

45. The pharmaceutical composition of any one of claims 2-44, wherein the recombinant AAV stability in the pharmaceutical composition is at least about 50% of the recombinant AAV stability in the reference pharmaceutical composition.

46. The pharmaceutical composition of claim 45, wherein the recombinant AAV stability is determined by infectivity of the recombinant AAV.

47. The pharmaceutical composition of claim 45, wherein the recombinant AAV stability is determined by a level of free DNA released by the recombinant AAV.

48. The pharmaceutical composition of claim 47, wherein the pharmaceutical composition comprises at least about 50% more, about 25% more, about 15% more, about 10% more, about 5% more, about 4% more, about 3% more, about 2% more, about 1% more, about 0% more, about 1% less, about 2% less, about 5% less, about 7% less, about 10% less, about 2 times more, about 3 times more, about 2 times less, about 3 times less free DNA as compared to a level of free DNA in the reference pharmaceutical composition.

49. The pharmaceutical composition of claim 46, wherein the recombinant AAV in the pharmaceutical composition has an infectivity that is about 50% lower, about the same, or at least about 2%, 5%, 7%, 10%, 12%, 15%, 17%, 20%, 25%, 30% 35% 40%, 45%, 50%, 100%, 2 times, 3 times, 5 times, 10 times, 100 times, or 1000 times higher as compared to the infectivity of the recombinant AAV in the reference pharmaceutical composition.

50. The pharmaceutical composition of any one of claims 1-49, wherein the transgene is a transgene suitable to treat, or otherwise ameliorate, prevent or slow the progression of a disease of interest.

51. The pharmaceutical composition of any one of claims 1-50, wherein the human subject is diagnosed with nAMD (wet AMID), dry AMD, retinal vein occlusion (RVO), diabetic macular edema (DME), or diabetic retinopathy (DR), or Batten disease.

52. The pharmaceutical composition of any one of claims 1-50, wherein the human subject is diagnosed with glaucoma, non-infectious uveitis, or kallikrein-related disease.

53. The pharmaceutical composition of any one of claims 1-3, 4-9, and 10-52, wherein the AAV encodes Palmitoyl-Protein Thioesterase 1 (PPT1), Tripeptidyl-Peptidase 1 (TPP1), anti-TNF fusion protein, anti-kallikrein antibody or antigen-binding fragment, anti-TNF antibody or antigen-binding fragment, anti-C3 antibody or antigen-binding fragment, or anti-C5 antibody or antigen-binding fragment.

54. The pharmaceutical composition of any one of claims 4-53, wherein the amount of the recombinant AAV genome copies is based on a vector genome concentration.

55. The pharmaceutical composition of any one of claims 4-53, wherein the amount of the recombinant AAV genome copies is based on genome copies per administration.

56. The pharmaceutical composition of any one of claims 4-53, wherein the amount of the recombinant AAV genome copies is based on total genome copies administered to the human subject.

57. The pharmaceutical composition of claim 55, wherein the genome copies per administration is the genome copies of the recombinant AAV per suprachoroidal administration.

58. The pharmaceutical composition of claim 56, wherein the total genome copies administered is the total genome copies of the recombinant AAV administered suprachoroidally.

59. The pharmaceutical composition of claim 54, wherein the vector genome concentration (VGC) is of about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 2.5×10¹² GC/mL, about 3×10¹² GC/mL, about 5×10¹² GC/mL, about 6×10¹² GC/mL, about 1.5×10¹³ GC/mL, about 2×10¹³ GC/mL, or about 3×10¹³ GC/mL.

60. The pharmaceutical composition of any one of claims 56 and 58, wherein the total number of genome copies administered is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

61. The pharmaceutical composition of any one of claims 55 and 57, wherein the total number of genome copies per administration is about 6.0×10¹⁰ genome copies, about 1.6×10¹¹ genome copies, about 2.5×10¹¹ genome copies, about 3×10¹¹ genome copies, about 5.0×10¹¹ genome copies, about 6×10¹¹ genome copies, about 3×10¹² genome copies, about 1.0×10¹² genome copies, about 1.5×10¹² genome copies, about 2.5×10¹² genome copies, or about 3.0×10¹³ genome copies.

62. The pharmaceutical composition of any one of claims 2-61, wherein the pharmaceutical composition is administered once, twice, three times, four times, five times, six times, seven times, eight times, nine times, ten times, fifteen times, twenty times, twenty five times, or thirty times.

63. The pharmaceutical composition of any one of claims 4-62, wherein the reference pharmaceutical composition is administered once, twice, three times, four times, five times, six times, seven times, eight times, nine times, ten times, fifteen times, twenty times, twenty-five times, or thirty times.

64. The pharmaceutical composition of any one of claims 2-63, wherein the pharmaceutical composition is administered once in one day, twice in one day, three times in one day, four times in one day, five times in one day, six times in one day, or seven times in one day.

65. The pharmaceutical composition of any one of claims 4-63, wherein the reference pharmaceutical composition is administered once in one day, twice in one day, three times in one day, four times in one day, five times in one day, six times in one day, or seven times in one day.

66. The pharmaceutical composition of any one of claims 2-65, wherein the reference pharmaceutical composition comprises DPBS and sucrose.

67. A method of preparing a pharmaceutical composition comprising:
a. preparing a composition comprising phosphate-buffered saline, sucrose, and a recombinant adeno-associated virus (AAV) vector comprising an expression cassette encoding a transgene; and
b. admixing a solution comprising phosphate-buffered saline and sucrose to the composition,
wherein the pharmaceutical composition has lower ionic strength and/or a higher level of aggregated recombinant AAV than the composition.

68. A method of preparing a pharmaceutical composition comprising admixing a solution comprising phosphate-buffered saline and sucrose to a composition, wherein the composition comprises a recombinant adeno-associated virus (AAV) vector comprising an expression cassette encoding a transgene, and wherein the pharmaceutical composition has lower ionic strength and/or a higher level of aggregated recombinant AAV than the composition.

69. A kit comprising:
a. a composition comprising a recombinant adeno-associated virus (AAV) vector comprising an expression cassette encoding a transgene; and
b. a solution comprising phosphate-buffered saline and sucrose.

70. The kit of claim 69, wherein the kit further comprises instructions for admixing the composition with the solution.

71. The method of claim 68 or the kit of claim 69, wherein the composition comprises a phosphate-buffered saline and sucrose.

72. The method of any one of claims 67 and 71, or the kit of any one of claims 69 and 71, wherein the composition comprises 4% sucrose.

73. The method of any one of claims 67, 68, 71 and 72, or the kit of any one of claims 69-72, wherein the solution comprises 10% sucrose.

74. The method of any one of claims 67, 68, and 71-73, or the pharmaceutical composition of any one of claims 1-66, wherein the pharmaceutical composition has an ionic strength of about or of at most about 135 mM.

75. The method of any one of claims 67, 68, and 71-74, or the pharmaceutical composition of any one of claims 1-66, wherein the pharmaceutical composition has an ionic strength of about or of at most about 40 mM.

76. The method of any one of claims 67, 68, and 71-75, or the pharmaceutical composition of any one of claims 1-66, wherein the pharmaceutical composition has an ionic strength of about or of at most about 20 mM.

77. The method of any one of claims 67, 68, and 71-76, or the pharmaceutical composition of any one of claims 1-66, wherein the pharmaceutical composition has substantially the same tonicity or osmolality as the composition.

78. The method of any one of claims 67, 68, and 71-77, or the pharmaceutical composition of any one of claims 1-66, wherein at least some of the aggregated recombinant AAV in the pharmaceutical composition disaggregate after the pharmaceutical composition is administered to the suprachoroidal space of an eye of a human subject.

79. The method of any one of claims 67, 68, and 71-78, or the pharmaceutical composition of any one of claims 1-66, wherein aggregation of the recombinant AAV is reversed to unaggregated AAV or to monomers upon suprachoroidal administration of the pharmaceutical composition.

80. The method of any one of claims 67, 68, and 71-79, or the kit of any one of claims 69-72, wherein the composition comprises potassium chloride, potassium phosphate monobasic, sodium chloride, sodium phosphate dibasic anhydrous, sucrose, and optionally a surfactant.

81. The method of any one of claims 67, 68, and 71-80, or the kit of any one of claims 69-72 and 80, wherein the composition comprises modified Dulbecco's phosphate-buffered saline solution, and optionally a surfactant.

82. The method of any one of claims 67, 68, and 71-81, or the kit of any one of claims 69-72 and 80-81, wherein the composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and a surfactant.

83. The method of any one of claims 67, 68, and 71-82, or the kit of any one of claims 69-72 and 80-82, wherein the solution comprises a phosphate-buffered sodium chloride and sucrose.

84. The kit of claim 70, wherein the instructions comprise instructions on admixing the solution with the composition to obtain a pharmaceutical composition.

85. The method of any one of claims 67, 68, and 71-83, or the kit of claim 84, wherein the admixing the solution with the composition dilutes the composition by about or at least about 2-fold, 3-fold, 4-fold, 5-fold, 6-fold, 7-fold, 8-fold, 9-fold or 10-fold.

86. The method of any one of claims 67, 68, 71-83 and 85, or the kit of any one of claims 84-85, wherein the admixing the solution with the composition occurs on the same day that the pharmaceutical composition is administered to the suprachoroidal space of an eye of a human subject.

87. The method of any one of claims 67, 68, 71-83 and 85-86, or the kit of any one of claims 84-86, wherein the admixing the solution with the composition occurs within 24 hours of the pharmaceutical composition being administered to the suprachoroidal space of an eye of a human subject.

88. The method of any one of claims 67, 68, 71-83 and 85-87, or the kit of any one of claims 84-87, or the pharmaceutical composition of any one of claims 2-66 and 74-79, wherein the pharmaceutical composition is stored prior to administration to a human subject.

89. The method of any one of claims 67, 68, 71-83 and 85-88, or the kit of any one of claims 84-88, or the pharmaceutical composition of any one of claims 2-66, 74-79, and 88, wherein the pharmaceutical composition is stored at about room temperature, 20° C., 4° C., or −80° C.

90. The method of any one of claims 67, 68, 71-83 and 85-89, or the kit of any one of claims 84-89, or the pharmaceutical composition of any one of claims 2-66, 74-79, and 88-89, wherein the pharmaceutical composition comprises about 1.0×10¹² to about 3.0×10¹² genome copies of the recombinant AAV.

91. The method of any one of claims 67, 68, 71-83 and 85-90, or the kit of any one of claims 84-90, wherein the recombinant AAV comprises components from one or more adeno-associated virus serotypes selected from AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

92. The method of any one of claims 67, 68, 71-83 and 85-91, or the kit of any one of claims 84-91, or the pharmaceutical composition of any one of claims 2-66, 74-79, and 88-90, wherein the recombinant AAV comprises components from AAV8 and the pharmaceutical composition has an ionic strength between about 30 mM to about 60 mM.

93. The method of any one of claims 67, 68, 71-83 and 85-91, or the kit of any one of claims 84-91, or the pharmaceutical composition of any one of claims 2-66, 74-79, and 88-90, wherein the recombinant AAV comprises components from AAV9 and the pharmaceutical composition has an ionic strength between about 15 mM to about 30 mM.

94. The method of any one of claims 67, 68, 71-83 and 85-91, or the kit of any one of claims 84-91, or the pharmaceutical composition of any one of claims 1-66, 74-79, and 88-90, wherein the recombinant AAV comprises components from AAV2 and the pharmaceutical composition has an ionic strength between about 100 mM to about 200 mM.

95. The pharmaceutical composition of any one of claims 1-66, 74-79, and 88-94, wherein the pharmaceutical composition comprises modified Dulbecco's phosphate-buffered saline solution, and optionally a surfactant.

96. The pharmaceutical composition of any one of claims 1-66, 74-79, and 88-95, wherein the pharmaceutical composition comprises potassium chloride, potassium phosphate monobasic, sodium chloride, sodium phosphate dibasic anhydrous, sucrose, and optionally a surfactant.

97. The pharmaceutical composition of any one of claims 1-66, 74-79, and 88-96, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, and optionally a surfactant.

98. The pharmaceutical composition of any one of claims 1-66, 74-79, and 88-96, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4.

99. A pharmaceutical composition suitable for administration to the suprachoroidal space (SCS) of an eye of a human subject, wherein the pharmaceutical composition comprises a recombinant adeno-associated virus (AAV) vector comprising an expression cassette encoding a transgene, wherein the pharmaceutical composition comprises 0.2 mg/mL potassium chloride, 0.2 mg/mL potassium phosphate monobasic, 5.84 mg/mL sodium chloride, 1.15 mg/mL sodium phosphate dibasic anhydrous, 40.0 mg/mL (4% w/v) sucrose, 0.001% (0.01 mg/mL) poloxamer 188, pH 7.4.

100. The pharmaceutical composition of any one of claims 1-66, 74-79, and 88-99, wherein the pharmaceutical composition comprises a lower amount of AAV empty capsids as compared to a reference pharmaceutical composition.

101. The pharmaceutical composition of claim 100, wherein the amount of the AAV empty capsids in the pharmaceutical composition is lower by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% as compared to the amount of the AAV empty capsids in the reference pharmaceutical composition.

102. The pharmaceutical composition of any one of claims 1-66, 74-79, and 88-101, wherein the pharmaceutical composition comprises an ionic strength of about or at most about 5 mM, 10 mM, 15 mM, 20 mM, 25 mM, 30 mM, 35 mM, 40 mM, 45 mM, 50 mM, 55 mM, or 60 mM.

103. The pharmaceutical composition of any one of claims 100-102, wherein the reference pharmaceutical composition comprises an ionic strength of more than about 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, or more than about 200 mM.

104. A method of reducing or eliminating AAV empty capsids in a pharmaceutical composition, the method comprising:
a. introducing a solution comprising an ionic strength of at most about 60 mM to a formulation comprising a mixture of a recombinant adeno-associated virus (AAV) vector and AAV empty capsids; and
b. removing at least some of the AAV empty capsids from the formulation, wherein the formulation after step b is prepared into a pharmaceutical composition comprising the recombinant adeno-associated virus (AAV) vector, wherein the recombinant AAV vector comprises an expression cassette encoding a transgene, and wherein the pharmaceutical composition is suitable for administration to the suprachoroidal space (SCS) of an eye of a human subject.

105. A method of reducing or eliminating AAV empty particles in a population of AAV particles, wherein the population of AAV particles comprises empty AAV particles and AAV particles comprising an expression cassette encoding a transgene, and wherein the method comprises:
a. incubating the population of AAV particles in a solution at an ionic strength of at most about 60 mM, thereby creating aggregates of the AAV particles comprising the expression cassette encoding the transgene; and
b. removing at least a portion of the empty AAV particles from the population of AAV particles.

106. The method of claim 104, wherein the method further comprises introducing another solution comprising an ionic strength of at least about 80 mM to the formulation after step b.

107. The method of claim 105, wherein the method further comprises incubating the population of AAV particles after step b with another solution comprising an ionic strength of at least about 80 mM.

108. The method of claim 104 or 106, wherein the amount of the AAV empty capsids in the formulation is reduced by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% after step b. as compared to the amount of the AAV empty capsids in the formulation prior to step a.

109. The method of claim 105 or 107, wherein the amount of the empty AAV particles in the population of AAV particles is reduced by about or at least about 5%, 10%, 15%, 20%, 25%, 30%, 35%, 40%, 45%, 50%, 55%, 60%, 65%, 70%, 75%, 80%, 95%, 96%, 97%, 98%, 99%, or 100% after step b. as compared to the amount of the empty AAV particles in the population of AAV particles prior to step a.

110. The method of any one of claims 104-109, wherein the solution comprises an ionic strength of about 30 to about 50 mM.

111. The method of any one of claims 104-109, wherein the solution comprises an ionic strength of about 15 to 50 mM.

112. The method of any one of claims 104-109, wherein the solution comprises an ionic strength of at most about 50 mM.

113. The method of any one of claims 104-109, wherein the another solution comprises an ionic strength of about or at least about 150 mM.

114. A pharmaceutical composition produced by the method of any one of claims 104, 106, and 108-113.

115. A pharmaceutical composition comprising the population of AAV particles obtained after step b of the method of any one of claims 105 and 107-113.

116. The method or the kit of claim 81 or 82, wherein the solution comprises 10% Sucrose, 2.70 mM potassium chloride, 8.10 mM sodium phosphate dibasic anhydrous, 1.47 mM potassium phosphate monobasic, 292 mM sucrose, 0.001% (0.01 mg/mL) poloxamer 188, pH 7.4, and wherein the composition and solution are admixed at a composition to solution ratio of 1 to 9.


SEQUENCE LISTING (56 sequences)

SEQ ID NO: 1-4 - skipped (4 entries)

SEQ ID NO: 5 - protein, 26 aa, Synthetic construct
  source 1..26: note = VEGF-A signal peptide
MNFLLSWVHWSLALLLYLHHAKWSQA

SEQ ID NO: 6 - protein, 29 aa, Synthetic construct
  source 1..29: note = Fibulin-1 signal peptide
MERAAPSRRVPLPLLLLGGLALLAAGVDA

SEQ ID NO: 7 - protein, 19 aa, Synthetic construct
  source 1..19: note = Vitronectin signal peptide
MAPLRPLLILALLAWVALA

SEQ ID NO: 8 - protein, 18 aa, Synthetic construct
  source 1..18: note = Complement Factor H signal peptide
MRLLAKIICLMLWAICVA

SEQ ID NO: 9 - protein, 19 aa, Synthetic construct
  source 1..19: note = Opticin signal peptide
MRLLAFLSLLALVLQETGT

SEQ ID NO: 10-21 - skipped (12 entries)

SEQ ID NO: 22 - protein, 18 aa, Synthetic construct
  source 1..18: note = Albumin signal peptide
MKWVTFISLLFLFSSAYS

SEQ ID NO: 23 - protein, 18 aa, Synthetic construct
  source 1..18: note = Chymotrypsinogen signal peptide
MAFLWLLSCWALLGTTFG

SEQ ID NO: 24 - protein, 20 aa, Synthetic construct
  source 1..20: note = Interleukin-2 signal peptide
MYRMQLLSCIALILALVTNS

SEQ ID NO: 25 - protein, 15 aa, Synthetic construct
  source 1..15: note = Trypsinogen-2 signal peptide
MNLLLILTFVAAAVA

SEQ ID NO: 26 - protein, 19 aa, Synthetic construct
  source 1..19: note = F2A site
LLNFDLLKLAGDVESNPGP

SEQ ID NO: 27 - protein, 21 aa, Synthetic construct
  source 1..21: note = T2A site
GSGEGRGSLLTCGDVEENPGP

SEQ ID NO: 28 - protein, 22 aa, Synthetic construct
  source 1..22: note = P2A site
GSGATNFSLLKQAGDVEENPGP

SEQ ID NO: 29 - protein, 23 aa, Synthetic construct
  source 1..23: note = E2A site
GSGQCTNYALLKLAGDVESNPGP

SEQ ID NO: 30 - protein, 25 aa, Synthetic construct
  source 1..25: note = F2A site
GSGVKQTLNFDLLKLAGDVESNPGP

SEQ ID NO: 31 - protein, 4 aa, Synthetic construct
  source 1..4: note = Furin linker
RKRR

SEQ ID NO: 32 - protein, 4 aa, Synthetic construct
  source 1..4: note = Furin linker
RRRR

SEQ ID NO: 33 - protein, 4 aa, Synthetic construct
  source 1..4: note = Furin linker
RRKR

SEQ ID NO: 34 - protein, 4 aa, Synthetic construct
  source 1..4: note = Furin linker
RKKR

SEQ ID NO: 35-40 - skipped (6 entries)

SEQ ID NO: 41 - protein, 736 aa, Synthetic construct
  source 1..736: note = AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, Synthetic construct [sequence omitted]

SEQ ID NO: 43 - protein, 736 aa, Synthetic construct
  source 1..736: note = AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, Synthetic construct
  source 1..734: note = AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, Synthetic construct
  source 1..724: note = AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Synthetic construct
  source 1..736: note = AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, Synthetic construct
  source 1..737: note = AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, Synthetic construct
  source 1..738: note = AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, Synthetic construct
  source 1..736: note = hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, Synthetic construct
  source 1..736: note = hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, Synthetic construct
  source 1..736: note = AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - skipped

SEQ ID NO: 53 - protein, 306 aa, Synthetic construct
  source 1..306: note = Palmitoyl-protein thioesterase 1 (ppt1) Aah08426.1
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 54 - protein, 563 aa, Homo sapiens
  source 1..563: note = Tripeptidyl-peptidase 1 (tpp1) Np_000382.3
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 55 - protein, 20 aa, Homo sapiens
  source 1..20: note = Mutant interleukin-2 signal peptide
MYRMQLLLLIALSLALVTNS

SEQ ID NO: 56 - protein, 736 aa, Synthetic construct
  source 1..736: note = capsid protein VP1 for AAV3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL